(19) United States
(12) Patent Application Publication
(10) Pub. No.: US 2003/0157523 A1
(43) Pub. Date: Aug. 21, 2003

(54) Cell and tissue arrays and microarrays and methods of use

(75) Inventors: Gretchen Frantz, San Francisco, CA (US); Trent Harris Landon, San Francisco, CA (US); Franklin V. Peale, JR., San Carlos, CA (US); Thinh Quang Pham, San Bruno, CA (US); Jean Philippe F. Stephan, San Carlos, CA (US); Debra Y. Dunlap, Sunnyvale, CA (US); Kenneth J. Hillan, San Francisco, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/300,546

(22) Filed: Nov. 20, 2002

Related U.S. Application Data
(60) Provisional application No. 60/393,551, filed on Jul. 2, 2002.
(60) Provisional application No. 60/389,610, filed on Jun. 17, 2002.
(60) Provisional application No. 60/359,563, filed on Feb. 22, 2002.
(60) Provisional application No. 60/355,205, filed on Feb. 7, 2002.
(60) Provisional application No. 60/332,635, filed on Nov. 21, 2001.
(60) Provisional application No. 60/332,293, filed on Nov. 20, 2001.

(51) Int. Cl. C12Q 1/68; G01N 33/53; G01N 33/567; G01N 33/574; B28B 1/48; B29D 19/08; C12M 1/34

(52) U.S. Cl. 435/6; 435/7.2; 435/7.23; 435/287.2; 264/155

(57) ABSTRACT

The invention relates to biological arrays, biological microarrays, and methods of using the arrrays and microarrays to detect the amount and/or presence of a biological molecule in a biological sample. Biological arrays of the invention comprise a solidified, sectionable matrix comprising a plurality of wells disposed therein and one or more biological samples disposed within the plurality of wells, which biological arrays optionally comprise an internal standard preparation and/or an orientation marker. Sections or slices of the biological arrays are mounted on a planar substrate surface to form cellular microarrays of the invention. In alternative cellular microarrays of the invention, the matrix material is a temperature-sensitive material removable from the microarray leaving cellular biological material on the substrate surface.

This application is a non-provisional application filed under 37 CFR 1.53(b)(1), g claiming priority under 35 USC 119(e) to provisional application No. 60/393,551 filed Jul. 2, 2002, and to provisional application No. 60/389,610 filed Jun. 17, 2002, and to provisional application No. 60/359,563 filed Feb. 22, 2002, and to provisional application No. 60/355,205 filed on Feb. 7, 2002, and to provisional application No. 60/332,635 filed on Nov. 21, 2001, and to provisional application No. 60/332,293 filed on Nov. 20, 2001, the contents of which applications are incorporated herein by reference.

### FIELD OF THE INVENTION

The invention relates to an array of a plurality of biological samples, such as cell samples or tissue samples, in a matrix suitable for sectioning to produce multiple compositions useful for comparing biological properties of the biological samples in the array. The invention further relates to the array comprising an internal standard and uses of the multiple compositions for comparison of biological properties of the biological samples.

### BACKGROUND

The recent completion of the sequencing of the human genome and the genome of other organisms has provided a tremendous amount of information to the scientific community. Based on this technology, biotechnology and pharmaceutical industries have developed strategies to identify candidate molecules for potential therapeutic applications. The next objective is to harness this vast wealth of genetic data in the prediction, diagnosis, and treatment of diseases. However, in order to make sense of this information, high efficiency analytical technologies are required.

Known approaches for identifying genes or gene products unique to a particular type of cell or tissue are generally limited, targeting only one or a few specific gene sequences, and analyzing one cell type or tissue type at a time. More recently, high throughput methods have been devised to identify genes or gene products in multiple different cell or tissue samples. One such high throughput method consists of a making an array of cell or tissue samples. To make an array, cell and/or tissue samples are typically inserted into tubes within a three-dimensional solid array recipient block made of a matrix material, such as paraffin or gelatin. The paraffin array recipient block is then cut into one or more thin slices, each slice containing the same array of samples. The array slice is applied to a microscope slide and the matrix is removed, leaving an array of cell and/or tissue samples in an array of spots or transverse sections of cell sample or tissue sample. The array slides, also called tissue microarrays, are useful for a variety of analytical procedures or molecular analyses, such as in-situ hybridization and immunochemistry procedures. Although known arrays allow for multiple molecular analyses of multiple different cell or tissue samples in an efficient manner, these arrays have many disadvantages, particularly in the method of production.

One disadvantage of the known arrays is that during preparation some samples, such as cell suspensions, must be retained in a barrier material, such as glass or plastic tubes, within the solid matrix of the array. The presence of the tubes hampers slicing of the array. Further, when the array is sliced, the tubes tend to break and disturb the samples. Accordingly, there is substantial need for a method and apparatus for making an array that can be easily sliced and that does not require tubes to retain the samples.

Another disadvantage of the known arrays is that cell or tissue samples require fixation and extensive handling for preservation prior to being inserted into the tubes of the array. The fixatives can damage the cell or tissue samples, which in turn can affect the integrity of the results of any molecular analysis or analytical procedure performed on the array slides. The extensive handling of the samples also can cause damage to the samples and is very time consuming. Accordingly, there remains a significant need for a method and apparatus for the manufacture of an array that does not require chemical treatment or extensive handling of samples prior to insertion into an array.

In addition, it would be advantageous to improve the methods for qualitative and quantitative analysis of data generated from an array. In general, analysis of these results typically requires a person to inspect each spot on an array slide with a microscope, and to record the results qualitatively (e.g. −, +, or +/−) for each of the spots on a single slide. This procedure is time consuming, error prone, and provides very limited quantitative information, for example, being limited to the levels of signal intensity visible to the human eye. Further, where quantitation of expression in tissue sections is desired, existing methods require that standards are analyzed separately thereby limiting their usefulness (Ermert, L. et al., Am. J. Path. 158:407-417 (2001). Accordingly, there remains a substantial need for an efficient method for analyzing the results of molecular analyses of samples on an array slide. Additionally, there is a substantial need for providing an accurate and efficient means for quantitating the results of molecular analyses of samples on an array slide.

### SUMMARY OF THE INVENTION

Against this backdrop the present invention has been developed to solve the above and other problems. The present invention generally comprises a frozen biological array and method and apparatus for making a frozen array that eliminate the need for a barrier material between an array matrix and a biological sample and further eliminate the need to chemically process a sample before using it in the array. Additionally, the present invention comprises a biological array, either frozen or not, and a method of making an array containing an internal standard preparation that aids in the analysis of biological samples contained within arrays.

In one embodiment, a frozen biological array comprises a frozen matrix formed of a temperature-sensitive material having a plurality of wells disposed therein and one or more biological samples disposed within the plurality of wells within the frozen sectionable matrix. In an embodiment, a frozen array recipient block capable of receiving one or more frozen biological samples to create a frozen array may be made as follows. An arrayer having a plurality of pins is engaged with an embedding mold and a fluid temperature-sensitive matrix such that the matrix and the pins are contained within the embedding mold. In alternative embodiments, the matrix is poured into the mold, and the matrix is engaged with the mold prior to engaging the arrayer pins with the matrix. For example, in an embodiment, the arrayer pins are inserted into the fluid matrix within the embedding mold. Alternatively, the arrayer pins may be engaged with the mold prior to engaging the fluid matrix with the mold and the arrayer pins. While the temperature-sensitive matrix is engaged with the pins and the mold, the matrix is frozen causing the fluid matrix to solidify around the pins of the arrayer. When the pins are removed from the matrix and embedding mold, a plurality of wells are disposed within the frozen temperature-sensitive matrix.

It is disclosed herein that coating the pins of the arrayer with a lubricating material eases removal of the pins from the frozen temperature-sensitive matrix. As a result, an embodiment of the invention involves a method of making the frozen biological array by coating the pins with a lubricating material such as, but not limited to, glycerol, fatty acids, oil, grease, fat, or soap, prior to contacting the temperature-sensitive matrix and freezing.

In another embodiment, the invention involves a frozen array comprising a plurality of wells lined with a lubricating material. In still another embodiment, the invention involves a frozen biological array comprising a plurality of wells lined with a lubricating material and containing a biological sample such as, but not limited to, a cell suspension, cell pellet, cell lysate, a tissue, where the lubricating material forms a thin film lining the well between the frozen temperature-sensitive matrix and the cell or tissue sample.

In another aspect, the invention involves an apparatus for making an array recipient block comprising an arrayer having a body and a plurality of pins protruding from the body, an embedding mold for containing a temperature-sensitive matrix, and the temperature-sensitive matrix material. According to the invention, the arrayer body comprises more than 5 pins/cm², alternatively more than 7 pins /cm², or alternatively more than 13 pins /cm². Also, according to the invention, a cross-section of a biological array of the invention comprises more than 5 wells/cm², alternatively more than 7 wells/cm², alternatively more than 11 wells /cm², or alternatively more than 13 wells /cm². In an embodiment, the wells are evenly spaced within the matrix. In another embodiment, one or more of the wells has a circular cross section. In still another embodiment, one or more of the wells have an internal diameter in a range of about 0.4 mm to about 1.2 mm, about 0.4 mm to about 0.7 mm, or about 0.8 mm to about 1.2 mm.

In another aspect, the invention involves a cellular microarray made by inserting one or more biological samples into the plurality of wells within the frozen array recipient block to create a frozen biological array, slicing the frozen array into one or more sections, mounting the sections on a planar substrate surface, such as microscope slide, and removing the matrix material from the platform to form a cellular microarray. In an embodiment, the biological sample is a cell suspension and the sections of frozen array comprise transverse sections (or spots) of cell suspension sample. In another embodiment, the biological sample is a tissue sample and the sections of frozen array comprise transverse sections (or spots) of tissue sample. In another embodiment, the microarray invention involves a cellular microarray in which the transverse sections (or spots) of the biological samples of a biological array are surrounded by an area of lubricating material between the biological sample transverse section and the OCT on the planar surface. According to the invention, the slices of array or microarray comprise more than 5 transverse sections/cm², alternatively more than 7 transverse sections /cm², more than 11 transverse sections/cm², or more than 13 transverse sections /cm². In a further embodiment, the wells of the biological array are lined with a lubricating material following removal of the pins easing slicing of the biological sample in the wells such that a microarray according to this embodiment comprises biological sample transverse sections having cleaner edges than in the absence of such lubricating material lining the wells.

In yet another aspect, the invention involves a biological array comprising a matrix having a plurality of wells disposed therein, samples contained in some of the wells, and one or more internal standard preparations contained in some of the wells. The internal standard preparation comprises a standard molecule, such as biological molecule, admixed in an embedding material. The embedding material differs from the matrix in at least one physical or chemical property such that the internal standard preparation will retain the standard molecule in the array and on a microarray substrate during processing and any procedures performed on the array or the microarray. The internal standard preparation aids in analyzing results of procedures performed on the array or microarray in a number of ways, including by acting as a positive or negative control and assisting in detecting and quantitating a biological molecule in the samples in the array or microarray.

In one embodiment, the internal standard preparation comprises a polynucleotide, such as an RNA or DNA molecule, admixed in agarose with or without BSA to form an internal standard preparation that aids analyzing the results of an in-situ hybridization procedure performed on an array containing these internal standard preparations. In an embodiment the RNA or DNA molecule is single stranded. In another embodiment, the polynucleotide hybridizes to a probe used for detecting the presence of the polynucleotide in the standard. In another embodiment, the biological molecule comprises a polypeptide admixed in agarose with or without BSA to form an internal standard preparation that aids in analyzing a immunohistochemistry procedure performed on an array containing the protein internal standard preparation. In yet another embodiment, the internal standard preparation may contain two or more biological molecules, including two or more polynucleotides, two or more polypeptides, or any combination thereof.

In another embodiment, a standard orientation molecule, such as a dye or a non-specific binder of probes, may be admixed in an embedding material to act as an orientation marker in an array or microarray.

In another aspect, the invention involves a microarray that is made by inserting one or more internal standard preparations into the plurality of wells within an array recipient block, either frozen or not, to create a biological array, slicing the array into one or more sections, mounting the sections on a planar substrate surface, such as microscope slide, and, if the array is a frozen array, removing the matrix material from the surface, to form a cellular microarray. In an embodiment, the biological sample is a cell suspension and the sections of array comprise transverse sections (or spots) of cell suspension sample and internal standard preparations. In another embodiment, the biological sample is a tissue sample and the sections of array comprise transverse sections (or spots) of tissue sample and internal standard preparation.

In another aspect, the invention involves a cellular microarray that is made by preparing a matrix having a plurality of wells disposed therein, making an internal standard preparation and inserting the internal standard preparation into one or more of the plurality of wells in the matrix, and inserting a biological sample into one or more of the plurality of wells in the matrix to form a cellular array. The cellular array is then sliced into one or more array slices, mounted on a planar substrate, and the matrix is removed from the substrate. The cellular microarray comprises a substrate having a planar surface; one or more cellular biological samples on the surface; and one or more internal standard preparations on the surface, the internal standard preparation comprising a standard molecule admixed in an embedding material. In another embodiment, a cellular microarray comprises a substrate comprising a planar surface and one or more cellular biological samples on the surface, wherein the microarray lacks array matrix material.

In another aspect, the invention involves a method for detecting a biological molecule in an array or microarray comprises the following. A known quantity of a biological molecule is mixed with an embedding material so as to provide an internal standard preparation. The internal standard preparation is inserted into one or more of the wells in an array recipient block. One or more samples are inserted into the wells in the array recipient block, thereby forming an array. An analytical procedure is performed on the array and a result of the analytical procedure on the internal standard preparation is correlated to a result of the analytical procedure on the sample to determine detection of the biological molecule in the sample. According to the invention, embodiments of the method of detecting include without limitation in-situ hybridization, immunohistochemistry, binding of a receptor (or ligand-binding fragment of a receptor, such as an ECD) to a ligand wherein such binding is detected by labeling the receptor, the ligand, or a third molecule (such as an antibody), which third molecule specifically binds the receptor-ligand complex. According to the method of the invention, detecting is alternatively accomplished by detecting the specific association (such as by binding or by hybridization) of a detectably labeled molecule with a biological molecule in of interest. According to the invention, detection is performed by an instrument, such as, but not limited to, a phosphoroimager, a fluorescence detection device, a photographic film, a visible light detector, a detector of chemiluminesence, and a CCD camera.

In another aspect, the invention involves detecting a disease state in a biological sample of a patient relative to a control, non-diseased state. Embodiments of the invention include, but are not limited to, detection, using the microarrays and microarray methods of the invention, of a biological molecule in an array, wherein the amount of the biological molecule in a sample differs from the amount in a normal sample. In one embodiment, the biological molecule is at least 1.5 fold overexpressed in the sample array cells or tissue relative to a control tissue or cells. According to one embodiment, the invention involves detection of cancer in a breast tissue sample by contacting a polynucleotide comprising at least 20 contiguous nucleotides of the Her2 gene (or its complement) with a sample in a tissue microarray or frozen cell microarray of the invention and detecting overexpression of the Her2 gene relative to a control sample. According to another embodiment, the invention involves detection of cancer in a breast tissue sample by contacting a HER2-binding agent, such as an antibody or HER2-binding fragment thereof, with a biological sample in a tissue microarray or frozen cell microarray of the invention and detecting overexpression of HER2 protein relative to a control sample. In yet another embodiment, the invention involves identifying a patient disposed to respond favorably to an ErbB antagonist for treating cancer, which method comprises detecting erbB gene amplification in tumor cells in a tissue sample from the patient by detecting gene amplification or protein overexpression using a tissue microarray or cell microarray of the invention as disclosed herein above. Disposition of the patient for favorable response to an ErbB antagonist is disclosed in pending application Ser. No. 09/863,101, filed May 18, 2001, hereby incorporated by reference in its entirety.

In still another embodiment, the invention involves, detection of overexpression of VEGF, including detection of cancer, in a biological sample by contacting a polynucleotide comprising at least 20 contiguous nucleotides of the VEGF gene (or its complement) with a sample in a tissue microarray or frozen cell microarray of the invention and detecting overexpression of the VEGF gene relative to a control sample. According to another embodiment, the invention involves detection of cancer in a sample by contacting a VEGF-binding agent, such as an antibody or VEGF-binding fragment thereof, with a biological sample in a tissue microarray or frozen cell microarray of the invention and detecting overexpression of VEGF protein relative to a control sample.

These and various other features as well as advantages which characterize the present invention will be apparent from a reading of the following detailed description, including the examples, and a review of the associated drawings.

### BRIEF DESCRIPTION OF THE DRAWINGS

FIG. 1 shows an exploded perspective view of an arraying apparatus prior to insertion of an arrayer into a mold in accordance with one embodiment of the invention.

FIG. 2 is a sectional view through lines 2-2 of FIG. 1 after the arrayer has been inserted into the matrix in the mold, the matrix has been frozen, and the arrayer has been removed to form an array recipient block.

FIG. 3 is a perspective view of the array recipient block of FIG. 2 in accordance with one embodiment of the invention.

FIG. 4 is a top view of a cell or tissue microarray comprising a slide containing two array slices in accordance with one embodiment of the invention.

FIG. 5 is an exploded section view of an arraying apparatus after the arrayer has been inserted into the matrix in the mold, the matrix has been frozen, and the arrayer has been removed in accordance with an alternative embodiment of the invention.

FIG. 6 is an exploded section view of an arraying apparatus after the arrayer has been inserted, the matrix has been frozen, and the arrayer has been removed in accordance with yet another alternative embodiment of the invention.

FIG. 7 is a top view of a cell or tissue microarray containing three spots 504 (or transverse sections) of internal standard preparation and twelve spots 506 (or transverse sections) of biological sample in accordance with one embodiment of the invention.

FIGS. 8A-8B are bar graphs of the relative amount of HER2 gene amplification (FIG. 8A) and HER2 protein levels (FIG. 8B (ELISA) and in HER2-expression cell lines on a microarray (FIGS. 8C to 8F (quantitative immunofluorescence according to the invention)).

FIGS. 9A-9D are photographs of tissue microarrays on which HER2-expressing cells were used as controls and HER2 ECD protein embedded in agarose was used as standards. HER2 was immunostained in ninety nine cases of paraffin-embedded grade 3 ductal breast cancers, HER2-expressing cell lines, and HER2 ECD standards. FIG. 9A depicts immunofluorescence detection of goat anti-human HER2 ECD polyclonal antibody binding to HER2 using Alexa Fluor 633. FIG. 9B depicts immunofluorescence detection of rabbit rabbit anti-human c-erbB2 (HER2/neu) polyclonal antibody binding to HER2 using Alexa 633. FIG. 9C depicts mmunohistochemical detection of goat anti-human HER2 ECD polyclonal antibody binding to HER2 using immunoperoxidase. FIG. 9D depicts immunohistochemical detection of rabbit rabbit anti-human c-erbB2 (HER2/neu) polyclonal antibody binding to HER2 using immunoperoxidase.

FIG. 10 shows a tissue microarray conataining orientation markers positioned with the array. Non-specific binding of a labeled polynucleotide probe to the markers (arrows) is shown in a phosphorimage of the array. The orientation markers comprise microgranular cellulose and agarose as described in Example 14.

FIGS. 11A-11D are images of tissue microarrays containing cellulose/agarose internal standard preparations. FIG. 11A shows the autofluorescence phosphorimager signal results of the hybridization with an anti-sense Her2/ErbB2 probe on an array containing the cellulose/agarose internal standard preparations described in Example 14. FIG. 11B shows the autofluorescence phosphorimager signal results of the hybridization with a sense Her2/ErbB2 probe on an array containing the cellulose/agarose internal standard preparations described in Example 14. FIG. 11C shows the ISH phosphorimager signal results of the hybridization with an anti-sense Her2/ErbB2 probe on an array containing the cellulose/agarose internal standard preparations described in Example 14. FIG. 11D shows the ISH phosphorimager signal results of the hybridization with a sense Her2/ErbB2 probe on an array containing the cellulose/agarose internal standard preparations described in Example 14.

FIG. 12 shows a photograph of a top view of the array containing an assymetrical pattern of dye/agarose internal standard preparations as described in Example 15.

### DESCRIPTION OF THE EMBODIMENTS

Definitions

As used herein the term “adult organism” shall mean an organism that has reached full growth and development. In contrast, a “pre-adult stage of development” as applied to an organism shall mean an organism that has not yet reached full growth and development.

As used herein the term “amino acid” refers to either natural and/or unnatural or synthetic amino acids, including glycine and both the D or L optical isomers, and amino acid analogs and peptidomimetics.

As used herein, the terms “antibodies” and ‘immunoglobulins” refer to glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Native antibodies and immunoglobulins are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al. (1985) J. Mol. Biol. 186, 651-663; Novotny and Haber (1985) Proc. Natl. Acad. Sci. USA 82:4592-4596).

The light chains of antibodies (immunoglobulins) from any vertebrate species can be assigned to one of two clearly distinct types, called kappa and lambda (8), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g. IgG-1, IgG-2, IgG-3, and IgG-4; IgA-1 and IgA-2. The heavy chain constant domains that correspond to the different classes of immunoglobulins are called ″, delta, epsilon, (, and μ, respectively. The subunit structures and three-dimensional configurations of different classes of immunoglobulins are well known.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, monoclonal antibodies to be used in accordance with the present invention may be made by the hybridoma method first described by Kohler and Milstein (1975) Nature 256:495, or may be made by recombinant DNA methods (see, e.g. U.S. Pat. No. 4,816,567 (Cabilly et al.) and Mage and Lamoyi (1987) in Monoclonal Antibody Production Techniques and Applications, pp. 79-97, Marcel Dekker, Inc., New York). The monoclonal antibodies may also be isolated from phage libraries generated using the techniques described in McCafferty et al. (1990) Nature 348:552-554, for example.

“Humanized” forms of non-human (e.g. murine) antibodies are specific chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. For the most part, humanized antibodies are human immunoglobulins (recipient antibody) in which residues from the complementarity determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues which are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin. For further details see: Jones et al. (1986) Nature 321:522-525; Reichmann et al. (1988) Nature 332:323-329; EP-B-239 400 published Sep. 30, 1987; Presta (1992) Curr. Op. Struct. Biol. 2:593-596; and EP-B-451 216 published Jan. 24, 1996), which references are herein incorporated by reference in their entirety. The humanized antibody includes a Primatized™ antibody wherein the antigen-binding region of the antibody is derived from an antibody produced by immunizing macaque monkeys with the antigen of interest.

An “antigen” as used herein means a substance that is recognized and bound specifically by an antibody, a fragment thereof, or by a T cell antigen receptor. Antigens can include peptides, proteins, glycoproteins, polysaccharides, lipids, portions thereof, and combinations thereof. Antigens can be found in nature or can be synthetic. Antigens may be present on the surface of or located within a cell.

The term “anti-sense” is used to refer to a particular sequence orientation of a nucleic acid. When used to refer to DNA sequence orientation, “anti-sense strand” shall mean a strand of DNA, such as in a DNA duplex, that serves as a template for messenger RNA (mRNA) transcription. A “sense strand” of DNA shall mean a strand of DNA complementary to an anti-sense strand of DNA, which sense strand does not function as a template for mRNA synthesis. The sense DNA strand and the mRNA, which was synthesized from the template anti-sense DNA, have the same nucleotide sequence except that uracil (U) of mRNA substitutes for thymidine (T) of DNA. As a result, the sequence orientation of naturally occurring mRNA is frequently said to be in the sense orientation because it is complementary to the anti-sense DNA from which it was transcribed and because its sequence is similar to that of the sense DNA strand. In general, anti-sense RNA occurs only rarely in nature, but is a typical reagent used in in-situ hybridization procedures.

The term “arrayer” shall mean a tool, apparatus, or instrument designed to produce or create one or more wells in an array matrix. A non-limiting example of an arrayer useful in preparation of a tissue array and tissue microarray is described by Leighton, S. B., in U.S. Pat. No. 6,103,518, herein incorporated by reference in its entirety with respect to arrayer devices and their uses.

The term “biological array” as used herein, and as further described herein, refers to a sectionable block, such as a paraffin or frozen block, that typically contains between 25 to more than one thousand individual biological samples, such as tissue, cell suspensions, or cell pellets, as a pattern (such as an array (rows and columns)) of cores of biological samples, each core having been embedded at a specific grid coordinate location in the sectionable block, where each grid coordinate is sufficiently separate from every other grid coordinate such that material from each biological sample is separate and such that, when sectioned and mounted on a planar substrate, material from each biological sample is separate and separately detectable from material in every other biological sample. According to the invention, the biological sample in each well is contained within the well by the solidified matrix material that forms the walls of the well, and not by a tube or other non-matrix material forming a wall of the well. The term “biological array” includes, but is not limited to, “tissue arrays,” “cell arrays,” “frozen cell arrays,” or “frozen tissue arrays” as defined herein.

The term “biological molecule” as used herein refers to any organic molecule that is an essential is part of or derived from a molecule found in a living organism, including, but not limited to, polynucleotides, different orientations (sense or anti-sense) or splice variants of polynucleotides, polypeptides, and/or different isoforms of proteins (full-length or partial sequences), as well as non-polymeric molecules such as hormones, cytokines, metabolites, metabolic precursors, drugs or other chemicals used to treat a biological sample under investigation, and synthetic forms of such molecules.

The term “biological sample” or “cellular biological sample” as used herein refers to a sample of a cell population, such as a population of whole cells in a suspension or cell pellet or a cell lysate from a population of cells, and further refers to a tissue sample comprising whole and/or broken or lysed cells. The cell or tissue may be from any prokaryotic or eukaryotic organism including, but not limited to, bacteria, yeast, insect, bird, reptile, and any mammal including human. Where the cell or tissue is mammalian, the cell or tissue is any cell or tissue including, but not limited to blood, muscle, nerve, brain, breast, prostate, heart, lung, liver, pancreas, spleen, thymus, esophagus, stomach, intestine, kidney, testis, ovary, uterus, hair follicle, skin, bone, bladder, and spinal cord.

The term “cell pellet” as used herein refers to a sample in which cells are packed together into a mass, such as by centrifugation, for the purpose of concentrating a cell suspension, removing supernatant, and/or preparing a histological sample, such as a frozen cell array or microarray.

A “cell suspension” is a sample in which cells are more or less evenly dispersed in a liquid phase.

A “control” is an alternative subject or sample used in an analytical procedure for comparison purposes. A control can be “positive” or “negative”. For example, where the purpose of an analytical procedure is to detect a differentially expressed transcript or polypeptide in cells or tissue affected by a disease of concern, it is generally helpful to include a positive control, such as a subject or a sample from a subject exhibiting the desired expression and/or clinical syndrome characteristic of the desired expression, and a negative control, such as a subject or a sample from a subject lacking the desired expression and/or clinical syndrome of that desired expression. A control may or may not include a standard molecule as defined herein for the purpose of detecting and/or quantitating the amount of a target molecule in a sample.

A “dectectably labeled compound” shall mean a compound that is capable of attaching to or binding a biological molecule and has a label that is capable of being detected by any analytical procedure performed on the biological molecule. The term “label” refers to a moiety that, when attached to a compound (such as a nucleotide, polynucleotide, polypeptide, antibody or antigen binding fragment thereof, receptor or ligand binding fragment thereof, a receptor ECD, antigen, or receptor ligand, biotin, avidin, or streptavidin), renders such compound detectable using known detection means. Exemplary nonlimiting labels include fluorophores, chromophores, radioisotopes, spin-labels, enzyme labels, chemiluminescent labels, luminescent labels and the like, which allow direct detection or a labeled compound by a suitable detector, or a ligand, such as an antigen, or biotin, which can bind specifically with high affinity to a detectable anti-ligand, such as a labeled antibody or avidin. Where the labeled compound is a labeled antibody, the label may be conjugated directly or indirectly to the antibody so as to generate a “labeled” antibody. The label may be detectable by itself (e.g. radioisotope isotope or fluorescent label) or, in the case of an enzymatic label, may catalyze chemical alteration of a substrate compound or composition which is detectable.

“Differentially expressed,” as applied to a nucleotide sequence or a polypeptide sequence in a sample, refers to over-expression or under-expression of the sequence when compared to its expression as detected in a control. Underexpression also encompasses absence of expression of a particular sequence as evidenced by the absence of detectable expression in a sample when compared to a control.

“Differential expression” or “differential representation” refers to alterations in the abundance or the expression pattern of a gene product. An alteration in “expression pattern” may be indicated by a change in tissue distribution, or a change in hybridization pattern reviewed on an array of the invention.

The term “diseased cell” or “diseased tissue” refers to a state of a cell or tissue in which the cell or tissue that is biologically negatively compromised relative to a normal cell or tissue. Example of disease states include, but are not limited to, cancer, inflammation, apoptosis, and abnormal gene expression. The diseased cell or diseased tissue may be from any prokaryotic or eukaryotic organism including, but not limited to, bacteria, yeast, insect, bird, reptile, and any mammal including human. Where the cell or tissue is mammalian, the cell or tissue is any cell or tissue including, but not limited to blood, muscle, nerve, brain, breast, heart, lung, liver, pancreas, spleen, thymus, esophagus, stomach, intestine, kidney, testis, ovary, uterus, hair follicle, skin, bone, bladder, and spinal cord.

A “donor block” refers to any solid or semi-solid substance from which a sample may be taken for insertion into an array, including for example, a block of frozen tissue or paraffin-embedded tissue or a block of an internal standard preparation as described herein. The sample or core may be taken from the donor block by any means, including, but not limited to, using a typical arraying instrument, such as a Beecher arraying instrument.

As used herein, “expression” refers to the process by which a polynucleotide is transcribed into mRNA and/or the process by which the transcribed mRNA (also referred to as “transcript”) is subsequently translated into peptides, polypeptides, or proteins.

The term “embedding material” as used herein refers to any material in which a standard molecule, as defined herein, can be homogeneously suspended in a liquefied form of the embedding material which when solidified, before or during insertion into a well of a biological array, forms a solid internal standard preparation that is homogeneous with respect to distribution of the standard molecule in the preparation. In an embodiment of the invention, the solidification of the liquefied embedding material occurs by cooling. In another embodiment of the invention, the solidification of the embedding material is not catalyzed by an enzymatic reaction to cause gelling, The solidified embedding material of the internal standard preparation thereafter retains the standard molecule in the array or on a microarray substrate throughout processing and analytical procedures performed on the array or array slide, including procedures designed to remove an array matrix. Embedding material can include, but is not limited to, agarose (such as 1-4% agarose), bovine serum albumin (BSA, such as 1-20% BSA), a mixture of agarose and BSA, and the like.

The term “fluid” shall mean a state of matter that is able to flow or move freely, such as a liquid or soft gel, but not a gas.

The terms “frozen cell array” and “frozen tissue array’ as used herein, and as further described herein, refer to a sectionable block of frozen matrix material in which wells are filled with frozen tissue or concentrated cell suspensions, and where the wells are configured in a pattern, such as rows and columns, to form an array in the sectionable block.

A “gene” refers to a polynucleotide containing at least one open reading frame that is capable of encoding a particular protein after being transcribed and translated.

The term “hybridize” as applied to a polynucleotide refers to the ability of the polynucleotide to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. The hybridization reaction may constitute a step in a more extensive process, such as the initiation of a PCR reaction, or the enzymatic cleavage of a polynucleotide by a ribozyme. When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “in-situ hybridization” shall mean the use of a probe to detect the presence of the complementary DNA or RNA sequence in cloned bacterial or cultured eukaryotic cells, such as in thin sections of tissue or standard material incorporated into embedding material. “In-situ hybridization” is a well-established technique that allows specific polynucleotide sequences to be detected in morphologically preserved chromosomes, cells, tissue sections, or whole tissue fragments. In combination with immunocytochemistry, in-situ hybridization can relate microscopic topological information to gene activity at the DNA, mRNA, and protein levels.

The term “internal standard preparation” shall mean a mixture of a standard molecule, as defined herein, with an embedding material, as defined herein, that is used in an array to aid in an analysis of the array. For example, the internal standard preparation may be used to detect a biological molecule in a sample in a biological array, such as a positive or negative control (as defined herein) in a biological array, or for quantitation of a biological or target molecule in an array. Where quantitation is intended, the internal standard is present in the preparation at a known quantity or a determinable quantity.

“In-vitro” studies are those carried out outside of living organisms. “In-vivo” studies are those carried out within living organisms.

A “ligand” refers to a molecule capable of being bound by the ligand-binding domain of a receptor. The molecule may be chemically synthesized or may occur in nature.

“Luminescence” is the term commonly used to refer to the emission of light from a substance for any reason other than a rise in its temperature. In general, atoms or molecules emit photons of electromagnetic energy (e.g., light) when they move from an “excited state” to a lower energy state (usually the ground state); this process is often referred to as “radiative decay”. There are many causes of excitation. If the exciting cause is a photon, the luminescence process is referred to as “photoluminescence”. If the exciting cause is an electron, the luminescence process is referred to as “electroluminescence”. More specifically, electroluminescence results from the direct injection and removal of electrons to form an electron-hole pair, and subsequent recombination of the electron-hole pair to emit a photon. Luminescence that results from a chemical reaction is usually referred to as “chemiluminescence”. Luminescence produced by a living organism is usually referred to as “bioluminescence”. If photoluminescence is the result of a spin-allowed transition (e.g., a single-singlet transition, triplet-triplet transition), the photoluminescence process is usually referred to as “fluorescence”. Typically, fluorescence emissions do not persist after the excitation source is removed as a result of short-lived excited states, which may rapidly relax through such spin-allowed transitions. If photoluminescence is the result of a spin-forbidden transition (e.g., a triplet-singlet transition), the photoluminescence process is usually referred to as “phosphorescence”. Typically, phosphorescence emissions persist long after the exciting cause is removed as a result of long-lived excited states which may relax only through such spin-forbidden transitions. A “luminescent label” may have any one of the above-described properties.

The term “matrix” shall mean the material used to form the block used in biological arrays. The “matrix material” may be any material capable of forming a solid state with wells disposed therein, however, the “matrix material” must differ from the embedding material (as defined herein) by at least one physical or chemical property. After the array is sliced and the array slice is placed on a planar surface, such as a platform or slide, the matrix material is removed to form a microarray.

The terms “microarray,” “array slide,” “biological microarray,” and “cellular microarray” are used interchangeably to refer to thin sections of a biological array (defined herein) mounted on a planar platform or substrate, such as a glass microscope slide or other planar rigid surface including, but not limited to, glass, plastic, metal, silicon wafer, and the like, which surface is compatible with the selected method of screening. The thin sections (from 0.5-30 μm, alternatively from 5-15 μm, alternatively from 6-12 μm) are mounted on a planar substrate such that the separate and separated biological samples form a pattern of separated samples (such as a pattern of rows and columns as in a grid or an array) on the platform. According to the invention, the samples are not separated by sections of tubes or other rigid devices or barrier materials used to contain a cell or tissue sample in the wells of a biological array. Microarrays allow the examination of a large series of specimens while maximizing efficient utilization of technician time, reagents, and valuable tissue resources. Microarrays can be used for rapid, large-scale screening of tissue expression patterns of potential therapeutic targets and studies of molecular markers associated with prognosis and response to therapy.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “normal” sample refers to tissue or cells that are not diseased as defined herein. The term “normal cell” or “normal tissue” as used herein refers to a state of a cell or tissue in which the cell or tissue that is apparently free of an adverse biological condition when compared to a diseased cell or tissue having that adverse biological condition. The normal cell or normal tissue may be from any prokaryotic or eukaryotic organism including, but not limited to, bacteria, yeast, insect, bird, reptile, and any mammal including human. Where the cell or tissue is mammalian, the cell or tissue is any cell or tissue including, but not limited to blood, muscle, nerve, brain, breast, heart, lung, liver, pancreas, spleen, thymus, esophagus, stomach, intestine, kidney, testis, ovary, uterus, hair follicle, skin, bone, bladder, and spinal cord.

The terms “nucleic acid sequence” and “polynucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs: If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “optimal cutting temperature medium,” “OCT medium,” and “OCT” are used interchangeably herein and refer to a chemical formulation that, when solid (such as by freezing), can be cut and handled in thin sections typically of approximately 6 microns or micrometers to approximately 12 microns or micrometers, which sections are subsequently applied to a planar surface to generate a frozen tissue microarray or, as disclosed here, a frozen cell microarray. OCT generally comprises resin-polyvinyl alcohol, benzalkonium chloride to act as an antifungal agent, and polyethylene glycol to lower the freezing temperature. OCT mediums, such as those manufactured by Lab-Tek Instruments Co., Westmont Ill., come in three types for three ranges of temperature, −10° C. to −20° C., −20° C. to −35° C., and −35° C. −50° C.

The term “oligonucleotide” as used herein refers to a single stranded DNA or RNA molecule, typically prepared by synthetic means. Those oligonucleotides employed in the present invention will usually be 50 to 200 nucleotides in length, preferably from 80 to 120 nucleotides, although a oligonucleotide of any length may be appropriate in some circumstances. Suitable oligonucleotides may be prepared by the phosphoramidite method described by Beaucage and Carruthers, Tet. Lett. 22:1859-1862 (1981), or by the triester method, according to Matteucci et al. J. Am. Chem. Soc. 103:3185 (1981), or by other methods such as by using commercial automated oligonucleotide synthesizers.

The term “plasmid” refers to autonomously replicating, extrachromosomal circular DNA molecules, distinct from the normal bacterial genome and nonessential for cell survival under nonselective conditions. Some plasmids are capable of integrating into the host genome. A number of artificially constructed plasmids are used as cloning vectors.

The term “plurality” shall mean two or more.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

The term “probe” as used herein refers to an oligonucleotide whether occurring naturally or produced synthetically, which is either homologous or complementary to all or part of a nucleic acid sequence to be detected in, for example, a frozen cell sample, a tissue sample, or a standard. The probe is preferably selected so that under appropriate conditions it is capable of hybridizing specifically to nucleic acid sequences in a sample or standard.

The term “promoter” refers to a polynucleotide sequence that controls transcription of a gene or sequence to which it is operably linked. A promoter includes an RNA polymerase binding site and transcription initiation site. Generally, one selects a promoter known to be functional in the environment in which expression of the gene or sequence is contemplated. For example, if the expression environment is a cell, such as a bacterial or mammalian cell, one usually employs a bacterial or mammalian promoter. Alternatively, if the expression environment is in-vitro, the promoter is one that functions for the selected in-vitro polymerase activity.

A “recipient block” refers to a solid matrix for use in an array that has or is capable of having an array of wells defined therein for receiving samples of an array, including, but not limited to, cell suspensions, cell pellets, tissue cores, and internal standard preparations as described herein.

A “solid” phase or state shall mean one of the three fundamental states of matter, along with liquids or fluids and gases. Of these three states, the solid state has the greatest tendency to resist forces that would alter its shape; thus its shape and volume are fixed and are not affected by the space available to it.

The term “SQH₂O” refers to nuclease-free molecular biology-grade water.

The term “standard molecule” shall mean any biological molecule, as defined herein, and any other molecule, the known composition or concentration of which is used to analyze the presence, composition, structure and/or concentration of the another biological molecule in an array, such as in a tissue or frozen cell microarray. A standard molecule as used herein shall include, without limitation, polynucleotides, polypeptides, non-polypeptide hormones, cytokines, metabolites, metabolic precursors, drugs, as well as non-specific binders of probes, such as surgical dyes, bentonite, and cellulose. Where the standard molecule is a HER2-encoding polynucleotide, the polynucleotide comprises at least 20, 50, 100, or 200 or more contiguous nucleotides of the Her2 gene or its complementary sequence. Where the standard molecule is a VEGF-encoding polynucleotide, the polynucleotide comprises at least 20, 50, 100, or 200 or more contiguous nucleotides of the VEGF gene or its complementary sequence. Where the standard molecule is a HER2 polypeptide, the polypeptide comprises at least 10, 20, 50, or 100 or more contiguous amino acids of the HER2 polypeptide. Where the standard molecule is a VEGF polypeptide, the polypeptide comprises at least 10, 20, 50, or 100 or more contiguous amino acids of the VEGF polypeptide.

A “temperature-sensitive matrix material” shall mean a material that changes from a fluid or liquid state to a solid state as its temperature decreases below a freezing temperature. The freezing temperature may vary and is dependent upon the components of each specific temperature-sensitive matrix material. The freezing temperature of a temperature-sensitive matrix used in a frozen cell or tissue array is lower than or below the freezing temperature of the cells and/or tissue contained the array, such as, for example, 3° C., 5° C., 10° C. or more below the freezing temperature of the cells or tissue. Further, a temperature-sensitive matrix material facilitates cutting in its frozen state.

The term “tissue array” as used herein, and as further described herein, refers to a sectionable block, such as a paraffin block or frozen array block, that typically contains between one hundred to more than one thousand individual tissue samples as an array (rows and columns) of cores of biological tissue, each core having been punched from an individual donor tissue sample and embedded at a specific grid coordinate location in the sectionable block.

The terms “tissue microarray” and “TMA” are used interchangeably herein to refer to thin sections of a tissue array or frozen tissue array mounted on a planar platform or substrate, such as a microscope slide, such that the rows and columns of tissue or cells form a grid of samples (an array) on the platform. Tissue microarrays allow the examination of a large series of specimens while maximizing efficient utilization of technician time, reagents, and valuable tissue resources. Tissue microarrays can be used for rapid, large-scale screening of tissue expression patterns of potential therapeutic targets and studies of molecular markers associated with prognosis and response to therapy.

The term “transcription” shall mean synthesis of RNA by RNA polymerases using a DNA template.

The term “translation” shall mean the process in which the genetic code carried by mRNA directs the synthesis of proteins from amino acids.

The term “treated” with respect to a sample shall mean treatment of cells (such as in an animal, in a tissue of an animal, in a cell line, or in a cell suspension) that are subsequently used to prepare the sample by administering to the animal, the tissue, and/or the cells a treatment, such as a pharmaceutical drug or agent, or any other reagent of interest that may affect expression of a standard or biological molecule within a cell or tissue used to prepare a biological sample for an array.

### A Method and Apparatus for Making Frozen Arrays

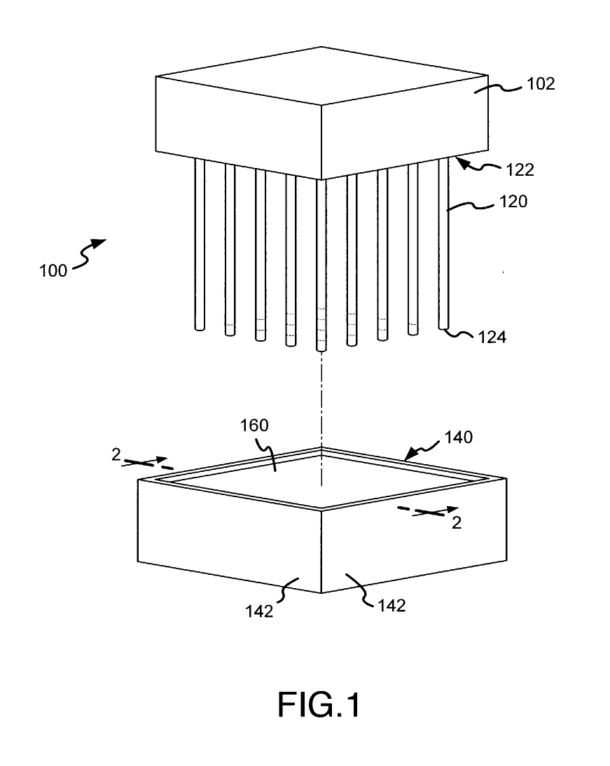

As shown in FIGS. 1-4, an arraying apparatus according to one embodiment of the invention comprises an arrayer 100 that is used to generate an array of wells in a mold 140 containing a temperature-sensitive matrix 160. As best seen in FIG. 1, the arrayer 100 includes a base 102 made of a rigid material, such as Plexiglas, plastic, ceramic, glass, metal, or wood and a plurality of pins 120 protruding from the base 102. Each of the pins 120 has a first end 122 within or affixed to the base 102 and a second free end 124. The pins 120 may be made of any type of material, including for example, hollow tubes with one or more blunt ends made of glass or metal (glass blunts) that are sealed, such as heat-sealed, and fixed, such as glued with epoxy, at or in the base 102. The free end 124 of each of the pins 120 is plugged with a sealer, such as metal, plastic, glue, adhesive, epoxy or other equivalent polymer. The pins 120 may be made of any rigid material that is capable of withstanding temperatures below 0° C., such as, metal, ceramic, and plastic. Further, the pins 120 may have hollow or solid lumens. The pins 120 can have a circular cross sectional shape, or any cross sectional shape conducive to creating a well to hold a biological sample, including, but not limited to, rectangular, oval, and the like.

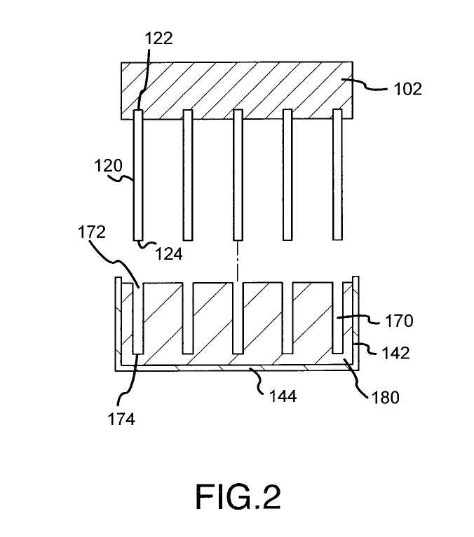

The mold 140 may be of any size and shape, including square, rectangle, oval, and the like, and may be sized and shaped so as to provide slices to fit appropriate analytical tools, such as microscope slides or trays. In one embodiment, the mold 140 is rectangular shaped and has four sides 142 and a bottom 144, as shown in FIG. 2.

The matrix 160 comprises a temperature-sensitive material that changes from a fluid or liquid state to solid state as the temperature decreases below a freezing temperature of the matrix material. Further, the temperature-sensitive matrix material facilitates cutting in its frozen or solid state. One useful temperature-sensitive matrix material comprises resin-polyvinyl alcohol and polyethylene glycol. Another useful temperature-sensitive matrix is optimal cutting temperature medium (“OCT medium”), which comprises resin-polyvinyl alcohol, an antifungal agent such as benzalkonium chloride, and polyethylene glycol for lowering the freezing temperature. OCT medium is commercially available, for example, Lab-Tek Instruments Co., Westmont Ill., manufactures OCT in three ranges of freezing temperature, −10° C. to −20° C., −20° C. to −35° C., and −35° C. to −50° C.

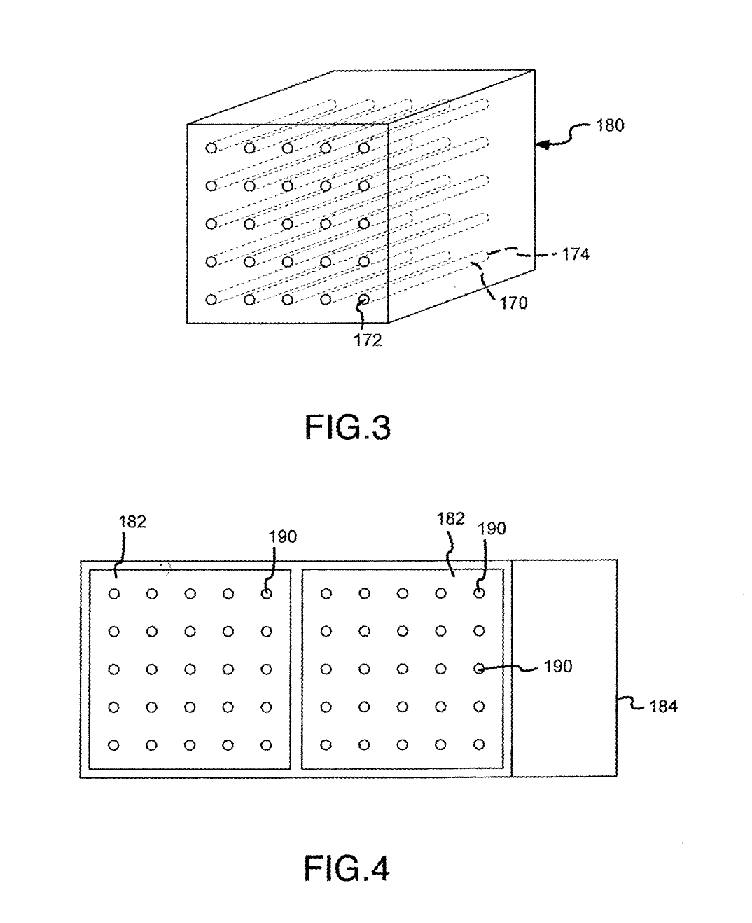

To make a frozen array, the pins 120 of the arrayer 100 can be first immersed in a lubricating material, such as glycerol, oil, fatty acids, grease, gel, fat, soap, and the like, and then partially immersed in the temperature-sensitive matrix 160, in its fluid state, and disposed in the mold 140, such that the free end 124 of the pins 120 does not touch the bottom 144 of the mold 140. While the arrayer pins 120 are engaged with the temperature-sensitive matrix 160 and the mold 140, the matrix 160 is frozen by lowering its temperature below the freezing temperature of the temperature-sensitive matrix material, such as at least 3° C., 5° C., 1020 C., or more. The mold 140 may be instantly frozen, for example, by submerging the mold 140 in a cryobath of isopentane. Alternatively, the mold 140 may be placed in a freezer, frozen in liquid nitrogen, or placed on dry ice. Using this method, the temperature-sensitive matrix 160 solidifies around the pins 120 in the mold 140. After the temperature-sensitive matrix 160 has frozen, the arrayer pins 120 are removed from the mold 140 to yield an array of wells 170 formed in an array recipient block 180 as shown in FIG. 3. The wells 170 in the frozen recipient block 180 correspond to the number and shape of the pins 120 and extend only partially through the matrix as shown in FIG. 2. Produced in this way, the frozen array recipient block 180 does not require a barrier material, such as glass or plastic tubes, to retain the samples. Instead, samples may be loaded directly into the wells 170 formed in the frozen recipient block 180, thereby making it easier to produce and slice the array. The array recipient block 180 is removed from the mold 140 prior to slicing. After removal of the mold 140, each of the wells 170 has a first open end 172 where one of the pins 120 entered and exited the matrix 160 and a second closed end 174 within the matrix 160. The recipient block 180 is stored at a temperature sufficient to maintain the temperature-sensitive matrix 160 in a frozen solid state until one or more samples are loaded into the wells 170. In alternative embodiments, the frozen recipient block is stored at a temperature at least 3 C. below the freezing temperature of the temperature-sensitive matrix, alternatively at least 5 C. below, or at least 10° C. below the freezing temperature of the temperature-sensitive matrix.

One or more samples, such as cell suspensions, cell pellets, or tissue cores, are inserted directly into the wells 170 of the frozen recipient block 180 to form a frozen array of samples. When biological samples, such as cell suspensions, are inserted directly into the wells 170 of the frozen recipient block 180 the cells instantly freeze within the well 180. In this way, the cells are preserved without requiring fixation, preservatives, or other type of chemical treatment. With respect to tissue samples, an arraying instrument, such as Beecher Instrument, is used to punch cores from tissue samples, for example, tissue that has been flash frozen using liquid nitrogen. Alternatively, tissue cores may be punched manually from donor blocks. These frozen tissue cores are inserted into the wells 170 using a similar arraying instrument. The frozen recipient block 180 likewise maintains the freezing temperature of the tissue cores, thus enabling the tissue to be inserted into the array recipient block 180 without fixation or other type of chemical treatment.

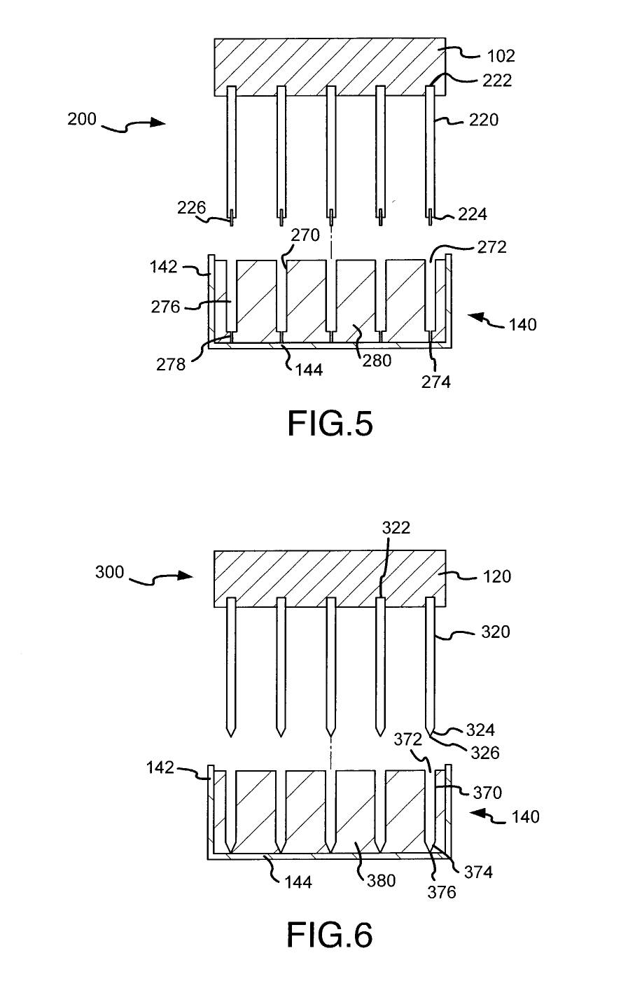

The recipient block 180 containing samples can be sliced horizontally perpendicular to the longitudinal axis of the wells 170 to form one or more frozen array slices 182 that are then applied to a microscope slide 184 or other analytical platform as shown in FIG. 4 to form frozen cell or tissue microarrays. Each array slice has a spot (or transverse section) 190 of sample corresponding to the sample contained within each of the wells 170 of the array before it was sliced. One or a plurality of the array slices 182 may be placed on each of the slides 184. The slides 184 likewise may be stored at freezing temperature until used. For analysis, the slides can be treated to remove the matrix material and thereby form a microarray of spots on the microscope slide. The matrix may be removed using various types of chemicals, such as for example, aqueous buffers, xylene, and acetone. Merely allowing the slide 184 to sit at room temperature will cause the temperature-sensitive matrix, for example, OCT, to melt making it easier to be removed from the slide 184. Virtually any kind of analytical procedure or molecular analysis that can be performed on a microscope slide can be performed on the microarray made from a frozen array, including, but not limited to, in-situ hybridization, immunochemistry, PCR, and ligand/receptor binding procedures.

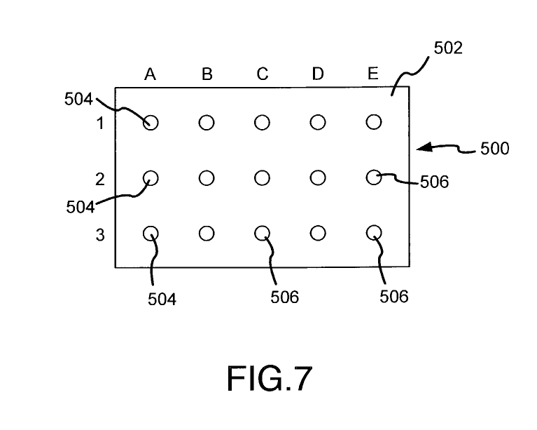

As shown in FIG. 5, another embodiment of the invention includes an arrayer 200 having a plurality of pins 220 affixed to or within a base 102. The pins 220 each have a first end 222 at or within the base 102 and a second free end 224. Like the pins 120, shown in FIG. 1, the pins 220 may be made of glass blunts that are heat-sealed and glued with epoxy in the base 102. The free end 224 of each of the pins 220 is plugged with a sealer, such as metal pieces and epoxy. In contrast to the pins 120, the pins 220 have a smaller elongated appendage, such as a needle 226, submerged within the sealer and extending beyond the free end 224. As described above, the pins 220 are used to create a plurality of wells 270 in an array recipient block 280. Here however, the pins 220 are fully inserted into the temperature-sensitive matrix 160, in its fluid state, in the mold 140 such that the free end 224 of the pins 220 touches the bottom 144 of the mold 140. Using this procedure, the pins 220 create an array of wells 270 that extend through the entire recipient block 280 and have two open ends 272 and 274 when the array recipient block 280 is removed from the mold 140. In this way, a solid sample, such as a tissue core, can be inserted through the open end 272 of the well 270 and the opposite open end 274 will provide a pathway, for example, for air within the well to escape, thereby relieving some pressure and making it easier to insert a solid sample into the well 270. Further, the wells 270 have two portions 276 and 278, each having a diameter corresponding to the diameter of the pins 220 and the needles 226, respectively. The smaller size of the second portion 278 provides a stop mechanism for a solid core when the solid core is inserted within the well 270.

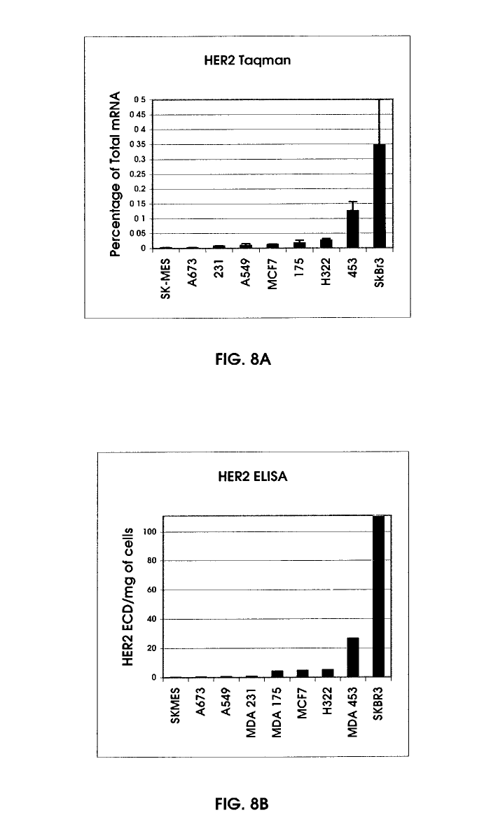

FIG. 6 shows further embodiment of the invention. An arrayer 300 has a plurality of pins 320 affixed to or within a base 102. The pins 320 each have a first end 322 at or within the base 102 and a second free end 324. The pins 320 may be made of any solid material, such as metal or glass. The free end 324 of each of the pins 320 tapers to form a point 326. As described above, the pins 320 create a plurality of wells 370 in an array recipient block 380. The pins 320 are fully inserted into the temperature-sensitive matrix 160, in its fluid state, in the mold 140 such that the free end 324 of the pins 320 touches the bottom 144 of the mold 140. Using this procedure, the pins 320 create an array of wells 370 that extend through the entire recipient block 380 and have two open ends 372 and 374 when the recipient block 380 is removed from the mold 140. The open end 374 of the well 370 has an small opening 376 corresponding to the size of the point 326 of the pin 320. The opening 376 provides a pathway for air within the well 370 to escape when a solid sample, such as a frozen tissue core, is inserted into the well 370.

### Internal Standards in Arrays

In one embodiment of the invention, one or more internal standard preparations may be used for detecting or quantitating selected molecules, including biological molecules such as nucleic acids, polypeptides, proteins, and antibodies, or other compounds, such as in a cell or tissue, in an array, such as a tissue microarray, a cell array, or a frozen tissue or cell array as discussed herein. The internal standard preparation comprises a known quantity of a standard molecule, such as a biological molecule, incorporated into an embedding material to form an internal standard preparation that can be inserted into a well of an array. The internal standard preparation may contain in-vitro translated proteins, in-vitro transcribed RNA, plasmid or PCR-amplified DNA, cell homogenates, along with carrier proteins, such as bovine serum albumin (BSA), polycations such as protamine, spermine or spermidine, or any other substance that aids in quantitating a biological or standard molecule in an array.

An array and microarray utilizing internal standard preparations are made as follows. One or more internal standard preparations are inserted into one or more of a plurality of wells disposed within an array recipient block. Samples, such as tissue, cell suspensions, or cell pellets are inserted into other wells of the array recipient block to form an array. The array is sliced and one or more of the array slices are placed, for example, on a microscope slide for analysis. The matrix material may be removed from the microarray or array slides using various techniques and/or chemicals, including aqueous buffers, xylene, citruline, alcohols, or other organic solvents, liquid CO₂ (in critical point drying), or evaporation. The internal standard preparation allows a standard molecule to be retained on an array slide throughout processing, such as removal of the matrix material, and analytical procedures performed on the array slide. Because matrix material is removed throughout processing of an array slide, the embedding material must differ from the matrix material in at least one physical or chemical property, including for example, solubility, temperature sensitivity (such as freezing temperature, melting temperature and the like), pH, or affinity for the planar substrate used to prepare a microarray.

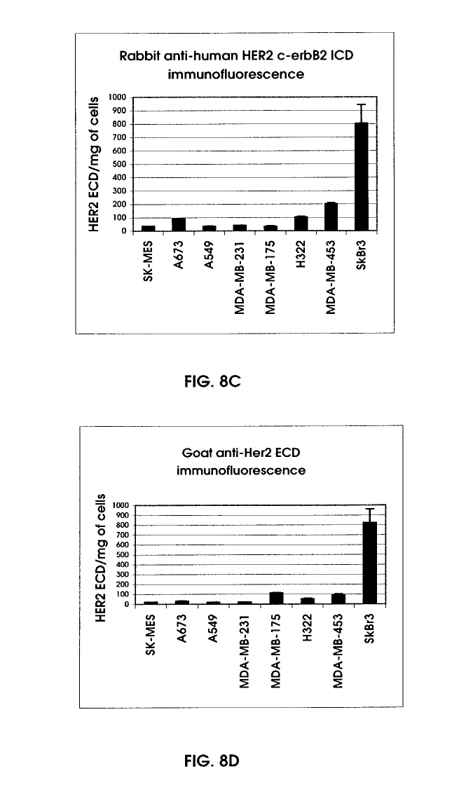

FIG. 7 shows a microarray 500 utilizing internal standard preparations. The microarray 500 is mounted on a microscope slide 502 and has an array of spots (or transverse sections) of sample or internal standard preparation organized in five rows, designated 1-5, and five columns, designated A-F. Five spots of internal standard preparation 504 occupy positions A1-A5 of the microarray 500. Twenty spots of tissue sample 506 occupy the remaining positions.

Various analytical procedures or molecular analyses may be performed on array slides, for example, to detect or test for the presence of a standard molecule, including for example, in-situ hybridization, immunohistochemistry, and the like. In conjunction with these analytical procedures, a detectably labeled compound, such as a probe or polypeptide bearing a detectable signal or label, such as a luminescent label, a fluroescent label, a radiolabei, and the like, is used. Examples of detectably labeled compounds include a labeled probe, a labeled polypeptide, such as a monoclonal antibody, an antibody binding fragment, a receptor, a receptor ECD, or a ligand binding fragment of a receptor, and a binding protein, such as an antibody antigen, a receptor ligand, biotin, or streptravidin. Various equipment and methods can then be used to detect a label, such as using a phosphorimager or a CCD camera or other imaging device to record a luminescent label over an entire array slide. Because a quantity of the standard molecule in the internal standard preparation is known, a quantitative signal or result obtained on analysis of the internal standard preparation can be correlated with a signal or result obtained on analysis of the samples to determine an amount of the standard molecule present in the sample.

Additionally, an internal standard preparation may also act as a positive control. For example, if a sample fails to show a positive result, the integrity of the analytical procedure can be analyzed relative to a positive result obtained for an internal standard preparation. Negative controls, such as internal standards without the standard molecule or internal standards containing molecules otherwise expected to show a negative result, may also be incorporated into other wells of an array recipient block.

In one embodiment, a biological molecule such as synthetic RNA may be used in an internal standard preparation. A cloned DNA sequence can be used to generate a synthetic RNA internal standard preparation. For instance, it is thought that there are more than 30,000 human genes, including, for example, human VEGF, (Leung, D. W., et al. Science 246, 1306-1309 (1989)), Her2/ErbB2, (Coussens, L., et al.. Science 230, 1132-1139 (1985)) cytoplasmic actin, and glyceraldehyde dehydrogenase, and the like. RNA transcripts of these genes, or fragments thereof, as well as others to be described in the future, can be synthesized, incorporated into an embedding material, and used as an internal standard preparation in an array that is designed to analyze expression of one or more of these genes.

The arrays and internal standard preparations described herein can be used in known methods and procedures for the analysis of cellular biomolecules, for example, to characterize a tissue-specific expression (i.e. measure tissue mRNA content) of a gene represented by a novel cDNA sequence. Using standard techniques, the cDNA can be used to generate a synthetic sense-orientation RNA strand. This RNA strand can be incorporated into a solid embedding material, such as agarose, polyacrylamide, gelatin, or coagulated (denatured) protein such as BSA, and used as a “target” or an internal standard in parallel with samples, such as various tissues of interest, in an array. That is, the array contains the RNA internal standard preparation in least one well and one or more samples occupy other wells of the array. The entire array is sliced, and the slices are placed, for example, on a microscope slide and probed under suitable hybridization conditions with a molecular probe, such as an anti-sense RNA or DNA sequence that is homologous to the RNA standard and is labeled to allow later detection. For example, the RNA or DNA probe might contain a radioactive isotope or a luminescent label that would allow detection by film or phosphorimager. Or, the RNA or DNA probe might contain an antigen such as digoxigenin, biotin, or FITC (fluorescein isothiocyanate) that could be detected indirectly with appropriate antibodies or proteins (such as streptavidin) coupled to enzymes or other markers which, using standard techniques, could reveal the location of the probe. Alternatively, the RNA or DNA probe comprises a sequence and is subsequently hybridized to a labeled probe.

Binding of an appropriate probe to the samples of tissue is in proportion to the amount of sense mRNA present in the tissue. An anti-sense probe also binds the sense RNA contained in the internal standard preparation. Because the quantity of RNA in the internal standard preparation is known, the amount of mRNA in the sample can be determined by correlating the signal intensity of the internal standard preparation with the signal intensity of the sample. Numerical values for the expression (the amount of detectable label) may be obtained in a number of ways, including for example, by using a phosphorimager, CCD camera, or other electronic imaging systems to detect luminescent labels, such as chemiluminescent, fluorescent, or radioactive signals. Typically, these systems generate electronic image files that can be analyzed and quantitated using a variety of software tools including, for example, Adobe Photoshop, Scion IMAGE, NIH IMAGE, and Phoretix Array². Indeed, using this method makes it possible to determine a specific numeric amount of the mRNA in the sample, such as molecules per unit volume of mRNA in the tissue sample.

In a well-controlled procedure, the amount of probe bound to a tissue is one measure of the level of expression of the corresponding gene in that tissue. However, an analytical procedure showing no detectable probe binding to any tissue may be difficult to interpret without appropriate positive control samples. If, however, the internal standard preparation described above is analyzed in parallel with the sample tissue and does bind appropriately to the anti-sense RNA probe, the technical integrity of the analytical procedure can be confirmed. In other words, a negative result in the tissue samples can be interpreted as a true negative, rather than as a technical procedural failure, when the control internal standard preparation shows a positive result. Of course, this interpretation depends on the investigator also knowing that the tissue samples were well-preserved and contained mRNA capable of hybridizing to the RNA probe. The integrity of the samples may be determined by performing parallel analytical procedures on duplicate slides containing slices from the same array, and using probes homologous to well-known abundant and widely expressed genes such as: cytoplasmic beta-actin and glyceraldehyde dehydrogenase, and the like. If the well-known anti-sense probes bind to the tissue samples, and the corresponding sense probes do not, the integrity of the tissue samples can be confirmed.

Internal standards for use in arrays are not limited to RNA or other polynucleotides, but can be prepared with any biological or standard molecule, including, but not limited to, DNA and polypeptides or proteins. For example, an investigator may wish to evaluate expression of a specific protein in a tissue sample by incorporating as a control an in-vitro synthesized protein sample or a natural protein sample into an embedding material, such as agarose, to create a protein internal standard preparation. The standard protein could be detected with a specific reagent such as an detectably labeled monoclonal or polyclonal antibody, receptor or receptor ligand. The protein internal standard preparation can be used in parallel with various tissue samples as a target to which the antibody is reacted. That is, one or more of a plurality of wells disposed within an array contain the protein internal standard preparation and one or more of the plurality of wells disposed within the array contain tissue samples. In this way, the internal standard preparation allows quantitation of protein expression in the sample and also acts as a positive control for procedural integrity. Protein expression in a tissue sample may be quantitated by correlating results of an antibody reaction in the internal standard preparation with results of an antibody reaction in the sample. Further, if the antibody reacts with standard protein of the protein internal standard preparation, the procedural integrity of the antibody staining reaction is confirmed, even if no tissue sample reacts with the antibody.

The above-described internal standard preparations can be employed to determine a quantity of any selected molecule in any sample. Samples used in an array can consist of any sample of interest, including for example, normal tissue, diseased tissue, inflamed tissue, tumors, tissue at various stages of development, where the cells have been treated with various reagents that may affect expression of a selected molecule, cell suspensions, and cell pellets. Standard molecules may include, but are not limited to, different orientations (sense or anti-sense) or splice variants of polynucleotides, such as RNA or DNA, and/or different isoforms of proteins (full-length or partial sequences). Internal standard preparations can contain more than one standard molecule, including, but not limited to, multiple kinds of polynucleotides and/or multiple kinds of polypeptides.

The biological or standard molecule of the internal standard can be embedded in any embedding material that: (1) will allow the standard molecule to be inserted into a well of an array; and (2) will retain the standard molecule in the array or on an array slide throughout processing and analytical procedures performed on the array or the array slide. Embedding materials can include for example, agarose, alone, or in combination with BSA, and/or carrier proteins to help prevent a standard molecule from diffusing out of an internal standard preparation. Additionally, a material that provides an envelope for a standard molecule may be included in an internal standard preparation for preventing diffusion of the standard molecule into the matrix of an array. For example, red blood cell ghosts or liposomes can act as an envelope for RNA to prevent RNA molecules from diffusing into the matrix of an array. An appropriate choice of fixatives will minimize RNA diffusion out of the standard sample during preparation. The concentration and materials of the embedding material should be chosen to allow the internal standard preparation to form a solid or gel-like state. However, care should be taken to choose a material and concentration, for example, of agarose that prevents the internal standard preparation from becoming too rigid, which may inhibit the ability to remove an internal standard core from a donor block and insert the internal standard preparation into a well of an array. Additionally, an internal standard preparation that is too rigid may make it more difficult to slice the array. Examples of embedding materials that form solid or gel-like states include for example, about 1-3% agarose and in a range of about 1-20% BSA. Alternatively, the embedding material can contain about 2% agarose and about 1-5% BSA. A concentration of about 2% agarose without BSA works well to form a solid or gel-like state without becoming overly rigid.

In one embodiment, internal standard preparations are generally made by isolating one or more biological molecules, mixing the biological molecules with an embedding material, and preparing the mixture for insertion into an array. The internal standard preparations may be inserted into an array in a number of ways. For example, the internal standard preparation may be poured into a mold and allowed to solidify or form a gel donor block. The internal standard donor block is removed from the mold and cores from the internal standard donor block are taken with a typical arraying instrument, such as Beecher instrument or punch. The cores of the internal standard preparation donor block may then be inserted into the wells of any array recipient block using a standard arraying instrument. Alternatively, the internal standard preparation may be inserted into the well of the array recipient block in a fluid state, e.g., before it has had a chance to gel, using any type of needle, syringe, or funnel. In this case, the internal standard preparation will form a solid or gel in the well of the recipient array block. In the case of a frozen array, a fluid internal standard preparation is inserted directly into a well of a frozen array recipient block and freezes within the well.

In another embodiment of the invention, multiple different internal standard preparations may be included in an array. For example, each of the multiple different internal standard preparations may contain different concentrations of the same biological molecule for creating a standard curve of concentrations. Specifically, the array may include multiple cores of internal standard preparations having a standard curve range of concentrations of the biological molecule to assess qualitatively or quantitatively the level of detection of the biological molecule in an array of samples of tissue or cell lines. In this embodiment, the internal standards are prepared as described above, except that multiple mixtures and/or donor blocks are made, each having a different concentration of the biological molecule.

In another embodiment of the invention, a universal internal standard preparation is made using multiple different biological molecules in the same internal standard preparation, including, but not limited to, multiple types of polynucleotides and multiple types of polypeptides. This universal internal standard preparation may be used for many types of analytical procedures seeking to detect and/or quantify multiple types of biological molecules in an array.

In another embodiment, an internal standard preparation may be used as an orientation marker in an array. For orientation with light microscopy, a colored surgical dye is admixed with an embedding material and inserted into an array as described above. For orientation on a phosphorimager, for example, a standard molecule, such as a non-specific binder of a biological molecule, is admixed with an embedding material and inserted into an array as described above. Non-specific binders include bentonite and cellulose. The non-specific binders will bind any probe used in an analytical procedure performed on the array and thus generate a positive result for the spot containing the orientation marker. The internal standard preparation orientation marker can be placed in one or more wells located in strategic positions, such as in an asymmetric pattern at one side of an array, throughout the array to provide a guide or map to indicate array orientation when reviewing results of an analytical procedure performed on an array slide.

### EXAMPLES

The following examples are intended to illustrate but not limit the invention. While they are typical of those that might be used, other procedures are known to those of skill in the art and may alternatively be used.

### Example 1

### RNA/Agrose Internal Standard Preparation

The present example demonstrates the utility of the invention for analyzing biological molecules, such as RNA, in a cell or tissue array using an internal standard having a known quantity of the biological molecule in a solid embedding material that differs from a matrix material used to make the array. Specifically, the present example demonstrates an approach for embedding a specific RNA molecule in agarose and BSA to form an internal standard preparation for use in an array so that the RNA molecule is retained throughout processing and analytical procedures performed on the array. The embedded RNA can be used simply as a positive control for procedural success, as a component in a basic assay to improve upon procedural methods, or ultimately as a quantitative standard to assess comparative levels of gene expression in tissues or cells.

### Preparation of Biological Molecule

RNA, for example human Her2/c-ErbB2, was transcribed in-vitro using the following procedure, which procedure was described in detail in Lu L H, Gillett N A. Cell Vision 1:169-176 (1994), an optimized protocol for in-situ hybridization using PCR-generated ³³P-labeled probes. Alternatively, an Ambion Maxiscript or Ambion Megascript kit may be used to perform this procedure (Ambion, Austin, Tex.). First, the following were added to a siliconized 1.5 ml microfuge (Eppendorf) tube: 1 μg of linear double-stranded DNA template encoding the human Her2/ErbB2 gene (Coussens, L., et al., Science 230: 1132-1139 (1985)) (Genentech, South San Francisco, Calif.) comprising a PCR-amplified cDNA fragment flanked by RNA polymerase promoter sequences (e.g. bacteriophage T3 or T7 promoters); 2 μl of 10×Reaction Buffer; 8 μl High-Concentration rNTPs; 2 μl T3 or T7 polymerase enzyme mix depending upon which promoter was used; and nuclease-free water to final volume of 20 μl. The mixture was incubated for 4 hours at 37° C. to synthesize the synthetic RNA. Alternatively, the DNA template could comprise a linearized plasmid DNA encoding the desired sequence flanked by RNA polymerase (e.g. bacteriophage T3 and/or T7) promoters. Next, 1 μl of DNase (Ambion) was added to the Eppendorf tube containing the synthesized RNA and the mixture was incubated for 15 minutes at 37° C. This step degraded the DNA template in the reaction, so that it could be removed later. To stop the degradation reaction, 80 μl TE was added to the Eppendorf tube. An RNeasy Mini Kit (Qiagen, Germantown, Md.) was used to purify the RNA transcript in the RNA solution. A spectrophotometer was used to determine the concentration of the RNA transcript in the RNA solution. Next, 1 μg of this RNA solution was analyzed on a 6% Polyacrylamide TBE/Urea gel (Invitrogen, Carlsbad, Calif.) to confirm that the transcript was of the proper length. The Her2/ErbB2 RNA solution was stored at −20° C. until ready to use.

### Preparation of Internal Standard

An internal standard preparation was prepared according to the following methods using the Her2/ErbB2 RNA solution prepared as described above. A working concentration of 100 ng/μl of the RNA solution was made. An aliquot of 50 μl of the RNA solution (5 μg) was added to 200 μl of TE in a new Eppendorf tube. The Eppendorf tube was heated in a 95° C. heat block for 3 minutes to denature the RNA transcript and then chilled immediately on ice. To the RNA solution, 250 μl of 8% NuSieve 3:1 (a high gel strength agarose melted at 99° C.) (FMC Bioproducts, Rockland, Me.) and 500 μl SQH₂O that had been warmed in a 50° C. heat block were added. The resulting RNA/agarose mixture was vortexed briefly and then poured into a 15 mm×15 mm DisPO base mold (Baxter Scientific, McGaw Park, Ill.). The final concentration of the RNA was 5 μg/ml. The RNA/agarose internal standard preparation was then allowed to gel at 4° C. for at least one hour. To vary the concentration of either RNA or agarose, the volume of either can be increased with a reciprocal reduction in the amount of SQH₂O.

As desired, a carrier protein such as bovine serum albumin (BSA) or other component such as protamine, polyinosine, spermidine, or in-vitro translated proteins can be incorporated into the agarose blocks by adding the desired amount and adjusting the volume of SQH₂O accordingly, to obtain a final volume, for example, of 1 ml. For instance, BSA (Roche, Indianapolis, Ind.) was made as a 10% stock solution in water and heated at 50° C. to solubilize before being mixed with the RNA/agarose to achieve a desired concentration. To create an internal standard block containing 5% BSA, 500 μl of SQH2O referenced above was replaced with 500 μl of 10% BSA to create an internal standard preparation containing 2% agarose and 5% BSA.

After the gel was formed, the RNA/agarose blocks were removed from the plastic molds, using a clean razor blade, and the intact block was fixed in 10% neutral buffered formalin (Richard Allen Scientific, Kalamazoo, Mich.). Some of the RNA fixed in neutral buffered formalin diffuses out of the standard matrix during the fixation process. To prevent this diffusion, alternative fixation methods can be used. For example, the intact block of RNA/agarose was fixed in a precipitating fixative containing 0.5 M sodium acetate, pH 5, 70% ethanol and 20% (v/v) of stock 37% formaldehyde (final concentration of formaldehyde is 7.4%) overnight at room temperature. The agarose block was then transferred to 70% ethanol (in water) and processed (according to standard techniques) for paraffin embedding. The samples were incubated in 70% FLEX alcohol (Richard Allen Scientific, Kalamazoo, Mich.) for 30 minutes, then twice in 95% FLEX alcohol (Richard Allen Scientific, Kalamazoo, Mich.) for 1 hour each, then three times in 100% FLEX alcohol (Richard Allen Scientific, Kalamazoo, Mich.) for 30 minutes each, then three times in Clearing Agent (Richard Allen Scientific, Kalamazoo, Mich.) for 45 minutes each and finally incubated for 30 minutes at 60° C. in 100% molten paraffin for 30 minutes each. Samples were then embedded into paraffin using a Leica histoembedder (Leica, Deerfield, Ill.).

These methods can also be used with the frozen array embodiment described herein to create frozen samples of RNA internal standard preparations for use in frozen array recipient blocks, for example, by eliminating the foregoing fixation steps.

### Example 2

### Protein/Agrose Internal Standard Preparation

The present example demonstrates the utility of the invention for analyzing biological molecules, such as proteins, in a cell or tissue array using an internal standard having a known quantity of the biological molecule in a solid embedding material that differs from a matrix material used to make the array. Specifically, the present example demonstrates an approach for embedding a specific protein molecule in agarose to form an internal standard preparation for use in an array so that the protein is retained throughout processing and analytical procedures performed on the array. The embedded protein can be used simply as a positive control for procedural success, as a component in a basic assay to improve upon procedural methods, or ultimately as a quantitative standard to assess comparative levels of protein expression in tissues or cells.

A final concentration of 0.45 mg/mL of Her2/ErbB2 ECD protein (Molecular Oncology, Genentech, South San Francisco, Calif.) was made by adding 500 μl of 1.09 mg/mL of synthetic Her2/ErbB2 extra-cellular domain protein and 250 μl of SQH₂O to an Eppendorf tube. The protein/water mixture was vortexed briefly. Next, 250 μl of 8% NuSieve 3:1 (a high gel strength agarose melted at 99° C.) that had been cooled briefly to approximately 60° C. was added to the protein/water mixture and then vortexed briefly. To vary the concentration of either protein or agarose, the volume of either can be increased with a reciprocal reduction in the amount of SQH₂O. As desired, a carrier protein such as BSA or other component such as naturally occurring or synthetic peptide sequences or naturally occurring or in-vitro translated proteins can be incorporated into the agarose blocks by adding the desired amount of carrier protein and adjusting the volume of SQH₂O accordingly, to obtain a final volume, for example, of 1 ml.

The protein/agarose internal standard preparation was then poured into a 15 mm×15 mm DisPO base mold (Baxter Scientific) and allowed to gel at 4° C. for at least one hour. The protein/agarose blocks were removed from the plastic molds using a clean razor blade, and then the intact block was fixed in 10% neutral-buffered formalin overnight at room temperature. The fixed protein/agarose block was then transferred to 70% ethanol in water and processed according to standard techniques for paraffin embedding in an array as described in Example 1.

This method can also be used in the frozen array system described herein to create frozen samples of protein for use in preparing frozen internal standard preparations by eliminating the fixation step.

### Example 3

### In -situ Hybridization on Internal Standard Preparations in a Tissue Microarray

The present example demonstrates the utility of the invention to serve as internal standards and controls in a tissue microarray (TMA) histological section for in-situ hybridization procedures. Specifically, the present example demonstrates the utility of the present invention to quantitate VEGF A mRNA expression in colon tumors in an in-situ hybridization procedure. Further, the present example demonstrates the utility of the invention for quantitating biologically useful molecules, such as RNA, in an array using a multiple different internal standards, each having a different quantity of the biological molecule to set up a standard curve of expression signals.

A colon tumor tissue microarray was constructed containing 282 cores arrayed in 20 columns and 15 rows as follows. One hundred seventy seven cores of sample, measuring 0.6 mm in diameter, were taken from various donor paraffin blocks including 44 specimens of colonic adenocarcinoma (National Cancer Institute Cooperative Human Tissue Network (CHTN), Western Division, Vanderbilt University Medical Center, Nashville, Tenn.; see http://www-chtn.ims.nci.nih.gov/), 6 specimens of normal colon adjacent to tumor (CHTN), 6 specimens of colonic adenocarcinoma metastatic to liver (CHTN (1 case); University of Glasgow (1 case), Glasgow, Scotland; University of Michigan (4 cases); Ann Arbor, Mich.), and 6 cases of benign colonic adenoma (CHTN) (usually in duplicate or triplicate). The sample cores were next embedded into a recipient paraffin block by, for example, using a Beecher tissue arraying instrument (Beecher Instruments, Silver Spring, Md.) as described herein.

Twelve cores of internal standards, measuring 0.6 mm in diameter, were taken from six donor blocks containing three different concentrations of RNA/agarose internal standard preparations, prepared as described in Example 1 above. Half of the RNA/agarose standards contained anti-sense RNA for human VEGF A (Leung, D. W., et al., Science 246: 1306-1309 (1989)) at 0.5, 1.0, and 5.0 μg/ml (all in duplicate) and half of the standards contained sense RNA for human VEGF A at 0.5, 1.0, and 5.0 mg/ml (all in duplicate). The RNAs were synthesized by in-vitro transcription from the PCR-amplified sequence described below. The internal standard preparation cores were embedded in the paraffin recipient block in the same manner as the sample cores described above.

Four cores, measuring 0.6 mm in diameter, from 2 different human xenograft tumor cell lines (COLO205 (ATCC catalog number CCL-222) and HCT116 (ATCC catalog number CCL-247)) were embedded in the recipient paraffin block using a similar method. Eleven cores, measuring 0.6 mm in diameter, of 8% NuSieve 3:1 agarose and 50% blue, yellow, or black surgical marking dye prepared as described in Example 15 (Triangle Biomedical S, Durham, N.C.) which are useful for orientation during evaluation of the section, were embedded in the recipient paraffin block.

All of the cores were annealed in the recipient paraffin block array by incubating the block in a 37° C. oven overnight. The paraffin array was sliced into two or more 3-5 μm thick histological TMA sections, each TMA section having an array of spots corresponding to the array of cores in the recipient paraffin block. Each TMA section was then transferred into a 42° C. water bath and then collected individually onto glass slides and allowed to dry thoroughly.

In-situ-hybridization analysis was performed on some of the colon tumor TMA slides. The TMA slides were hybridized to human VEGF A sense and anti-sense RNA probes using the following techniques. The sequence for the PCR-amplified DNA template used to transcribe the sense and anti-sense probes was:

| | |
|---|---|
| GGGCCTCCGAAACCATGAACTTTCTGCTGTCTTGGGTGCATTGGAGCCTCGCC | [SEQ ID NO: 1] |
| TTGCTGCTCTACCTCCACCATGCCAAGTGGTCCCAGGCTGCACCCATGGCAGA | |
| AGGAGGAGGGCAGAATCATCACGAAGTGGTGAAGTTCATGGATGTCTATCAGC | |
| GCAGCTACTGCCATCCAATCGAGACCCTGGTGGACATCTTCCAGGAGTACCCT | |
| GATGAGATCGAGTACATCTTCAAGCCATCCTGTGTGCCCCTGATGCGATGCGG | |
| GGGCTGCTGCAATGACGAAGGCCTGGAGTGTGTGCCCACTGAGGAGTCCAACA | |
| TCACCATGCAGATTATGCGGATCAAACCTCACCAAGGCCAGCACATAGGAGAG | |
| ATGAGCTTCCTACAGCACAACAAATGTGAATGCAGACCAAAGAAAGATAGAGC | |
| AAGACAAGAAAATCCCTGTGGGCCTTGCTCAGAGCGGAGAAAGCATTTGTTTG | |
| TACAAGATCCGCAGACGTGTAAATGTTCCTGCAAAAACACAGACTCGCGTTGC | |
| AAGGCGAGGCAGCTTGAGTTAAACGAACGTACTTGCAGATGTGACAAGCCGAG | |
| GCGGTGAGCCGGGCAGGAGGA | |

The TMA slides were baked in an oven to adhere tissue to glass at 37° C. overnight followed by 65° C. for 30 minutes. The sections were deparaffinized in a Leica Autostainer XL (Leica, Deerfield, Ill.) by incubating 3 times for 5 minutes each in Xylenes (Richard Allen, Kalamazoo, Mich.) then rehydrating through a graded ethanol series to distilled water. Slides were then washed twice in 2×SSC (0.3 M NaCl, 0.030 M NaCitrate, pH 7.0) for 5 minutes each time. The slides were treated for 15 minutes in a 20 μg/ml Proteinase K (Roche Diagnostics, Indianapolis, Ind.) in 10 mM Tris pH 8.0/0.5 M NaCl solution at 37° C. and washed for 10 minutes in 0.5×SSC (0.075 M NaCl, 0.007 M NaCitrate, pH 7.0). The slides were dehydrated with an ethanol gradient (70%-95%-100%) and air-dried. The slides were covered with 100 μl hybridization buffer (50% formamide, 10% dextran sulfate, and 2×SSC) and prehybridized for 1-4 hours at 42° C. The [³³P]-labeled single-stranded VEGF A RNA probe (anti-sense orientation) referenced above, at a concentration of 2×10⁶ cpm, was dissolved in 100 μl of hybridization buffer containing 1 mg/ml tRNA and added to the prehybridization buffer on one of the slides, mixed well, covered with coverslip, and allowed to hybridize overnight at 55° C. in a sealed humidified container.

The foregoing hybridization procedure was performed on another slide from the TMA using a [³³P]-labeled single-stranded VEGF A RNA probe transcribed from the same PCR-amplified template described above, but in the sense orientation.

After hybridization, the slides were washed twice for 10 minutes in 2×SSC containing 1 mM EDTA at room temperature, and then incubated for 30 minutes at 37° C. in 20 μg/mL RNase A in 10 mM Tris pH 8, 0.5 M NaCl. The slides were washed for 10 minutes in 2×SSC containing 1 mM EDTA at room temperature, then washed 4 times for 30 minutes each in 0.1×SSC containing 1 mM EDTA at 55° C., and then washed in 0.5×SSC for 10 minutes at room temperature. The slides were dehydrated for 2 minutes each in 50%, 70%, and 90% ethanol containing 0.3M ammonium acetate, and allowed to dry in the air.

In order to view the results of the hybridization, the slides were exposed to a storage phosphor screen (Kodak) for 18 hours and then the phosphor screen was scanned with a Typhoon 8600 variable mode imager (Molecular Dynamics, Sunnyvale, Calif.). The image was quantified using Phoretix Array² (Nonlinear USA Inc, Durham, N.C.) software and data was analyzed using Microsoft Excel.

The amount of VEGF A RNA present in the internal standard preparations and in the samples was calculated as follows. First, the amount of VEGF A RNA in the RNA/agarose standards was calculated. The VEGF A RNA used in the RNA/agarose standard was approximately 604 bases long. Each base was assumed to weigh 340 daltons, and thus each molecule of VEGF A RNA weighed approximately604×340 daltons or approximately 2.05×10⁵ daltons. The RNA/agarose standard contained 0.5 μg/ml of VEGF A RNA and therefore contained approximately 2.43×10⁻¹² moles/ml, or approximately 1.5×10¹² molecules/ml, or approximately 1.5 molecules/μm³ of VEGF A RNA. A histological section 5 μm thick of the RNA/agarose standard therefore contained approximately 7.5 molecules of VEGF A RNA per square micron.

To determine the amount of VEGF A RNA in the samples, the intensity of the signal from the autoradiographic film or phosphoroimager analysis of the samples was correlated to the intensity of the signal from the RNA/agarose standard, which gave a quantity of VEGF A RNA in the samples expressed in molecules per unit volume of tissue. Table 1 summarizes the data obtained from the above-described in-situ hybridization using the anti-sense probe.

TABLE 1

| | PHOSPHOR- | QUANTITY OF |
|---|---|---|
| | IMAGER | VEGF A RNA |
| CONTENT OF CORE/TMA SPOT | SIGNAL* | (molecules/μm³) |
| VEGF A RNA Internal Standard | 1145 | 15 (known) |
| Preparation - 5 μg/ml | | |
| (Sense Strand) | | |
| VEGF A RNA Internal Standard | 773 | 3.0 (known) |
| Preparation - 1 μg/ml | | |
| (Sense Strand) | | |
| VEGF A RNA Internal Standard | 489 | 1.5 (known) |
| Preparation - 0.5 μg/ml | | |
| (Sense Strand) | | |
| VEGF A RNA Internal Standard | 50 | 15 (known) |
| Preparation - 5 μg/ml | | |
| (Anti-sense Strand) | | |
| VEGF A RNA Internal Standard | 15 | 3.0 (known) |
| Preparation - 1 μg/ml | | |
| (Anti-sense Strand) | | |
| VEGF A RNA Internal Standard | 16 | 1.5 (known) |
| Preparation - 0.5 μg/ml | | |
| (Anti-sense Strand) | | |
| SAMPLE 1: Normal Colon | 2 | <0.01 (correlated¹) |
| SAMPLE 2: Metastatic Colon | 222 | 0.7 (correlated¹) |
| Adenocarcinoma | | |
| SAMPLE 3: Metastatic Colon | 449 | 1.4 correlated¹) |
| Adenocarcinoma | | |

*Data are expressed as Phosphorimager counts per pixel (50 micron diameter), correlated in background signal (23 units/pxel).
¹Correlated values based on Sense RNA Internal Standard Preparation data.

The data in Table 1 show that the VEGF sense RNA standards, when hybridized with an anti-sense RNA probe, result in a phosphorimager signal that increases with increasing amounts of sense RNA. As expected, the VEGF anti-sense RNA standards, when hybridized with an anti-sense RNA probe, result in a phosphorimager signal that is near background.

### Example 4

### Ouantitative Immunofluorescence Detection Using Internal Standard Preparations in a Tissue Microarray

The present example demonstrates the utility of the invention to serve as internal standards and controls in a tissue microarray (TMA) histological section for immunofluorescence (IF) procedures. In this example, an array of the invention was used to evaluate Her2/ErbB2 expression by IHC in breast tumors. Further, the present example demonstrates the utility of the invention for quantitating biologically useful molecules, such as proteins, in an array using a multiple different internal standards, each having a different quantity of the biological molecule to set up a standard curve of expression signals.

A typical tissue array containing clinical breast cancer samples and internal standard preparations was constructed containing 360 cores arrayed in 24 columns and 15 rows as follows. Three hundred twenty-six sample tissue cores measuring 0.6 mm in diameter were obtained from various donor paraffin blocks. The donor blocks included 99 specimens of mammary ductal adenocarcinoma tissue, usually sampled in duplicate or triplicate, and 2 specimens of normal mammary tissue (Leeds General Infirmary, Yorkshire, England). The sample cores were embedded into a recipient paraffin block, for example, using a Beecher tissue arraying instrument, as described in Example 3.

Eight cores of internal standard preparations measuring 0.6 mm in diameter were obtained from donor blocks containing protein/agarose, each prepared as described in Example 2 above. The protein/agarose internal standard preparations contained Her2/ErbB2 extracellular domain (ECD) protein at concentrations of 0.0046, 0.046, 0.46 and 0.93 mg/ml, each including 1% BSA, each arrayed in duplicate. The internal standard preparation cores were embedded in the paraffin recipient block in the same manner as the sample tissue cores.

Sixteen cores of cell pellet controls (A673 (ATCC catalog number CRL-1598); Calu-6 (ATCC catalog number HTB-56); NCI-H460 (ATCC catalog number HTB-177); MDA-MB-453 (ATCC catalog number HTB-131); MCF7 (ATCC catalog number HTB-22); MDA-MB-175 VII (ATCC catalog number HTB-25); MDA-MB-231 (ATCC catalog number HTB-26); NCI-H322 (ATCC catalog number CRL-5806); SK-BR-3 (ATCC catalog number HTB-30); A549 (ATCC catalog number CCL-185); and SK-MES-1 (ATCC catalog number HTB-58), which cell lines express varying levels of Her2/ErbB2) measuring 0.6 mm in diameter were embedded in the recipient paraffin block using the general methods described above. The cells for cell pellets were cultured under standard tissue culture conditions and were grown to 60-80% confluency. About 10⁷ to 10⁸ cells were collected from tissue culture plates using 7 mM EDTA in PBS (phosphate buffered saline) and incubated at 37° C. until the cells detached. The cells were then pelleted at about 300 g for 5 minutes at 4° C. in a 50 ml conical polypropylene centrifuge tube before overnight fixation in 10% NBF. Following fixation, the fixative was replaced with 70% ethanol and the sample was immediately processed for paraffin-embedding as described in Example 3. Four cores of 8% Nusieve 3:1 agarose containing 25% blue, yellow, or black surgical marking dye (useful for orientation during evaluation of the section) measuring 0.6 mm in diameter were embedded in the recipient paraffin block.

All of the cores were annealed in the recipient paraffin block array by incubating the block in a 37° C. oven overnight. For analysis, the paraffin array was sliced into one or more 3-5 μm thick histological TMA sections. Each TMA section was then transferred into a 42° C. water bath, collected individually onto Superfrost glass slides, and thoroughly dried.

Immunofluorescence (IF) was performed on some of the TMA slides from the breast cancer array using two antibodies: 1) a goat polyclonal antibody specific to the Her2/ErbB2 extracellular domain (ECD) of the naturally occurring Her2/ErbB2 receptor and a synthetic ECD sequence; and 2) a DAKO rabbit polyclonal antibody that recognizes the intracellular domain of the Her2/ErbB2 protein, which is present in naturally occurring forms of the protein but is absent in the synthetic ECD sequence. The TMA slides were deparaffinized by washing three times for 5 minutes each in Xylenes (Richard Allen Scientific, Kalamazoo, Mich.) and hydrated through graded ethanols to distilled water then rinsed twice in distilled water for 5 minutes each. The TMA slides were placed in preheated Biogenex Citra Solution, (Biogenex, San Ramon, Calif.) diluted 1:10 from a 10× stock, in distilled water for 20 minutes at 99° C. in a microwave, then cooled to room temperature for 20 minutes, and then rinsed with distilled water twice for 5 minutes each. The endogenous tissue biotin was blocked with an Avidin-Biotin blocking reagent kit following the manufacturer's recommendations (Catalog #SP-2001) (Vector Laboratories, Burlingame, Calif.). Briefly, the slides were rinsed for 10 minutes with the Avidin reagent, 10 minutes with the Biotin reagent, and then 5 minutes with PBS. The TMA slides were blocked with 10% normal horse serum in 3% BSA/PBS for 30 minutes; the blocking serum was drained from the slides. The TMA slides were then incubated with 5 μg/ml polyclonal goat antibody (human Her2/ErbB2 ECD) for 60 minutes at room temperature. On a parallel slide, 5 μg/ml of goat isotype control antibody (Neomarkers, Freemont, Calif.) was used as a negative control. After incubation, the TMA slides were rinsed with PBS twice for 5 minutes each. Next, the TMA slides were incubated with biotinylated rabbit anti-goat antibody diluted to 1:200 (final concentration was 7.5 μg/ml) in 10% normal rabbit serum, with 1% BSA in PBS for 30 minutes at room temperature. After incubation, the TMA slides were rinsed with PBS twice for 5 minutes each. Next, the TMA slides were incubated with 5 μg/ml Streptavidin conjugated to AlexaFluor 633 (Molecular Probes, Eugene, Oreg.) for 30 minutes at room temperature. After incubation, the TMA slides were rinsed with PBS twice for 5 minutes each.

To detect the DAKO rabbit anti-(human Her2/ErbB2) antibody, the same procedure was used except that the primary antibody was detected with a biotinylated goat anti-rabbit secondary antibody diluted in 10% rabbit serum, 1% BSA in PBS.

In order to view the results of the IHC procedure, the TMA slides were covered with 0.45 micrometer pore-size nitrocellulose filter paper soaked in PBS, and scanned with a Typhoon 8600 Variable Mode Imager (Molecular Dynamics). The Alexa 633 fluorescence dye was excited using a 633 nm laser and detected at a resolution of 50 μm with a 670 band-pass 30 emission filter. The image was quantified using Phoretix Array² software (Nonlinear USA Inc, Durham, N.C.) and data was analyzed using Microsoft Excel.

The amount of Her2/ErbB2 ECD protein in the internal standard preparations and the samples was calculated. The Her2/ErbB2 ECD protein in the protein/agarose internal standard preparation included approximately 650 amino acids from the N-terminus of the protein. This ECD fragment weighed about 71,400 daltons. The protein/agarose internal standard preparation contained 0.46 mg/ml of Her2/ErbB2 ECD and therefore contained about 6.44×10⁻⁹ moles/ml, or about 3.88×10¹⁵ molecules /ml, or about 3.88×10³ molecules per μm³ of Her2/ErbB2 ECD. For a histological section 5 μm thick, this standard contained approximately 1.94×10⁴ molecules of Her2/ErbB2 per square micron.

The intensity of the immunofluorescence signals resulting from the Her2/ErbB2 ECD internal standard preparation and the tissue samples were then correlated, so that the amount of Her2/ErbB2 protein in the sample could be determined in molecules of protein per unit volume of tissue. The following table summarizes the data obtained from the above-described immunofluorescence procedure.

TABLE 2

| | | QUANTITTY OF |
|---|---|---|
| | PHOSPHORIMAGER | Her2/ErbB2 |
| CONTENT OF | SIGNAL | PROTEIN¹ |
| CORE/TMA SPOT | (Goat Anti-ECD Ab)* | (molecules/μm³) |
| Her2/ErbB2 Protein Internal | 64 | 3.9 × 10³ (known) |
| Standard Preparation - 0.46 | | |
| mg/ml | | |
| Her2/ErbB2 Protein Internal | 10 | 7.8 × 10² (known) |
| Standard Preparation - 0.093 | | |
| mg/ml | | |
| Her2/ErbB2 Protein Internal | 0.8 | 3.9 × 10² (known) |
| Standard Preparation - 0.046 | | |
| mg/ml | | |
| Her2/ErbB2 Protein Internal | 1.4 | 39 (known) |
| Standard Preparation - 4.6 | | |
| μg/ml | | |
| SAMPLE 1: SkBr3 Cell Line | 290 | 1.7 × 10⁴ |
| cell pellet | | (correlated²) |
| SAMPLE 2: Breast, Ductal | 376 | 2.2 × 10⁴ |
| Adenocarcinoma | | (correlated²) |
| SAMPLE 3: Breast, Normal | 0.26 | 1.8 × 10² |
| | | (correlated²) |

*Data are expressed as Phosphorimager counts per pixel (50 micron diameter), corrected for background signal from 1% BSA cores (0.66 counts per pixel) in the same TMA. Each value represents the mean of duplicate core samples.
¹Her2/ErbB2 ECD protein standards were used to generate a 4-point standard curve relating the experimentally measured phosphorimager values per 50 μm-diameter pixel (column 2) to the known Her2 protein copy number per cubic micron (column 3). The relationship is described by the equation H = (P + 2.811)/0.0171, where H equals the HER2 copy number per cubic micron and P equals the phosphorimager value per 50 μm-diameter pixel (corrected for the BSA-only background).
²Correlated values based on Her2/ErbB2 Protein Internal Standard Preparation data. Measured data in column 2, and the relationship described in footnote 1 were used to calculate the values in column 3.

As shown above, the amount of HER2/ErbB2 receptor protein contained within the samples was quantified on the basis of the known amount of receptor protein in the internal standard preparations. The data in Table 2, above, illustrates that synthetic HER2/ErbB2 ECD standards can be used, after appropriate immunofluorescence staining, to construct a standard curve that correlates the number of HER2/ErbB2 protein molecules per unit volume of standard to phosphorimager signal strength. The data further illustrates that the standard curve can be used to correlate the measured phosphorimager signal strength for clinical tissue samples with molecules of HER2 per unit volume of tissue.

Comparison of the Quantitative Immunofluorescence Method of the Invention to Other Methods of Determining HER2 Expression or HER2 production in Cells or Tissue: The HER2/neu (c-erb-B2) oncogene, is amplified and the HER2 receptor protein is overexpressed in 20-30% of breast cancers. Women with HER2 receptor positive metastatic breast cancer have more aggressive disease, greater likelihood of recurrence, poorer prognosis, and approximately half the life expectancy of women with HER2 negative breast cancer. In current routine clinical practice, HER2 positive tumors are identified by one of two means: fluorescent in situ hybridization (FISH), which quantifies HER2 gene amplification, or by the HercepTest™, involving immunohistochemistry in which an immunoperoxidase detection method allows for semi-quantitative diagnostic scoring of tumors for HER2 receptor protein overexpression. Briefly, scoring is a method by which a pathologist assigns an integer score to an immunohistochemically stained slide based on a rough estimate of certain criteria. 3+=Complete membranous staining in >10% of cells of strong intensity. 2+=Complete membranous staining in >10% of cells with weak to moderate intensity. 1+=Incomplete membranous staining in >10% of cells. 0=Lack of staining or membranous staining in <10% of cells.

The quantitative immunofluorescence method of the invention has the advantage of high-throughput rapid quantitative detection, specificity, and improved false positive and false negative detection rate compared to commonly used methods. These advantages are illustrated by the following experiments in which cellular HER2 expression in cells of paraffin embedded tissue microarrays was determined using the quantitative immunofluorescence and quantitative immunohistochemistry methods of the invention. The results are compared to fluorescence in situ hybridization (FISH), Taqman® RT-PCR, HercepTest™ immunohistohemistry, ELISA analyses.

Cell Lines: HER2 expression was measured in nine freshly prepared cell lines by RT-PCR (Real Time Polymerase Chain Reaction), western blotting, FACS (Fluorescence-activated Cell Sorting), and ELISA (Enzyme-Linked Immunosorbent Assay). The cell lines included SK-BR-3, MDA-MB0453, NCI-H322, MDA-MB-175, MCF7, A673, A549, MDA-MB-231, SK-MES-1 (as described above). Each cell line was ranked according to the level of HER2 expression. Cell pellets from each cell line were fixed 10% neutral buffered formalin (Richard Allen Scientific, Kalamazoo, Mich.) overnight at room temperature. The cell pellet was then transferred to 70% ethanol (in water) and processed (according to standard techniques) for paraffin embedding.

Agarose HER2 ECD Standards: Recombinant HER2 receptor extracellular domain (ECD) protein was added to 2% melted agarose in dilutions of 0.46 mg/ml, 0.093 mg/ml, 0.046 mg/ml, and 0.0046 mg/ml. Each agarose block was allowed to solidify before being fixed in 10% neutral buffered formalin (NBF) and processed for embedding in paraffin and arrayed in paraffin blocks as described above.

Tissue microarrays: Representative tissue cores, in triplicate, from ninety nine paraffin-embedded grade 3 ductal breast cancers were arrayed into a single tissue microarray using a tissue arrayer (Beecher Instruments, Silver Springs, Md.) as described, for example, by Konenen et al., Nature Medicine 4(7);767-768 (1998). Also arrayed on each tissue microarray were duplicate cores of the HER2 ECD agarose standards and nine HER2-expressing cell lines. For convenience, the cell lines and agarose standards were situated in the arrays according to the known amount of HER2 receptor ECD protein in the standards and the expected amount of HER2 receptor expressed in the cell lines.

Quantitative Immunofluorescence Using HER2 ECD Standards: HER2 ECD standards embedded in agarose were prepared as follows. Melted agarose was added to a final concentration of 2% and the solution was poured into 15 mm×15 mm diSPo® histology embedding molds (Baxter Healthcare Corporation, McGaw Park, Ill.). Recombinant HER2 receptor extracellular domain (HER2 ECD) was added to the agarose and allowed to solidify. HER2 ECD was diluted to concentrations of 0.46, 0.046, and 0.0046 mg/ml. Agarose blocks were then fixed in 10% neutral buffered formalin (NBF), processed for embedding in paraffin and were arrayed in paraffin blocks.

Four antibodies were used to detect the level of HER2 protein levels in tissue, cells and standards of tissue microarrays prepared according to the procedure described in this Example 4. Any standard immunostaining procedure involving target-specific antibodies may be used. For the purposes of this example, immunostaining was performed using four different antibodies directed against the HER2 receptor protein. Two recognize the intra-cytoplasmic domain; (1) rabbit anti-human c-erbB2 (HER2/neu) polyclonal antibody (from the HercepTest™, kit, DAKO, Carpinteria, Calif.); (2) CB11 (mouse anti-human HER2 ICD monoclonal antibody (NeoMarkers, Fremont, Calif.). Two were directed against the HER2 extra-cellular domain: (3) 4D5 mouse anti-human HER2 ECD monoclonal antibody (Genentech, Inc. South San Francisco, Calif.; ATCC CRL 10463 deposited May 24, 1990; wherein rhuMAb 4D5 is Herceptin®); (4) goat anti-human HER2 ECD polyclonal antibody (Genentech, Inc. South San Francisco, Calif.). Immunostainings using rabbit anti-human c-erbB2 (HER2/neu) polyclonal antibody (HercepTest™, DAKO, supra) and CB11 antibody (NeoMarkers, supra) were performed according to the respective manufacturer's instructions. Tissues incubated with the 4D5 antibody were treated with 0.4% pepsin in 0.1N HCl for 5 minutes at 37° C. followed by an overnight incubation with 4D5 at 4° C. Tissues incubated with the goat anti-HER2 ECD antibody were pretreated with DAKO Target Retrieval Solution for 20′ at 99° C. (DAKO, Carpinteria, Calif.) followed by antibody incubation for 1 hour at room temperature. All tissues were treated with 7.5 ug/ml of species-specific biotinylated secondary antibodies (Vector Laboratories, Inc. Burlingame, Calif.). Following biotin secondary antibody binding, tissues were incubated with 5 ug/ml Streptavidin AlexaFluor 633 (Molecular Probes, Eugene, Oreg.) for 30 minutes at room temperature.

To detect fluorescence emitted by standards and samples on a microarray, any standard fluorescence detection method may be used. For the purposes of this Example, tissue samples were mounted using Vectashield Mounting Medium With DAPI (4′-6-Diamidino-2-phenylindole) (Vector Laboratories, Inc, Burlingame, Calif.). Quantitative immunofluorescence detection was performed using the Typhoon 8600 Phosphorimager (Amersham Pharmacia Biotech, Piscataway, N.J.). Samples were excited using a 633 nm laser at 350 volts and were detected with a 670 band-pass 30 emission filter at a resolution of 50 μm. Data was analyzed using Phoretix software (Nonlinear USA Inc, Durham, N.C.).

Fluorescence In situ Hybridization (FISH): FISH analysis can be performed by standard techniques. For the purposes of these experiments, the Vysis PathVysion HER2 DNA Probe Kit (Vysis, Downers Grove, Ill.) was used to determine the level of HER2 gene amplification in array tissue or cell lines. The assay and the determination of HER2 overexpression (scoring) were conducted according to the manufacturer's instructions (Vysis, supra). However, due to the small size of the tissue core sections (spots or elements) on the tissue microarrays, only ten nuclei per tissue element were examined for enumeration of each fluorescent dot corresponding to a gene locus. Using the Vysis protocol, detection of the gene, CEP17, present in alpha satellite DNA located at the centromere of chromosome 17 (17p 11.1-q11.1), relative to the copy number of chromosome 17 allowed the copy number of the HER-2 gene to be determined. The numbers of distinct HER2 and CEP17 signals were determined independently using DAPI/9-orange and DAPI/Green (Nikon) dual band-pass filters, respectively. Epi-illumination was provided by an Olympus AH2-RFL-T mercury lamp and signals were enumerated with a 100× oil-immersion objective. For each of the ten nuclei examined per element, HER2 and CEP17 signals were counted and the level of amplification was expressed as a ratio of HER2:CEP17 signals.

Tagman® RT-PCR analysis of gene expression: RNA from nine cell lines expressing HER2 was isolated with a Qiagen RNeasy midi kit (Qiagen, Inc., Valencia, Calif.). Each sample was diluted in an 11-point standard curve ranging from 50 pg to 0.05 pg of total RNA. HER2 specific primers were generated corresponding to forward 5′-TGGTCTTTGGGATCCTCATCA-3′ (SEQ ID NO:2) and reverse 5′-AGCAGTCTCCGCATCGTGTA-3′ (SEQ ID NO:3) sequences. The fluorogenic probe sequence was 5′-FAM-TCCGGATCTGCTGCCGTC-TAMRA-3′ (SEQ ID NO:4). 5-FAM™ refers to 6-carboxyfluorescein, and TAMRA™ refers to 6-carboxytetramethylrhodamine. Real time polymerase chain reaction (Taqman® RT-PCR (Applied Biosystems, Foster City, Calif.) analysis was performed according to the manufacturer's instructions. Gene expression quantitation was determined using the SYBER® green RT-PCR reagent kit (Applied Biosystems, supra).

HercepTest™: The HercepTest™ immunohistochemistry procedure was followed according to the manufacturer's guidelines (DAKO, Carpinteria, Calif.).

Goat anti-HER2 ECD immunohistochemistry: As a possible alternative to HercepTest IHC, an IHC method that used a goat anti-HER2 ECD antibody for detection was evaluated. Detection of the goat anti-HER2 ECD antibody was performed using Vectastain elite ABC-HRP kit (Vector Laboratories, Burlingame, Calif.) followed by treatment with metal-enhanced DAB (3,3′diaminobenzidine (DAB) for 10-15 minutes according to manufacturer's instructions (ImmunoPure® DAB, product no. 34001, Pierce Chemical Company, Rockford, Ill.). Fluorescence imaging for IHC was performed using, for example, the Nikon Microphot—FX scope (Nikon, Tokyo, Japan) equipped with DAPI and Rhodamine filter sets (Chroma, Brattleboro, Vt.). Images were acquired using the RT slider SPOT camera (Diagnostic instruments, Inc., Sterling Heights, Mich.). Scoring breast tumor samples as positive or negative for HER2 overexpression was determined by guidelines established by the DAKO HercepTest™ kit (DAKO, supra).

ELISA analysis: Goat anti-HER2 ECD affinity purified antibodies were diluted 1:2000 in coating buffer (0.05 M Carbonate/bicarbonate, pH 9.6). A 100 μl aliquot of antibody solution was added to a 96 well plate and incubated overnight at 4° C. Antibody solution was discarded and150 μl of blocking buffer (PBS+0.5% BSA+10 ppm Proclin) was added for 1 hr at room temperature with gentle agitation followed by 3 washes with PBS+0.05% Tween 20. An intermediate was diluted 2-fold in Magic buffer (PBS+0.5% BSA 10 ppm Proclin+0.05% Tween 20)+0.2% BgG+0.25% CHAPS+0.35M NaCl, pH 7.4) to create a 7 point standard curve ranging from 4 to 0.06 ng/ml. A 100 μl aliquot of the standard, sample and control was added to the plate in duplicate, incubated at room temperature for 2 hours with gentle agitation and washed 3×. Streptavidin-HRP (Amersham Pharmacia Biotech) was diluted 1:10,000 in MAGIC buffer annd incubated for 30 minutes at room temperature with gentle agitation then washed 3×. TMB Peroxidase Substrate was mixed with Peroxidase Solution B (H₂O₂),100 ul was added to each well, and color was allowed to develop for 10-15 minutes. Reaction was stopped with 100 μl of 1 M phosphoric acid and absorbance was read at 450-630 nm.

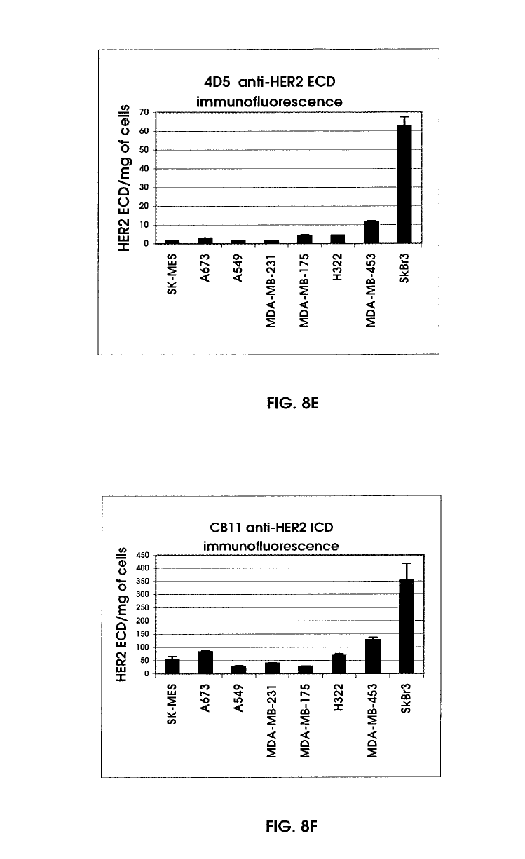

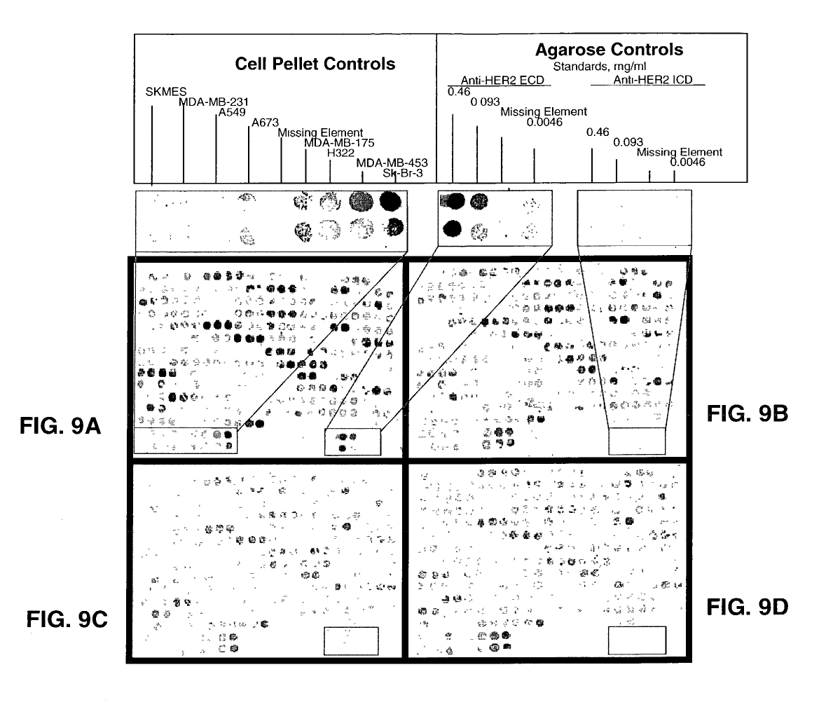

The relative amounts of HER2 gene amplification and protein expression in HER2-expressing cells on microarrays is shown by the bar graphs of FIGS. 8A-8F. The bar graphs show that the detection of HER2 in cell lines on microarrays is similar for Taqman (FIG. 8A), ELISA (FIG. 8B) and quantitative immunofluorescence using the method of the invention (FIGS. 8C-8D). This was further demonstrated by the high correlation coefficients (R²) between IF and ELISA. Quantitative immunofluorescence provided stronger signals with improved detection range, on average, relative to quantitative immunohistochemistry. In addition, the specificity of the anti-intracellular domain (anti-ICD) antibody is demonstrated by the absence of detection signal for the HER2 extracellular domain (ECD) protein standards in FIG. 9B.

Statistical analysis: The purpose of this analysis was to compare the diagnostic agreement between the results of the FISH procedure and each of the six alternate assays: HecepTest™ IHC, goat anti-HER2 ECD IHC, and quantitative immunofluorescence on microarrays comprising HER2 ECD protein embedded in agarose and detected with the four antibodies described above (rabbit anti-human c-erbB2 (HER2/neu) polyclonal antibody; CB11 (mouse anti-human HER2 ECD monoclonal antibody; 4D5 mouse anti-human HER2 ECD monoclonal antibody; and goat anti-human HER2 ECD polyclonal antibody) as applied to 98 clinical breast tissue samples. Although HecepTest™ is a currently approved method for determining eligibility for treatment with Herceptin® anti-HER2 antibody (a score of 2+ or 3+ indicates eligibility), many clinicians regard FISH analysis as more reliable than HecepTest™ for determining HER2 expression status. In addition, IHC methods require visualization of a fluorescence image by a clinician, thereby introducing possible error, reduced throughput, and high cost. Accordingly, it is useful to find another test that compares favorably with the diagnostic ability of FISH, but with higher throughput and lower cost.

FISH analysis results were used as a standard, where a FISH score greater than or equal to 2.0 was defined to be a positive HER2 overexpression score. Of the 98 breast tumor samples from 98 patients, no maximum FISH score was obtained for 10 tissue samples due to missing elements (spots) on the microarrays. Of the 88 remaining samples, 38 were classified as positive or HER2 overexpression, while 50 were negative. Using these FISH results as a standard, the performance of each of the other methods was then characterized by investigating the diagnostic scoring agreement for chosen score threshold values for the other methods. For each method, sensitivity (percentage of FISH-scored positive samples that were correctly identified as positive by the given method at its chosen threshold score value) and specificity (percentage of FISH-scored negative samples that were correctly identified as negative by the given method at its chosen threshold score value) were determined. Quantitative immunofluorescence values were normalized to results from the control MB-MDA-453 cell line present on each tissue microarray. All normalized fluorescent values ≧1 were considered positive for HER2 over-expression, equivalent to a 3+ score. The normalized immunofluorescence results were compared to those of HercepTest™ IHC assay to evaluate the relative ability of each method to correctly score samples for clinically relevant HER2 overexpression.

Currently, the scoring standard for positive HER2 overexpression by HercepTest™ is a threshold of 2+, which for these data produced sensitivity and specificity estimates of 76.3% and 92%, respectively. The results of the other assays were examined to determine the scoring thresholds producing similar sensitivity and specificity levels as given by the HercepTest™ criterion. For example, the highest 4D5 ECD threshold yielding a sensitivity of at least 76.3% was 4 IF, which corresponded to exactly 76.3% sensitivity but only 86% specificity, lower than that for the HercepTest™ assay. The lowest 4D5 ECD threshold exhibiting at least 92% specificity was 5 IF, which resulted in 94% specificity and 73.7% sensitivity. No single threshold value for the 4D5 ECD assay gives equivalent or better estimates of both sensitivity and specificity. Employing any of the other three assays (Goat IHC, Rabbit anti-HER2 ICD immunofluorescence, and 4D5 ECD immunofluorescence) resulted in reductions in either sensitivity or specificity. On the other hand, useful threshold value choices do exist for the goat anti-human HER2 ECD immunofluorescence method (61-85 IF) and the CB11 anti-human HER2 ICD immunofluorescence assay (56-59 IF), as listed in Table 3. Thus, either of these tests compares more favorably to the results of the FISH assay than the routinely used HercepTest™ assay.

TABLE 3
Comparison of assay sensitivities and specificities to Herceptest results
(76.3% sensitivity, 92% specificity).

| | Threshold value with | Threshold value with |
|---|---|---|
| | specificity ≧92% | sensitivity ≧76.3% |
| | (sensitivity %, | (sensitivity %, |
| Assay Method | specificity %) | specificity %) |
| Goat anti-HER2 ECD | 3+ (73.7%, 98%) | 2+ (92.1%, 74%) |
| immunohistochemistry | | |
| Rabbit anti-HER2 ICD | 64 IF (71.1%, 92%) | 59 IF (76.3%, 90%) |
| immunofluorescence | | |
| Goat anti-HER2 ECD | 61 IF (78.9%, 94%) | 85 IF (76.3%, 100%) |
| immunofluorescence | | |
| 4D5 anti-HER2 ECD | 5 IF (73.7%, 94%) | 4 IF (76.3%, 92%) |
| immunofluorescence | | |
| CB11 anti-HER2 ICD | 56 IF (76.3%, 92%) | 59 IF (76.3%, 92%) |
| immunofluorescence | | |

The first column lists thresholds which exhibited at least 92% specificity, while the second column gives thresholds which resulted in at least 76.3% sensitivity.

Immunofluorescence analysis of tissue microarrays using target protein standards embedded in agarose is a rapid and quantitative process useful for high throughput tissue analysis and diagnosis. The results of quantitative immunofluorescence compares favorably with other methods routinely used for target gene amplification or target protein expression, making quantitative immunofluorescence useful alone or as an adjunct to existing methods.

Comparison of the Quantitative Immunofluorescence Method of the Invention to Other Methods of Determining p53, Ki67,CD31, hMLH1 and hMSH2 Expression in Colorectal Tissue

The comparability of quantitative immunofluorescence is further demonstrated by the following experiments.

The accuracy and reliability of in situ studies are compromised by qualitative interpretations. Quantitation imposes a greater degree of objectivity and reproducibility. These experiments demonstrate the usefulness of preparing tissue microarrays with internal standards. A laser imaging system was used for the in situ quantitative analysis of gene expression in the tissue microarrays. Immunofluorescence was employed to quantify the expression of p53, Ki67,CD31, hMLH1 and hMSH2 in an arrayed series of colorectal tissues (n=110). Quantitative data on this panel of antigens were compared objectively with qualitative scoring of immunohistochemical chromogen deposition. In addition, the expression of vascular endothelial growth factor (VEGF)-A, placental growth factor, hepatocyte growth factor and c-Met mRNA were quantified by phosphor image analysis of in situ hybridization reactions. The quantified data on p53, Ki67 and CD31 expression were significantly associated with the immunohistochemical score (p≦0.001).

Microarray technology benefits from the fact that all specimens are processed under identical conditions, optimizing pre-analytical and analytical standardization. Agarose was employed as a medium to incorporate known amounts of mRNA and protein into synthetic blocks, which could be biopsied and built into the tissue microarray as internal controls. Thus, an objective of the study was to determine the utility of agarose matrices in controlling for the specificity and sensitivity of immunolabeling and ISH.

Selection of Human Tissues

FFPE colorectal tissue cassettes and corresponding hematoxylin and eosin (H&E) stained sections were reviewed for blocks containing non-neoplastic, benign, and malignant epithelial cells for microarray construction.

Preparation of Synthetic Standard Blocks

PCR primers were designed to amplify fragments of β-actin, HGF, PIGF, c-Met and VEGF-A. Sense and anti-sense HGF, PlGF, c-Met and VEGF-A RNA fragments were transcribed with the appropriate Megascript kit (Ambion, Austin, Tex.), according to the manufacturer's protocols. RNA clean-up was undertaken using the RNeasy mini kit (Qiagen Inc., Valencia, Calif.), following the manufacturer's instructions. Absorption at 260 and 280 nm was measured by spectrophotometry to determine the RNA yield and concentration.

NuSieve 3:1 agarose (FMC Bioproducts, Rockland, Md.) was made up into 250 μl aliquots of an 8% aqueous solution, and incubated in a water bath at 95° C. Serial dilutions of mRNA were prepared and mixed with the agarose by pipetting, to give final concentrations of 5 μl, 1 μg/ml and 0.5 μg/ml in a total volume of1 ml (2% agarose). The admixture was then incubated for a further 10 min at 95° C. to denature the mRNA. After thorough mixing by vortex, each control was pipetted into a 15×15 mm diSPO base mold (Baxter, Deersfield, Ill.) and allowed to set at 4° C. for 1-2 h. The solidified block was removed from the mold and fixed in 4% formalin overnight, prior to embedding in paraffin. Standards were also constructed using a peptide corresponding to the HER2 protein extracellular domain (ECD) in a similar fashion. Serial dilutions of the protein were prepared to give final concentrations of 0.46 mg/ml, 0.093 mg/ml and 0.046 mg/ml in 1 ml of 2% agarose. To prevent denaturation of the protein, the agarose was allowed to cool prior to mixing and pipetting into the mold. Blank FFPE 2% agarose controls were also incorporated into the arrays.

Tissue Microarray Construction

Two TMAs were constructed to represent the case series using a Beecher Instruments microarrayer (Silver Spring, Md.). In total, 83 primary colorectal adenocarcinomas (CRCs), 12 metastases (CRMs; 9 liver, 2 lymph nodes, 1 small intestine serosa), 15 adenomas (CRAs) and 9 adjacent normal mucosal tissues were sampled. The standards was incorporated into each microarray to become internal standards according to the invention as follows. Cylindrical cores (600 μm in diameter) were punch-biopsied from representative regions of the donor blocks and brought into recipient paraffin blocks (35×25 mm). Tissue sampling was undertaken in triplicate to provide representative data on the parent block and synthetic standards were sampled in duplicate and inserted into the recipient block. Sections, 3 μm thick, were cut from the recipient blocks and mounted on glass slides.

Immunohistochemistry

Immunolocalization of CD31, Ki67, human Mut L homologue 1 (hMLH1), human Mut S homologue 2 (hMSH2) and p53 were assessed by immunohistochemistry (IHC). Microarray sections were deparaffinized and heat-mediated antigen retrieval was performed by microwaving the slides under conditions cited in Table 1. Endogenous peroxide was quenched over 4 min at room temperature, with Kirkegaard and Perry Laboratories Blocking Solution (Gaithersburg, Md.), diluted 1:10 in deionized water. Phosphate-buffered saline (pH 7.2) was used throughout as a wash solution. Subsequently, slides were laid flat in a humidity chamber and endogenous biotin was blocked using an avidin-biotin blocking kit (Vector Labs., Burlingame, Calif.) according to the manufacturer's instructions. Non-specific immunoglobulin binding was blocked with 10% normal horse serum (NHS) (Gibco, Rockville, Md.) for 30 min at room temperature. The TMA sections were then incubated with the appropriate primary antibody diluted in NHS, under conditions cited in Table 1. Overnight incubations (CD31) were performed at 4° C. in Shandon Sequenza units (Runcorn, UK). Thereafter, the slides were incubated with the appropriate biotinylated secondary antibody (Vector Labs.), diluted 1:200 in NHS. Signal from the biotinylated antibody was amplified and labeled with horseradish peroxidase (HRP) using a streptavidin-biotin complex (ABC) (Vectastain Elite; Vector Labs.) following the supplied protocol. For CD31 immunostaining, tyramide signal amplification of the HRP complex was carried out by incubation with biotinylated tyramide (NEN TSA kit, Perkin Elmer, Boston, Mass.), followed by a second ABC layer, according to the manufacturer's protocols. Immuno-complexes were visualized by incubation with metal-enhanced 3,3′-diaminobenzidine (Pierce Technology, New York, N.Y.) for 5 min at room temperature. Tissues were counterstained with Mayer's hematoxylin, developed in bluing solution, dehydrated and mounted in synthetic media.

TABLE 4
Primary antibodies and antigen retrieval conditons employed in immunostaining.

| | Primary Antibody | | | | | Antigen Retrieval | |
|---|---|---|---|---|---|---|---|
| | | | Species | Conc. | Time and | | Time and |
| Antigen | Manufacturer | Clone | Isotype | (μg/ml) | Temp. | Buffer | Temp. |
| CD31 | DAKO Corp. | JC/70A | Mouse IgG₁ | 13.2 | 16 h 4° C. | Target | 20' 99° C. |
| ERA | DAKO Corp. | MOC-31 | Mouse IgG₁ | 1.3 | 60' 20° C. | Target | 40' 99° C. |
| HER2 | Genentech, Inc.^{a} | Polyclonal | Goat | 5.0 | 60' 20° C. | Target | 20' 99° C. |
| Ki67 | DAKO Corp. | Polyclonal | Rabbit | 2.5 | 30' 20° C. | Target | 40' 99° C. |
| hMLH1 | BD PharMingen | G168-15 | Mouse IgG₁ | 10.0 | 120' 20° C. | Trilogy | 30' 99° C. |
| hMSH2 | BD PharMingen | G219-1129 | Mouse IgG₁ | 10.0 | 120' 20° C. | Trilogy | 30' 99° C. |
| p53 | DAKO Corp. | DO-7 | Mouse IgG_{2b} | 2.5 | 60' 20° C. | Target | 20' 99° C. |

^{a}Raised against the C-terminal peptides of the HER2 protein extracellular domain.
Abbreviations:
AS, as supplied;
Conc., concentration;
ERA, epithelial-related antigen;
hMLH1/hMSH2, human Mut L/S homologue 1/2;
Temp., temperature.
BD Pharmingen, San Diego, CA; DAKO Corp., Carpinteria, CA.

Sections of FFPE human fetal block, 3 μm thick, served as experimental controls. Negative control slides were incubated with an immunoglobulin culture supernatant (DAKO Corp., Carpinteria, Calif.) of an identical species, isotype and concentration in place of the primary antibody.

All IHC was scored by a single histopathologist. Cores were assigned as overexpressing p53 if cells with positively staining nuclei accounted for greater than 25% of the epithelial cell population. The threshold for loss of expression of hMLH1 and hMSH2 was defined as complete absence of nuclear staining in the epithelial cell population of the cores. The proportion of epithelial cells demonstrating nuclear expression of Ki67 was used to assign a proliferative index, scored 1-3 (corresponding respectively to <10%, 10-50% and >50% positively stained enterocytes in each core). IHC directed against CD31 was used to determine the relative core vascularity, scored 0-3. The final score for each case was taken as the maximum from the respective three cores. To aid statistical comparison of immunofluorescent data, the Ki67 and CD31 IHC scores were reclassified into cases with a low (a score of 1) or high (a score of 2 or 3) proliferative index and a low (a score of 0 or 1) or high (a score of 2 or 3) vascular density.

Immunofluorescence

In addition to the antigens assessed by IHC, the expression of epithelial related antigen (ERA) and HER2 were also assessed by IF. Immunolabeling of the desired antigen with the primary and secondary antibodies (through to biotinylated tyramide for CD31) was performed as IHC. The biotinylated tag was then labeled with a streptavidin, Alexa Fluor 633 conjugate (diluted 1:200 in normal serum; Molecular Probes, Eugene, Oreg.) over 30 min at room temperature. Tissues were mounted and counterstained in Vectashield medium with DAPI (4′,6 diamidino-2-phenylindole) (Vector Labs.). To prevent bleaching and loss of signal, all fluorescent slides and reagents were wrapped in aluminum foil and stored at 4° C. when not in use.

Fluorescently labeled slides were first evaluated using the FLA-8000 imager (Fujifilm Medical Systems USA Inc., Stamford, Conn.) employing a 635 nm laser for excitation. A potential difference of 900 V was applied across the photo-multiplier tube for detection and quantitation of fluorescent emissions. Scanning was performed at the maximum sensitivity and achieved an image resolution of 5 μm. Subsequently, the microarrays were reviewed by fluorescent microscopy (AMJ) for verification of immunostaining.

Background subtraction, gridding and analysis of the scanned images was undertaken with Phoretix Array software (version 2; Nonlinear Dynamics, Newcastle upon Tyne, UK). The quantified signal for each case was taken as the maximum core area (CD31, ERA) or volume (all other quantified antigens) above background. The quantified IF signals (CD31, hMLH1, Ki67 and p53) were then classified by the respective binary IHC score and the interquartile ranges (IQRs) compared. Thresholds for the appraisal of quantitative IF were set between the 3rd quartile of the group with the lowest median area/volume and the 1st quartile of the group with the highest median area/volume. Each case was then assigned a binary score relative to the chosen threshold.

In situ Hybridization

[³³P]UTP-labeled (Amersham Pharmacia Biotech, Piscataway, N.J.) anti-sense riboprobes were transcribed in vitro, from the amplified β-actin, VEGF-A, HGF, c-Met and PIGF cDNA templates. TMA sections were deparaffinized, deproteinated in 4 μg/ml proteinase K for 30 min at 37° C. and further processed for ISH using standard methods (See, for example, Lu L. H. and Gillett N. A., Cell Vision 1:169-176 (1994); and Weisinger G. et al., Biochim Biophys Acta 1446:225-232 (1999). Anti-sense riboprobes were hybridized at 55° C. overnight, followed by a high stringency wash at 55° C. in 0.1×SSC for 2 hr. For quantitative analysis, dried, isotopically hybridized slides were apposed to a phosphor imaging plate (IP) (85×127 mm with exposure cassette, Fujifilm) for 18 hours at room temperature. Immediately post-incubation, the IP was scanned at a resolution of 10 μm with a phosphor imager (FLA-8000), employing a 532 nm laser for excitation and a B390 filter to detect photo-stimulated luminescence. Background subtraction, gridding and analysis of the IPs was undertaken with Phoretix Array software. The quantified signal for each case was taken as the maximum core volume above background. To control for variations in the mRNA content of the cores, the signals from probes hybridized to VEGF-A, c-Met, HGF and PlGF were normalized to the signal from the probe hybridized to β-actin (See Frantz G. D. et al., J Pathol 195:87-96 (2001).

After IP exposure, the slides were dipped in NBT2 nuclear track emulsion (Eastman Kodak, Rochester, N.Y.), exposed in sealed plastic slide boxes containing desiccant for 2-4 weeks at 4° C., developed and counterstained with H&E. Subsequently, the microarrays were reviewed by bright/dark-field microscopy for verification of hybridization.

Statistical Analysis

Statistical analysis was performed using SPSS for Windows (version 10.1; Chicago, Ill.). Pearson's χ² test with Yates' correction was used to assess the significance of associations between categorical data; where the expected counts were less than 5, Fisher's exact test was used. The mean and median values of continuous data were compared by Student's t-test and the Mann-Whitney U test respectively. Statistical significance was assumed if the two-sided p value was <0.05.

Results

Immunohistochemistry

Between 85 and 103 of 119 cases sampled (71-86%) were adequate for interpretation of antigen expression (Table 5). Elements were deemed inadequate if they contained insufficient epithelial cells, tissue necrosis or hemorrhage. All negative control sections demonstrated an absence of chromogen deposition. The anti-p53 immunoglobulin clone DO-7 is sensitive to both wild-type and mutant forms of p53 protein. Chromogen deposition was observed in the nuclei of the epithelial cell population only. Expression of p53 was evident in 40/70 CRCs compared with 0/9 of adjacent normal mucosae (p=0.001) and only 1/12 CRAs (p=0.005). In contrast, the expression of Ki67, hMLH1 and hMSH2 was observed in the nuclei of both epithelial cells and intervening stroma. In normal enterocytes, a gradual loss of expression of all three antigens was observed with progression of the cells through the crypt. Loss of one mismatch repair protein (hMLH1) was detected in 7/73 neoplasms (9.6%), whereas hMSH2 was expressed by all tumors scored (n=76). All adjacent non-neoplastic mucosal cores expressed hMLH1 and hMSH2. The Ki67-proliferation index was significantly higher in primary CRCs (60/67 scored 2-3) as compared to adjacent normal mucosa (0/9 scored 2-3; p=0.001) and CRAs (6/15 scored 2-3; p<0.001). CD31 expression was observed specifically in platelets and the plasma membrane of endothelial cells. Cores with hemorrhage (n=9) were excluded, as gross extravasation of platelets into the surrounding tissues precluded a reliable score. The CRAs sampled were significantly less vascular (2/12 scored 2-3) than either adjacent normal mucosa (6/9 scored 2-3; p=0.012) or CRCs (39/68 scored 2-3; p=0.032). The expression profile of CRMs was not significantly different from that of CRCs. (Summarized in Table 5).

TABLE 5
Immunohistochemical scores of protein expression in colorectal tissues.

| | | Normal | CRAs | CRCs | CRMs | Total |
|---|---|---|---|---|---|---|
| p53 | 0 | 9 (100%) | 11 (92%) | 30 (43%) | 8 | 58 |
| (n = 103) | | | | | (67%) | (56%) |
| | 1 | 0^{a} | 1^{b} (8%) | 40 (57%) | 4 | 45 |
| | | | | | (33%) | (44%) |
| | Total | 9 | 12 | 70 | 12 | 103 |
| hMLH1 | 0 | 0 | 1 (8%) | 5 (9%) | 1 | 7 |
| (n = 89) | | | | | (11%) | (9%) |
| | 1 | 9 (100%) | 11 (92%) | 54 (91%) | 8 | 82 |
| | | | | | (89%) | (91%) |
| | Total | 9 | 12 | 58 | 9 | 89 |
| hMSH2 | 1 | 9 (100%) | 10 (100%) | 57 (100%) | 9 | 85 |
| (n = 85) | | | | | (100%) | (100%) |
| | Total | 9 | 10 | 57 | 9 | 85 |
| Ki67 | 1 | 9 (100%) | 9 (60%) | 7 (10%) | 2 | 27 |
| (n = 102) | | | | | (18%) | (26%) |
| | 2 | 0 | 5 (33%) | 22 (33%) | 6 | 33 |
| | | | | | (55%) | (32%) |
| | 3 | 0^{c} | 1^{d} (7%) | 38 (57%) | 3 | 42 |
| | | | | | (27%) | (42%) |
| | Total | 9 | 15 | 67 | 11 | 102 |
| CD31 | 0, 1 | 3 (33%) | 10 (83%) | 29 (43%) | 5 | 47 |
| (n = 100) | | | | | (45%) | (47%) |
| | 2, 3 | 6 (67%) | 2^{e,f} (17%) | 39 (57%) | 6 | 53 |
| | | | | | (55%) | (53%) |
| | Total | 9 | 12 | 68 | 11 | 100 |

Two-sided statistical significance :
^{a}p = 0.001 (Normal vs CRCs),
^{b}p = 0.005 (CRAs vs CRCs),
^{c}p = 0.001 (Normal vs CRCs),
^{d}p < 0.001 (CRAs vs CRCs),
^{e}p = 0.012 (CRAs vs Normal),
^{f}p = 0.032 (CRAs vs CRCs).

p53 expression was not observed in tumors with loss of hMLH1 as compared to 53% of tumors with retained hMLH1 (0/7 vs. 39/74; p=0.012). No other antigens demonstrated a statistically significant association (data not shown).

Quantitative Immunofluorescence

Between 81 and 100 of 119 cases sampled (68-84%) were adequate for analysis on each section (Table 6). The exclusion of cores with an inadequate epithelial cell content was guided by both ERA IF and review of the slides with fluorescent microscopy. This was not significantly different from the number of IHC-stained cases that were valid for appraisal. Immunolocalization of the fluorescent signal, viewed by fluorescent microscopy and quantitative imaging, was identical to the pattern of chromogen deposition for each respective antibody.

ERA expression was observed in the membrane of both normal and neoplastic enterocytes. However, the intensity of fluorescence was not uniform throughout the epithelial cell population. With the exception of hMLH1, the IHC-classified IQRs formed distinct, non-overlapping groups. The quantitative IF scores of p53, Ki67 and CD31 expression were significantly associated with the qualitative scores appraised by IHC (Table 6; p≧0.001).

TABLE
A comparison of quantitative immunofluorescence with observer-scored
immunohistochemistry.

| | | p53 | | | Ki67 | | | CD31 | | |
|---|---|---|---|---|---|---|---|---|---|---|
| IHC Score | | 0 | 1 | Total | 1 | 2, 3 | Total | 0, 1 | 2, 3 | Total |
| IF | 0 | 43 | 4 | 47 | 12 | 13 | 25 | 37 | 13 | 50 |
| Score | 1 | 1 | 33 | 34 | 10 | 56 | 66 | 10 | 40 | 50 |
| Total | | 44 | 37 | 81 | 22 | 69 | 91 | 47 | 53 | 100 |

However, the distribution of the quantitative IF demonstrated wider IQRs in cases with higher qualitative IHC scores (ranging from 1.6 to 8.1 fold greater). Absolute agreement between the IHC and quantitative IF scores was close to 1 for p53 (κ=0.88), but relatively low for Ki67 (κ=0.34) and CD31 (κ=0.54) (Table 7). Likewise, the predictive value of the quantitative assay was greatest for antigens that were expressed in specific cell populations (p53, CD31), and were thus not subject to confounding signals from adjacent cell types (Table 7). In contrast, Ki67, which may be expressed in either or both stromal and epithelial cell populations, demonstrated a comparatively low negative predictive value (Table 7).

TABLE 7
Predictive value of quantitative immunofluorescence compared with
observer-scored immunohistochemistry.

| Antigen | p53 | Ki67 | CD31 |
|---|---|---|---|
| Pearson's χ² test with Yates' | p < 0.001 | p = 0.001 | p < 0.001 |
| correction | | | |
| Kappa proportional agreement | 0.88 | 0.34 | 0.54 |
| statistic | | | |
| Positive predictive value | 0.89 | 0.84 | 0.78 |
| Negative predictive value | 0.98 | 0.48 | 0.75 |

The synthetic internal standard cores evidenced a positive signal gradient in accordance with the concentration of HER2 ECD. The signal from the HER2 control cores did not exceed the background level in all other IHC, IF and ISH studies and the blank agarose control cores were negative. HER2 was expressed at low levels in the majority of colorectal epithelial tissues examined. Nonetheless, the median expression of HER2 was elevated 1.8 to 2.9 fold in neoplastic populations as compared with the normal adjacent mucosa (p≧0.008. High levels of expression, though, were observed in a single serosal metastasis, which evidenced a 4.9 fold greater signal volume than the mean signal from other malignancies on the array (70,536 vs. 14,408 relative units; p<0.001).

Quantitative ISH

The synthetic sense mRNA internal standards hybridized specifically with the appropriate anti-sense riboprobe and demonstrated a positive gradient of phosphor-luminescence with increasing mRNA concentrations. In contrast, the signal from the anti-sense and blank agarose controls did not exceed background luminescence. Bright/dark-field microscopy demonstrated expression of β-actin in all colorectal cell populations. Cases with a β-actin signal volume below the first quartile were deemed to contain insufficient mRNA for analysis and were excluded from the interpretation. β-actin was expressed at a sufficiently high level in 88 of 119 cases sampled (74%).

On review of the scanned images, quantitative data, and bright/dark-field microscopy, no colorectal tissues evidenced hybridization of the radiolabeled riboprobes directed against HGF and PlGF mRNA. In contrast, c-Met and VEGF-A were upregulated in a subgroup of CRAs, CRCs and CRMs. A proportion of CRAs and CRCs evidenced upregulation of c-Met mRNA expression up to 1.9 and 2.7 fold greater than the expression in normal mucosa respectively. Significant VEGF-A expression was observed by bright/dark-field microscopy. The median level of VEGF-A mRNA was elevated 4 fold in CRCs compared with the adjacent normal mucosa (0.20 vs. 0.05; p=0.003), although, VEGF-A expression was not significantly different between CRAs and CRCs (median, 0.14 vs. 0.20; p=0.387) or between CRCs and CRMs (median, 0.20 vs. 0.29; p=0.718). In total, 74/88 (84%) of colorectal tumors demonstrated increased expression of VEGF-A. VEGF-A expression was 2.7 fold higher in cases with expression of p53 above the threshold for immunohistochemical detection (median, 0.225 vs. 0.084; p=0.025) and 2.2 fold higher in cases with a proliferative index scored 2 or 3 (median, 0.169 vs. 0.077; p=0.014).

This study demonstrated the utility of a novel high-resolution laser imaging system for the rapid quantitation of IF and ISH. In addition, the data yielded important information on the molecular changes believed to underlie colorectal cancer progression and tumor-associated angiogenesis.

Quantitative IF using internal standards in a tissue microarray and directed against p53 and CD31 showed high levels of concordance with the IHC score (Table 7, supra). The quantitative data was distributed over a wide range within observer-defined categories that had a high IHC score. This indicates that the IHC observer cannot accurately discern differences in chromogen distribution and/or intensity when the immunolabeling is dense, and may be missing data of clinicopathological significance. Likewise, the observer cannot accurately differentiate subtle differences in expression. For example, quantitative imaging was able to discern a general increase in HER2 expression with neoplastic transformation, which was not apparent by fluorescent microscopy. This study also evidenced p53 expression late in colorectal tumorigenesis and found it to be inversely associated with loss of hMLH 1.

Qualitative appraisal of IHC, ISH and RT-PCR does not adequately appraise VEGF expression, which is a continuous variable. In contrast, the quantitative approach using internal standards on a tissue microarray described herein provides a more accurate measure of gene expression. In addition to accurate quantitation, the superior morphology of FFPE tissues allowed the unequivocal localization of labeled antigens and mRNA transcripts.

Increased VEGF Expression in Renal Cell Carcinoma Relative to Controls Correlates with Increased HIF-1α Nuclear Expression.

Oxygen availability plays a major role in the regulation of expression of many different genes including erythropoietin, nitric oxide synthase (NOS), heme oxygenase 1 (HO-1), glucose transporters and vascular growth factors (such as VEGF) necessary for the maintenance of homeostasis in hypoxic conditions. Hypoxia-inducible factor 1 alpha (HIF-1α) has been identified as a bHLH-PAS family member which is instrumental in the oxygen-dependent regulation of these genes. HIF-1α rapidly accumulates in the nucleus upon exposure to hypoxic conditions where it heterodimerizes with the aryl hydrocarbon nuclear receptor translocator, ARNT, also referred to as HIF-1 beta. The relative expressions of VEGF and HID-1α in various carcinomas was evaluated by quantitative in situ hybridization according to the procedures described herein.

Standards were prepared as described herein. mRNA encoding VEGF and HIF-1α were transcribed and embedded in agarose with BSA as described herein. Tissue microarrays comprising samples from various tissues including normal control tissues as well as carcinomas of breast, lung, colon, ovary, thyroid, kidney, and sarcomas were examined for relative quantitative expression of VEGF and HIF-1α.

Expression of VEGF and/or HID-1α was detected in multiple tumor types. VEGF expression was highest in renal cell carcinoma, but was also expressed above normal controls in lung, ovarian and thyroid carcinomas. In renal cell carcinomas having mutations in the VHL gene, VEGF expression correlated with HIF-1α expression. There was little correlation between the level of VEGF mRNA and the presence of HIF-1α mRNA in other tumor types examined. Thus, detecting increased expression of VEGF and HIF-1α above normal control tissues by quantitative in situ hybridization is a useful method for detecting renal cell carcinoma in a patient.

Taken together, the results presented herein demonstrates that laser imaging of tissue microarrays comprising internal standards is a useful method for the in situ surveillance of arrayed tumor populations. The approach meets requirements for a high-throughput, reproducible and standardized method that is applicable to FFPE tissues and offers quantitative data over a wide dynamic range. While IF-labeling and phosphor imaging plates are not amenable to long-term storage, digital imaging allows a high-resolution electronic record to be stored in a virtual archive. This would facilitate the retrospective analysis of experimental data and may form an integral part of a structured TMA database.

### Example 5

### Multiple RNA Molecules/Agrose Internal Standard Preparation

The present example demonstrates the utility of the invention for using an internal standard preparation having a known quantity of different biological molecules, such as different types of RNA, in a solid embedding material, such as agarose. Specifically, the present example demonstrates an approach for embedding a multiple different RNA molecules in agarose and BSA to form an internal standard preparation for use in an array so that the RNAs are retained throughout processing and analytical procedures performed on the array. The embedded RNA molecules can be used simply as a positive control for procedural success, particularly for procedures in which two different RNA molecules might be detected using different labels, as a component in a basic assay to improve upon procedural methods, for example for double-labeled in-situ hybridization, or ultimately as a quantitative standard to assess comparative levels of gene expression in tissues or cells.

Sense transcripts of liv-1 RNA were transcribed in-vitro using a PCR-amplified DNA template having the following sequence (sense orientation):

| | |
|---|---|
| TGCCATTCACATTTCCACGATACACTCGGCCAGTCAGACGATCTCATTCACCA | [SEQ ID NO: 5] |
| CCATCATGACTACCATCATATTCTCCATCATCACCACCACCAAAACCACCATC | |
| CTCACAGTCACAGCCAGCGCTACTCTCGGGAGGAGCTGAAAGATGCCGGCGTC | |
| GCCACTTTGGCCTGGATGGTGATAATGGGTGATGGCCTGCACAATTTCAGCGA | |
| TGGCCTAGCAATTGGTGCTGCTTTTACTGAAGGCTTATCAAGTGGTTTAAGTA | |
| CTTCTGTTGCTGTGTTCTGTCATGAGTTGCCTCATGAATTAGGTGACTTTGCT | |
| GTTCTACTAAAGGCTGACATGACCGTTAAGCAGGCTGTCCTTTATAATGCATT | |
| GTCAGCCATGCTGGCGTATCTTGGAATGGCAACAGGAATTTTCATTGGTCATT | |
| ATGCTGAAAATGTTTCTATGTGGATATTTGCACTTACTGCTGGCTTATTCATG | |
| TATGTTGCTCTGGTTGATATGGTACCTGAAATGCTGCACAATGATGCTAGTGA | |
| CCATGGATGTAGCCGCTGGGG | |

Sense transcripts of DrC3 RNA were transcribed in-vitro using a PCR-amplified DNA template having the following sequence (sense orientation):

| | |
|---|---|
| CAGCCAGAACACGCAGTGCCAGCCGTGCCCCCCAGGCACCTTCTCAGCCAGCA | [SEQ ID NO: 6] |
| GCTCCAGCTCAGAGCAGTGCCAGCCCCACCGCAACTGCACGGCCCTGGGCCTG | |
| GCCCTCAATGTGCCAGGCTCTTCCTCCCATGACACCCTGTGCACCAGCTGCAC | |
| TGGCTTCCCCCTCAGCACCAGGGTACCAGGAGCTGAGGAGTGTGAGCGTGCCG | |
| TCATCGACTTTGTGGCTTTCCAGGACATCTCCATCAAGAGGCTGCAGCGGCTG | |
| CTGCAGGCCCTCGAGGCCCCGGAGGGCTGGGGTCCGACACCAAGGGCGGGCCG | |
| CGCGGCCTTGCAGCTGAAGCTGCGTCGGCGGCTCACGGAGCTCCTGGGGGCGC | |
| AGGACGGGGCGCTGCTGGTGCGGCTGCTGCAGGCGCTGCGCGTGGCCAGGATG | |
| CCCGGGCTGGAGCGGAGCGTCCGTGAGCGCTTCCTCCCTGTGCACTGATCCTG | |
| GCCCCCTCTTATTTATTCTACATCCTTGGCACCCC | |

VEGF A transcripts were transcribed as described in Example 1.

Three different internal standard preparations were created using the methods described in Example 1: one containing only VEGF A sense RNA; a second containing liv-1 sense RNA and DcR3 sense RNA; and a third containing liv-1 sense RNA, DcR3 sense RNA, and VEGF A sense RNA. The first internal standard preparation containing VEGF A RNA was made exactly according to the procedure described in Example 1. The second internal standard preparation was made by adding liv-1 and DcR3 RNA such that the final concentration of each RNA was 100 ng/mL. (For example: 1 μl 100 ng/μl liv-1 RNA+1 μl 100 ng/μl DcR3 RNA+250 μl 8% agarose+748 μl SQH2O.) The third internal standard preparation was made by adding VEGF A, liv-1 and DcR3 RNA such that the final concentration of each RNA was 100 ng/mL. (For example: 1 μl 100 ng/μl liv-1 RNA+1 μl 100 ng/μl DcR3 RNA+1 μl 100 ng/μl VEGF A RNA+250 μl 8% agarose+747 μl SQH2O).

Each of the three internal standard preparations, contained in separate Eppendorf tubes, were heated in a 95° C. heat block for 3 minutes, and then chilled immediately on ice to denature the RNA transcripts. To each of the RNA solutions, 250 ml of 8% NuSieve 3:1 (a high gel strength agarose) and 500 ml SQH₂O that had been warmed in a 50° C. heat block were added as described in Example 1. Each of the RNA/agarose internal standard preparations were vortexed briefly and then poured into a 15 mm×15 mm DisPO base mold (Baxter Scientific). The RNA/agarose internal standard preparations were then allowed to gel at 4° C. for at least one hour to form a donor block. Each of the RNA/agarose donor blocks were fixed as described in Example 1.

A TMA was created as described in Example 3 with triplicate cores containing the three internal standard preparations (VEGF A transcripts alone, DcR3 and liv-1 transcripts, and DcR3, liv-1 and VEGF A transcripts). The TMA was hybridized with the same VEGF A anti-sense probe used in Example 3. Only the internal standard preparation cores containing VEGF A sense transcripts, either alone or in combination, gave positive signal.

TABLE 8

| | PHOSPHOR- | POSITIVE |
|---|---|---|
| CONTENT OF CORE/TMA | IMAGER | DETECTION OF |
| SPOT | SIGNAL* | VEGF A RNA |
| First Internal Standard Preparation | 2011 | YES |
| (VEGF A sense RNA alone) | | |
| Second Internal Standard | 1 | NO |
| Preparation (DcR3 and liv-1 | | |
| RNA) | | |
| Third Internal Standard | 135 | YES |
| Preparation (DcR3, liv-1, and | | |
| VEGF A sense RNA) | | |

*Data are expressed as Phosphorimager counts per pixel (50 micron diameter), corrected for background signal at the edge of each spot. Each value represents the mean of triplicate core samples.

As shown in Table 8 above, an internal standard preparation containing multiple different kinds of RNA molecules will give positive results when hybridized with a probe for an individual RNA in a mixture of RNA molecules, as illustrated here for VEGF A.

### Example 6

### RNA/Protein/Agarose Internal Standard Preparation

The present example demonstrates the utility of the invention for using an internal standard preparation having a known quantity of different biological molecules, such as an RNA molecule and a protein, in a solid embedding material, such as agarose. Specifically, an internal standard preparation prepared as described in Examples 1 and 2 containing both protein and RNA molecules could be used as a positive control for procedural success particularly for procedures in which RNA and protein expression is evaluated in the same section by immunohistochemistry and in-situ hybridization procedures, as a component in a basic assay to improve upon procedural methods, for example for detection of RNA and protein in the same section, or ultimately as a quantitative standard to assess comparative levels of RNA and protein expression in tissues or cells.

Her2/ErbB2 sense RNA transcripts are transcribed in-vitro to make an RNA solution as described in detail in Example 1. A working concentration of 100 ng/μl of the Her2/ErbB2 RNA solution is made as described in Example 1. A 50 μl aliquot of the RNA solution (5 μg) is added to 200 μl of SQH₂O in a new Eppendorf tube. The Eppendorf tube containing the RNA solution is heated in a 95° C. heat block for 3 minutes to denature the RNA transcript, and then chilled immediately on ice. Separate from the RNA solution, a final concentration of 0.45 mg/mL of Her2/ErbB2 protein is made by adding 500 μl of 0.93 mg/mL of synthetic Her2/ErbB2 ECD protein as described in Example 2. The protein/water mixture is vortexed briefly, then added to the 250 μl RNA solution. Next, 250 μl of 8% NuSieve 3:1 (a high gel strength agarose melted at 99° C.) that is cooled briefly to approximately 60° C. is added to the RNA/protein mixture. The RNA/protein/agarose mixture is vortexed briefly and then poured into a 15 mm×15 mm DisPO base mold (Baxter Scientific). The RNA/protein/agarose mixture is allowed to gel at 4° C. for at least one hour. To vary the concentration of RNA, protein, or agarose, the volume of the component can be increased with a reciprocal reduction in the amount of SQH₂O. After the gel is formed, the RNA/protein/agarose blocks can be processed as described in Examples 1-4.

### EXAMPLE 7

### Construction of a Frozen Cell Array

The present example demonstrates the utility of the invention for constructing a frozen cell array.

An arrayer was made having 25 pins, comprising hollow glass pins, i.e., glass blunts, measuring 40 mm long×1.2 mm in outer diameter, were heat-sealed and glued with Epoxy in a base made of Plexiglas measuring 12 mm×12 mm (144 mm²). Each pin was equally spaced 1.4 mm apart and plugged with a sealer comprising small pieces of metal and epoxy. A fluid OCT medium was poured into a disposable embedding mold (VWR, San Francisco, Calif.) measuring 22 mm×30 mm×20 mm (deep). The arrayer pins were first immersed in glycerol and then partially immersed in a fluid OCT medium contained within the embedding mold. The fluid OCT was frozen by submerging the fluid OCT, the mold, and the engaged pins in a cryobath of isopentane at −160° C. for 3 to 5 minutes. The pins were then extracted from the OCT mold leaving an array of 25 wells at least 20 mm deep in an array recipient block. The array recipient block was stored at −70° C. until the wells were loaded with various cell line suspensions.

Both adherent and suspension cells, listed in the following chart, were used added to the frozen array. The adherent cells were detached from tissue culture flasks in the presence of 0.5 mM EDTA for 15-20 minutes at room temperature, then centrifuged at 1000 rpm for 5 minutes, and washed in PBS at 4° C. Suspension cells (COLO205, Jurkat, and Bjab) were directly washed in PBS at 4° C. Cell number was determined by using a particle count analyzer (Coulter Z2, Beckman Coulter) and the cells were resuspended in 70 to 150 μl of cold PBS in order to obtain a highly concentrated cell suspension. The cell suspensions were maintained at 4° C. until loading. The final density of the cell suspensions that were loaded into the array are shown in Table 9.

TABLE 9

| | Concen- | | |
|---|---|---|---|
| Cell Type | tration | Origin | Source |
| BKGE | 92.9 × 10⁶ | Bovine kidney | VEC Technologies, Inc |
| | cells/ml | glomerular | |
| | | endothelial cell | |
| COLO205 | 259.3 × 10⁶ | Human colorectal | ATCC |
| | cells/ml | carcinoma cell line | Cat # CCL-222 |
| U87MG | 86.6 × 10⁶ | Human neuroglioma | ATCC |
| | cells/ml | cell line | Cat # HTB-138 |
| DU145 | 52.9 × 10⁶ | Human prostate | ATCC |
| | cells/m | carcinoma cell line | Cat # HTB-81 |
| HIAEC | 60.4 × 10⁶ | Human iliac artery | BioWhittaker/Clonetics |
| | cells/ml | endothelial cells | Cat # CC-2545 |
| HMVEC | 124 × 10⁶ | Human | BioWhittaker/Clonetics |
| | cells/ml | microvascular | Cat # CC-2543 |
| | | endothelial cells | |
| | | from lung | |
| CASMC | 45.1 × 10⁶ | Human coronary | BioWhittaker/Clonetics |
| | cells/ml | artery smooth | Cat # CC-2583 |
| | | muscle cells | |
| A375 | 79.4 × 10⁶ | Human malignant | ATCC |
| | cells/ml | melanoma cell line | Cat # CRL-1619 |
| MCF7 | 26 × 10⁶ | Human breast | ATCC |
| | cells/ml | carcinoma cell line | Cat # HTB-22 |
| A673 | 172.7 × 10⁶ | Human | ATCC |
| | cells/ml | rhabdomyosarcoma | Cat # CRL-1598 |
| | | cell line | |
| Hep3b | 57.4 × 10⁶ | Human liver | ATCC |
| | cells/ml | carcinoma cell line | Cat # HB-8064 |
| Bjab | 335.5 × 10⁶ | B cell leukemia cell | ATCC |
| | cells/ml | line | Cat # HB-136 |
| HCT116 | 82.5 × 10⁶ | Human colorectal | ATCC |
| | cells/ml | carcinoma cell line | Cat # CCL-247 |
| SW620 | 95 × 10⁶ | Human colorectal | ATCC |
| | cells/ml | carcinoma cell line | Cat # CCL-227 |
| PC3 | 56.4 × 10⁶ | Human prostate | ATCC |
| | cells/ml | carcinoma cell line | Cat # CRL-1435 |
| NRP154 | 74.1 × 10⁶ | Tumorigenic adult | Marker P. C. et al., |
| | cells/ml | rat prostate | Developmental Biology |
| | | cell line | (2001), 233, 95-108 |
| HUVEC | 65 × 10⁶ | Human umbilical | BioWhittaker/Clonetics |
| | cells/ml | vein endothelial | Cat # CC-2517 |
| | | cells | |
| NHDF | 36.1 × 10⁶ | Normal human | BioWhittaker/Clonetics |
| | cells/ml | dermal fibroblasts | Cat # CC-2509 |
| NHEK | 42.3 × 10⁶ | Neonatal normal | BioWhittaker/Clonetics |
| | cells/ml | human epidermal | Cat # CC-2507 |
| | | keratinocytes | |
| SkBr3 | 53.3 × 10⁶ | Human breast | ATCC |
| | cells/ml | carcinoma cell line | Cat # HTB-30 |
| BT474 | 72.4 × 10⁶ | Human breast | ATCC |
| | cells/ml | carcinoma cell line | Cat # HTB-20 |
| HepG2 | 66.6 × 10⁶ | Human liver | ATCC |
| | cells/ml | carcinoma cell line | Cat # HB-8065 |
| Jurkat | 614.4 × 10⁶ | T cell leukemia cell | ATCC |
| | cells/ml | line | Cat # TIB-152 |
| SKMES | 73.9 × 10⁶ | Human lung | ATCC |
| | cells/ml | carcinoma cell line | Cat # HTB-58 |

To load the cells, the OCT array was removed from cold storage and placed onto a box filled with dry ice at room temperature. The array positions were numbered A-E for the columns and 1-5 for the rows for a total of 25 positions. Position A1 of the array was loaded with Trypan blue stain 0.4% (BivcoBRL) as a orientation marker. Less than 100 μl of each of the aforementioned 24 cell suspensions were loaded into the remaining wells of the array using 1 ml syringes with 22-gauge, 1.5 inch long (0.7 mm×40 mm) needles (Becton Dickinson & Co., Bedford, Mass.). The array was stored at −70° C. until sliced for array slides.

One or more sections of 6 μl and 12 μm thickness were cut from the above array on a cryostat −20° C. and laid onto pre-cleaned microscope slides (75 mm×25 mm, 0.96 to 1.09 mm thick) (Baxter Diagnostic Inc.). Each slide contained 2 sections of the cell array (1.44 cm²) with each spot measuring 1.1 mm in diameter. The cell array slides were stored at −70° C. until used for analysis.

### Example 8

### Immunohistochemistry on a Frozen Cell Array

The present example demonstrates the utility of the invention for performing an immunochemistry procedure on a section of frozen cell array.

The frozen cell array slide of Example 7 containing multiple cell samples was air-dried at room temperature for at least 3 hours before it was fixed in acetone for 5 minutes and air-dried overnight. Endogenous immunoglobulin binding sites were blocked with PBS 1% BSA for 30 minutes and then were overlaid for 1 hour at room temperature with PBS 1% BSA containing 0.5 μg/ml of mouse anti-human Ep-CAM fluorescein-conjugated monoclonal antibody (Biomedia Corp., Foster City, Calif.). After several rinses in PBS, sections were treated with a nuclear counterstain (100 μg/ml) (Hoechst 33342, Molecular Probes, Eugene, Oreg.) for 2 minutes and rinsed again before mounting with a Vectashield mounting medium (Vecta Laboratories, Burlingham, Calif.) and a cover glass (22 mm², N°1, Corning) for viewing. The slides were stored with protection from light and dust until performing the immunochemistry procedure.

Histochemical staining of array spots resulted in heterogeneous signals from spot to spot across an array. Where the different cell types present on the array were not loaded at the same cell density, the Hoechst signal intensity appeared different on each spot. For example, the spot corresponding to Jurkat cells loaded at the highest density (614.4×10⁶ cells/ml) appeared the brightest and the spots loaded with a cell suspension of NHDF, CASMC, NHEK and MCF7 cells at a density lower than 50×10⁶ cells/ml appeared the faintest.

Some of the slides were analyzed for the presence of cell surface Ep-CAM using the mouse anti-humna Ep-CAM fluorescein-conjugated monoclonal antibody (Biomedia Corp., supra). The fluorescein signal was captured using a fluorescent microscope and a Typhoon 8600 scanner. The strongest fluorescein signal (Ep-CAM) was observed for cell lines COLO205, HCT116, SW620, HepG3 and SkBr3. Detectable fluorescein signal was also observed for cell lines DU145, NRP154, MCF7, BT474 and HepG2. A very weak signal was seen for cell lines CASMC, HUVEC and U87MG. No signal was observed in other cell lines on the cell array, including BKGE, PC3, HIAEC, HMVEC, NHDF, NHEK, A375, A673, Jurkat, Bjab and SKMES 1.

This data illustrates that the frozen cell array described herein provides reliable protein expression data for a broad protein expression screening.

### Example 9

### In-Situ Hybridization on a Frozen Cell Array

The present example demonstrates the utility of the invention for performing a in-situ hybridization on a section of frozen cell array.

The integrity of the preservation of the RNA in the frozen samples in the frozen cell array was evaluated by hybridization with RNA probes for cytoplasmic-actin according to the following protocol. The sequence of the PCR-amplified DNA template (sense orientation) used to transcribe the human B-actin RNA probe used below was:

| | |
|---|---|
| GCTGCCTGACGGCCAGGTCATCACCATTGGCAATGAGCGGTTCCGCTGCCCTGA | [SEQ ID NO: 7] |
| GGCACTCTTCCAGCCTTCCTTCCTGGGCATGGAGTCCTGTGGCATCCACGAAAC | |
| TACCTTCAACTCCATCATGAAGTGTGACTGTGACATCCGCAAAGACCTGTACGC | |
| CAACACAGTGCTGTCTGGCGGCACCACCATGTACCCTGGCATTGCCGACAGGAT | |
| GCAGAAGGAGATCACTGCCCTGGCACCCAGCACAATGAAGATCAAGATCATTGC | |
| TCCTCTGAGCGCAAGTACTC | |

Frozen slides were allowed to thaw to room temperature and then warmed at 42° C. for 5 minutes while still in their slide box. Slides were then removed from their box and baked an additional 10 minutes at 42° C. Slides were post-fixed 15 minutes in 4% paraformaldehyde/1% glutaraldehyde on ice followed by a 5 minutes rinse in 0.5×SSC. Sections were deproteinated in 4 μg/mL proteinase K for 30 minutes at 37° C., then washed for 10 minutes in 0.5×SSC. The slides were dehydrated with an ethanol gradient (70%-95%-100%) and air-dried. The slides were covered with 100 μl hybridization buffer (50% formamide, 10% dextran sulfate, and 2×SSC) and prehybridized for 1-4 hours at 42° C.

The [³³P]-labeled single-stranded actin RNA probe referenced above, at a concentration of 2×10⁶ cpm dissolved in 100 μl of hybridization buffer containing 1 mg/ml tRNA, was added to the prehybridization buffer on the slide, mixed well, covered with coverslip, and allowed to hybridize overnight at 55° C. in a sealed humidified container.

After hybridization, the slides were washed twice for 10 minutes in 2×SSC containing 1 mM EDTA at room temperature, and then incubated for 30 minutes at 37° C. in 20 μg/mL. RNase A in 10 mM Tris pH 8, 0.5 M NaCl. The slides were washed for 10 minutes in 2×SSC containing 1 mM EDTA at room temperature, then washed 4 times for 30 minutes each in 0.1×SSC containing 1 mM EDTA at 55° C., and then washed in 0.5×SSC for 10 minutes at room temperature. The slides were dehydrated for 2 minutes each in 50%, 70%, and 90% ethanol containing 0.3 M ammonium acetate, and allowed to air dry.

To view the results of the hybridization, the slides were exposed to a storage phosphor screen (Kodak) for 18 hours. The phosphor screen was scanned with a Typhoon 8600 Variable Mode Imager (Molecular Dynamics). The actin hybridization signal was detected in all the different spots on the frozen cell array described in Example 8. The intensity of the observed signal correlated with the number of cells loaded onto the array. Only the spot corresponding to the SKBr3 breast tumor cells lacked signal probably due to the loss of the element of the frozen cell array slide. These results illustrate that the frozen cell array of the invention provides good mRNA preservation and is sufficient to perform reliable in-situ hybridization procedures on many different cell lines at the same time.

### Example 10

### Ligand/Receptor Binding on a Frozen Cell Array

The present example demonstrates the utility of the invention for performing for ligand/receptor binding studies on a section of frozen cell array.

Microarray slides from the frozen cell array of Example 8 have been used to identify cells that express the IGF-1 receptor following a method described by Desnoyer L. et al., The journal of Histochemistry and Cytochemistry, Vol. 48, pp 1-9. Specifically, one or more frozen cell array sections of 10 μm thickness were applied to the Superfrost Plus Gold microscope slides (Ery Scientific, Portsmouth, N.H.), placed at room temperature for 30 seconds, and then stored at −20° C. for a minimum of 3 days before moving to storage at a temperature −70° C. The day of the ligand/receptor binding procedure, the frozen cell array slides were brought to room temperature and immediately incubated for 4 minutes in 35 mM acetic acid (pH 3.5) containing 3 mM CaCl₂, 3 mM MgSO₄, 5 mM KCl and 1 M NaCl. Subsequently, the slides were washed in HBS-C (25 mM Hepes, pH 7.2, 150 mM NaCl, 3 mM CaCl₂, 3 mM Mg SO₄, 5 mM KCl, complete protease inhibitor cocktail) containing 32 mM sucrose, and the nonspecific binding sites were blocked for 20 minutes in HBS-c containing 3% BSA and 32 mM sucrose. The binding sites for avidin and biotin were blocked using the avidin/biotin blocking kit from Vector (Burlingame, Calif.). The endogenous histidine-rich sites were blocked by incubating the slides for 10 minutes in 1 mM NiCl.

The frozen cell array slides were incubated for 1 hour in presence or absence 5 nM IGF-1-His tagged in HBS-C buffer containing 3% BSA and then washed three times for 1 minute each with cold HBS-C buffer containing 1% BSA. The slides were fixed in PBS containing 4% formaldehyde for 10 minutes and washed with HBS-C containing 1% BSA. The endogenous antibody binding sites were blocked with 1.5% normal horse serum in HBS-C for 20 minutes. The slides were then incubated with 1 mg/ml anti-H6 antibody (Roche Molecular Biochemicals, Indianapolis, Ind.) in HBS-C/3% BSA for 1 hour. Subsequently, the slides were washed with HBS-C/1% BSA and incubated for 30 minutes with biotinylated horse anti-mouse IgG (Vector, Burlingame, Calif.) diluted 1/200 in HBS-C containing 3% BSA. The slides were washed 3 times for 4 minutes and fixed in PBS/4% formaldehyde for 10 minutes. The slides were washed with HBS-C/1% BSA and incubated with streptavidin conjugated to horseradish peroxidase. The slides were washed 3 times for 1 minute in HBS-C/1% BSA and incubated for 10 minutes with biotin-conjugated tyramide (TSA) in NEN dilution buffer (NEN Life Science Products, Boston, Mass.). Alternatively, the slides could be incubated with TSA-Alexa 488 (Molecular Probes, Eugene, Oreg.) for 10 minutes. The reaction was stopped by 3 washes of 4 minutes in TBS/0.1% BSA. The slides previously incubated with biotin-conjugated TSA were incubated with streptavidin-conjugated FITC in TBS/0.1% BSA for 30 minutes.

Finally, the frozen cell array slides were washed 3 times for 1 minute each in TBS/0.05 Tween-20 and mounted using Vectashield mounting medium (Vector, Burlingame, Calif.) before being analyzed using a fluorescence microscope. The fluorescence signal detected on the HMVEC (human microvascular endothelial cells) and HUVEC (human umbilical vein endothelial cells) cell samples in the array slides in the presence or absence of IGF-1 using either the biotin-conjugated tyramide/streptavidin-conjugated FITC or the TSA-Alexa 488 signal system. Binding of IGF-1 on several cell types was detected in the frozen cell array, such as for example HMVEC and HUVEC cells, using either the biotin-conjugated tyramide/streptavidin-conjugated FITC or the TSA-Alexa 488 signal system, whereas binding was not observed in control cells lacking IGF-1.

This data illustrates that the frozen cell array described herein allows for good preservation of the native proteins at the cell surface compatible with ligand/receptor binding procedures on many different cell lines at the same time.

### Example 11

### Construction of a Frozen Tissue Array

The present example demonstrates the utility of the invention for constructing a frozen tissue array.

An arrayer having 25 40 mm×1.2 mm pins is constructed as described in Example 7. An OCT array recipient block is constructed as described in Example 7. Sample tissue is flash frozen in liquid nitrogen and stored at −70° C. The type of tissue sample can vary and includes normal or diseased tissue from human, murine, or other sources. The OCT array recipient block is removed from cold storage and placed onto a box filled with dry ice at room temperature. The array positions were numbered A-E for the columns and 1-5 for the rows for a total of 25 positions. Position A1 of the array was loaded with Trypan blue stain 0.4% (BivcoBRL) as a orientation marker. Position A1 of the array is loaded with Trypan blue stain 0.4% (BivcoBRL) as an orientation marker. Manual techniques or, alternatively, a tissue arrayer, such as a Beecher Instrument, with custom-size punches 1 mm in diameter, is used to extract frozen tissue samples from selected regions of the frozen tissue. The outer diameter of the tissue core should be the same size as the diameter of the well in the OCT array recipient block such that the tissue core fits tightly into the OCT well. Alternatively, the tissue cores could be submerged in an adhesive medium, such as an appropriate solvent, for example, ethanol, OCT diluted with ethanol, OCT diluted with propylene glycol, or propylene glycol (such that the adhesive medium has a freezing temperature approximately 5-10° C. below OCT), prior to being inserted into the OCT well, to promote adhesion of the frozen tissue core into the array. However, the adhesive medium must not exceed a temperature that would cause the frozen tissue to thaw. After the frozen tissue cores have been inserted into the frozen array recipient block forming a frozen array, the array is stored at −70° C. until sections are cut.

One or more sections of 6 μm to 12 μm thickness are cut from the frozen array on a cryostat and laid on pre-cleaned microscope slides (75 mm×25 mm, 0.96 to 1.09 mm thick)(Baxter Diagnostic Inc.). Each slide contains 2 sections of the frozen array (12 mm²) with each spot measuring 1 mm in diameter. The frozen tissue array slides are stored at −70° C. until analyzed.

### Example 12

### RNA/Agarose Internal Standard Preparation in the Frozen Tissue Array

The present example demonstrates the utility of the invention for quantitating biologically useful molecules, such as RNA, in a frozen tissue array using an internal standard preparation having a known quantity of the biological molecule in a solid embedding material, such as agarose.

A frozen OCT array recipient block is made using the procedure described in Example 7; the frozen recipient block is stored at −70° C. until loaded. Her2/ErbB2 RNA is transcribed in-vitro using the procedure described in Example 1.

A working concentration of 100 ng/μl of the Her2/ErbB2 RNA solution is made by adding 50 μl of the RNA Solution and 200 μl TE to a new Eppendorf tube. The Eppendorf tube is heated in a 95° C. heat block for 3 minutes to denature the RNA transcript and then chilled immediately on ice. To the RNA Solution, 250 μl of 8% NuSieve 3:1 (a high gel strength agarose melted at 99° C.) and 500 μl SQH₂O that has been warmed in a 50° C. heat block is added. The RNA/agarose mixture is vortexed briefly and then poured into a 15 mm×15 mm DisPO base mold (Baxter Scientific). The final concentration of the RNA in the internal standard preparation is 5 μg/ml. The RNA/Agarose mixture is then allowed to gel at 4° C. for at least one hour. After the gel forms, the RNA/agarose blocks are removed from the plastic molds using a clean razor blade. A punch or arraying instrument, such as a tissue arrayer, is used to extract cores from the RNA/agarose block as described herein. A similar arraying instrument may also be used to insert cores of tissue test samples into other wells of the array as described in Example 11. Alternatively, the RNA/agarose mixture could be pipetted before it forms a gel into one or more wells of the OCT array recipient block. Cores could also possibly be made from the solidified cooled RNA/agarose blocks. The array recipient block with the internal standard preparation is stored at −70° C. until samples are loaded.

### Example 13

### Protein/Agarose Internal Standard Preparation in the Frozen Cell Array

The present example demonstrates the utility of the invention for quantitating biologically useful molecules, such as proteins, in a frozen cell array using an internal standard having a known quantity of the biological molecule in a solid embedding material, such as agarose.

A frozen OCT array recipient block is made using the procedure described in Example 7; the frozen recipient block is stored at −70° C. until loaded. A Her2/ErbB2 protein/agarose internal standard preparation is made using the procedure described in Example 2, except that after the protein and agarose are mixed, the mixture is partially cooled such that it is warm enough to be poured but cool enough not to cause the frozen OCT to melt.

The OCT array recipient block is removed from cold storage and placed onto a box filled with dry ice at room temperature. To load the cells, the OCT array is removed from cold storage and placed onto a box filled with dry ice at room temperature. The array positions are numbered A-E for the columns and 1-5 for the rows for a total of 25 positions. Position A1 is loaded with Trypan blue stain 0.4% (BivcoBRL) as a orientation marker. One or more wells in column A of the OCT array recipient block are loaded with the protein/agarose internal standard preparation using 1 ml syringes with 22_{G}1.5 inches long (0.7 mm×40 mm) needles (Becton Dickinson & Co.). Other wells of the OCT array recipient block are loaded with cell suspensions as described in Example 7. The frozen array is stored at −70° C. until sectioning.

Alternatively, the protein/agarose mixture is then allowed to gel at 4° C. for at least one hour. After the gel forms, the protein/agarose blocks are removed from the plastic molds using a clean razor blade. A punch or arraying instrument, such as a tissue arrayer, is used to extract cores from the protein/agarose block. A similar arraying instrument may also be used to insert cores of tissue test samples into other wells of the array.

### Example 14

### Cellulose/Agarose Internal Standard Preparation Orientation Marker

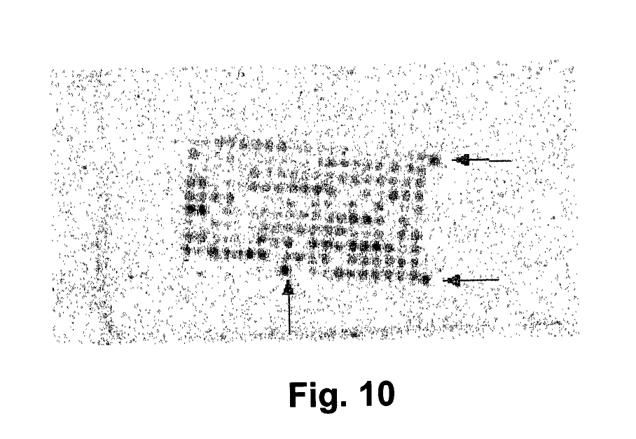

The present example demonstrates the utility of the invention for including an orientation marker in an array consisting of a non-specific binder of radioactive and/or fluorescent probes, such as cellulose, in an embedding material, such as agarose. Specifically, the non-specific binder of an isotopically labeled hybridization probe, when viewed on a phosphorimager image, allows the unambiguous orientation of other signals in an array. A first internal standard preparation was made using microgranular cellulose as a non-specific binder of a standard molecule as described herein. To synthesize the non-specific binder containing approximately 20% weight/volume of cellulose, approximately 1 g of microgranular cellulose (Sigma Chem. Co. Cat. #C-6413) was added to 3 ml of H₂O and mixed to form a suspension. A 750 μl aliquot of the cellulose suspension was added to 250 μl of 8% NuSieve 3:1 agarose (a high gel strength agarose melted at 99° C.), vortexed, and poured into a 15 mm×15 mm DisPO base mold (Baxter Scientific). The first internal standard preparation was allowed to gel at 4° C. for at least one hour. The tissue microarray shown in FIG. 10 comprising orientation markers demonstrates non-specific binding of labeled polynucleotide probe to the non-specific binding material, microgranular cellulose, in the markers. The TMA orientation markers are indicated with arrows in the phosphorimager scan of FIG. 10. The cellulose cores consistently bound probe non-specifically, permitting unambiguous alignment of positive elements in relation to the orientation markers.

A second internal standard preparation was made using fibrillar cellulose as a non-specific binder of standard molecule as described herein. To synthesize the non-specific binder containing approximately 20% weight/volume of cellulose, approximately 1 g of fibrillar cellulose (Sigma Chem. Co. Cat. #C-6288) was added to 3 ml of H₂O and mixed to form a suspension. A 750 μl aliquot of the cellulose suspension was added to 250 μl of 8% NuSieve 3:1 agarose (a high gel strength agarose melted at 99° C.), vortexed, and poured into a 15 mm×15 mm DisPO base mold (Baxter Scientific). The second internal standard preparation was allowed to gel at 4° C. for at least one hour.

After the gel was formed, each of the first and second internal standard preparation/orientation markers were removed from the plastic molds using a clean razor blade and the intact blocks were fixed in 10% neutral buffered formalin overnight at room temperature. The agarose blocks were then transferred to 70% ethanol and processed using standard techniques for paraffin embedding as described in Example 1.

A biological array made of a paraffin block having one row of nine wells was made as described herein by inserting three cores of the first internal standard preparation into the first three wells of the array, three cores of agarose alone into the middle three wells of the array, and three cores of the second internal standard preparation in the last three wells of the array. Four slices of equal thickness perpendicular to the foregoing nine cores were cut from the array, and each of the four slices was mounted on a glass slides as described in Examples 3 and 4 to form four microarrays.

Anti-sense and sense probes for Her2/ErbB2 RNA were prepared with [³³P]-label as described in Example 3. The sequence of the PCR amplified DNA template (sense orientation) used to transcribe the RNA probes was:

| | |
|---|---|
| TGGTCGTGGTCTTGGGGGTGGTCTTTGGGATCCTCATCAAGCGACGGCAGCAG | [SEQ ID NO: 8] |
| AAGATCCGGAAGTACACGATGCGGAGACTGCTGCAGGAAACGGAGCTGGTGGA | |
| GCCGCTGACACCTAGCGGAGCGATGCCCAACCAGGCGCAGATGCGGATCCTGA | |
| AAGAGACGGAGCTGAGGAAGGTGAAGGTGCTTGGATCTGGCGCTTTTGGCACA | |
| GTCTACAAGGGCATCTGGATCCCTGATGGGGAGAATGTGAAAATTCCAGTGGC | |
| CATCAAAGTGTTGAGGGAAAACACATCCCCCAAAGCCAACAAAGAAATCTTAG | |
| ACGAAGCATACGTGATGGCTGGTGTGGGCTCCCCATATGTCTCCCGCCTTCTG | |
| GGCATCTGCCTGACATCCACGGTGCAGCTGGTGACACAGCTTATGCCCTATGG | |
| CTGCCTCTTAGACCATGTCCGGGAAAACCGCGGACGCCTGGGCTCCCAGGACC | |
| TGCTGAACTGGTGTATGCAGATTGCCAAGGGGATGAGCTACCTGGAGGATGTG | |
| CGGCTCGTACACAGGGACTTGGCCGCTCGGAACGTGCTGGTCAAGAGTCCCAA | |
| CCATGTCAAAATTACAGACTTCGGGCTGGCTCGGCTG | |

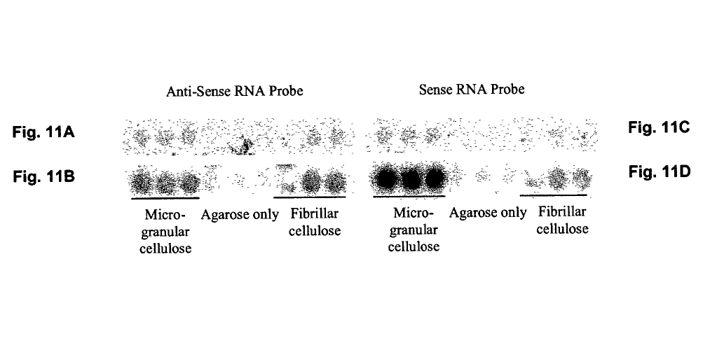

Two of the microarrays were hybridized to the anti-sense probe and two of the microarrays were hybridized to the sense probe. As can be seen in FIGS. 11A-11D, both sense and anti-sense Her2/ErbB2 probes bound detectably to the cellulose-containing internal standard preparations. FIGS. 11A and 11B show the autofluorescence (excitation frequency=532 nm, emission filter set=610 nm/bandpass 30 nm) for one microarray hybridized to the anti-sense probe and one microarray hybridized to the sense probe, respectively. A phosphoroimager was used to review the hybridization results of the remaining two microarrays as described in Example 3, the results of which are shown in FIGS. 11C and 11D for the antisense and sense probes, respectively. The fact that the cellulose-containing internal standard preparations can be visualized both by their autofluorescence and by their non-specific binding of labeled probe allows these internal standard preparations to be used as positional markers to register the ISH phosphorimager signals with the core positions, even when only a few clinical sample cores are visible in the ISH phosphorimager signal.

### Example 15

### Dye/Agarose Internal Standard Preparation Orientation Marker

The present example demonstrates the utility of the invention for including an internal standard preparation/orientation marker in an array consisting of a dye in an embedding material, such as agarose.

Four volumes of 250 μl of black, blue, and yellow surgical marking dye, (Triangle Biomedical Sciences, Durham, N.C.), respectively, were added to an Eppendorf tube. To each of the dyes, 750 μl of 2% NuSieve 3:1 agarose (a high gel strength agarose melted at 99° C.) was added, and the mixture was vortexed and poured into a 15 mm×15 mm DisPO base mold (Baxter Scientific). Each of the four internal standard preparations/orientation markers were then allowed to gel at 4° C. for at least one hour.

A typical tissue array containing clinical prostate cancer samples and internal colored marker dye standard preparations was constructed containing 240 cores arrayed in 20 columns and 12 rows as follows. Two hundred thirty-two sample tissue cores measuring 0.6 mm in diameter were obtained from various donor paraffin blocks. The donor blocks included 57 specimens of prostatic adenocarcinoma tissue, 22 specimens of which had adjacent normal prostate tissue sampled; each donor block area (tumor and normal) was sampled in duplicate or triplicate (University of Sheffield, England). The sample cores were embedded into a recipient paraffin block, for example, using a Beecher tissue arraying instrument, as described in Example 3.

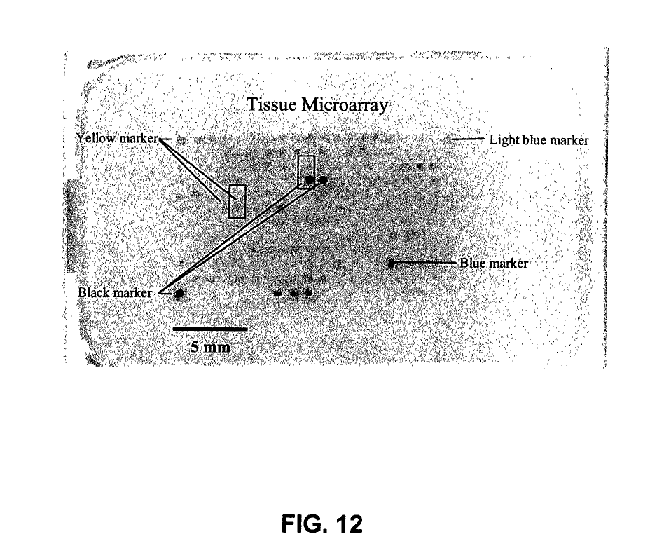

Eight cores of Internal Colored Dye Standard Preparations, measuring 0.6 mm in diameter were obtained from donor blocks containing dye/agarose, each prepared as described above. Three cores from the black dye/agarose block, three cores from the yellow dye/agarose block, 1 core from the blue dye/agarose block, and one core from the light blue dye/agarose block were inserted into the array recipient block array in an asymmetrical pattern as shown in FIG. 12. The dye/agarose internal standard preparation is very clear and stands out appreciably in the array, thereby allowing for unambiguous orientation when viewing the array.

All of the cores were annealed in the array recipient block array by incubating the block in a 37° C. oven overnight. For analysis, the paraffin array was sliced into 3-5 μm thick histological TMA sections. Each TMA section was then transferred into a 42° C. water bath, collected individually onto Superfrost glass slides, and thoroughly dried. The TMA section was deparaffinized, and stained with hematoxylin and eosin using similar procedures as described herein. Following this procedure, the dye/agarose orientation markers continued to be clearly observable and to stand out appreciably as compared to the tissue samples in the array.

### Example 16

### Red Blood Cell Ghosts and RNA/Agarose Internal Standard Preparation

The present example demonstrates the utility of the invention for utilizing red blood cell ghosts to entrap RNA and/or protein internal standards admixed in an embedding material, such as agarose, for use in a tissue or cell array. Introducing RNA and protein standards into such a vehicle more closely mimics conditions within a tissue, which may influence hybridization kinetics and antibody access or recognition.

### Red Blood Cell Lysis

Red blood cell ghosts are prepared according the method of Boogaard and Dixon, Procedural Cell Research 143:175-190 (1983). Briefly, ten milliliters of heparinized blood is centrifuged at 2300×g for 10 min at 4° C. After centrifugation, the serum and white cells are aspirated. The red blood cells are washed three times by suspension in 10 ml of cold Hanks Balanced Salt Solution (HBSS). The red blood cells are centrifuged again and any remaining white cells are removed with the supernatant. After removal of the white cells after the third wash, 20 ml 55% HBSS is added to the red blood cells to cause them to swell. The swollen red blood cells are centrifuged and the supernatant is removed leaving a swollen red blood cell pellet. The following are added to a tube to initiate lysis: 2 ml of the swollen red cell pellet; 10 ml of 20% HBSS; and 2 ml of 10 mM Tris-HCl with a pH of 7.6. The tube is inverted several times and lysis is allowed to proceed for 2 min at 4° C. to allow the cellular contents (e.g., endogenous proteins and residual RNA) to leak from the red blood cells. After 2 min., 1.5 ml of 10×HBSS is added to the suspension in the tube to close the holes in the membranes of the red blood cells caused by lysis. The suspension in the tube is incubated in a 37° C. waterbath for 1 hour to reseal the membranes and then centrifuged at 4° C. to remove the supernatant.

The foregoing lysis procedure can be repeated two or three times as desired. After the final resealing step, the volume of the swollen cell pellet is reduced by careful aspiration to 0.2 ml and the cells are ready for loading.

### Red Blood Cell Loading

To make loading buffer, RNA is suspended in 10 mM Tris-HCl, pH 7.0, 5 mM DTT at a concentration of approximately 1 mg/ml. A 200 μl aliquot of loading buffer at 4° C. is added to a tube with 200 μl of the swollen red cells and the tube is vortexed for 2 minutes in order to maximize RNA uptake by permeable red cells. A 30 μl aliquot of 10×HBSS is added to the tube. The tube is vortexed and incubated for 30-45 min in a 37° C. waterbath to seal the RNA-loaded cells. After incubation, the red blood cells are returned to 4° C. and washed by adding 150 mM NaCl, 10 mM Tris at pH 7.0, and 5 mM DTT, to create a cell suspension with a volume of 10 ml. The cell suspension is centrifuged as described above for 25 min to create a cell pellet. The cell pellet is washed two more times with 150 mM NaCl, 10 mM Tris at pH 7.0, and 5 mM DTT.

The cell pellet is then gently removed from the tube and added to 2% NuSieve 3:1 agarose (a high gel strength agarose melted at 99° C., then cooled to about 60° C.) in a 15 mm×15 mm DisPO base mold (Baxter Scientific). The mixture is then allowed to gel at 4° C. for at least one hour. Protein is loaded into the red blood cell ghosts in the same manner except that protein solution is resuspended in 10 mM Tris-HCl, pH 7.0, 5 mM DTT at the desired concentration, and a 200 μl aliquot of protein/loading buffer at 4° C. is added to a tube with 200 μl of the swollen red cells.

After the gel is formed, the protein and/or RNA-loaded red blood cell ghost/agarose block is removed from the plastic mold using a clean razor blade and the intact block is fixed in 10% neutral buffered formalin overnight at room temperature as described in Example 1. The agarose block is then transferred to 70% ethanol and processed using standard techniques for paraffin embedding as described in Examples 1 and used as described in Examples 3 and 4.

### Example 17

### Construction of a Frozen Cell Array

The present example demonstrates the utility of the invention for constructing a frozen tissue or cell array.

An arrayer is made having 56 metal pins, measuring 40 mm long×1.2 mm in diameter, that are heat-sealed and glued with Epoxy in a base made of Plexiglas measuring 25 mm×25 mm. The pins are arranged in seven rows and eight columns, with each pin being equally spaced approximately 1 mm apart. The total area of the pins is 20 mm×22 mm, thereby making the density of pins about 13 pins/cm². A fluid OCT medium is poured into a disposable embedding mold (VWR) measuring 22 mm×30 mm×20 mm (deep). The arrayer pins are first immersed in glycerol and then partially immersed in a fluid OCT medium contained within the embedding mold. The fluid OCT is frozen by submerging the fluid OCT, the mold, and the engaged pins in a cryobath of isopentane at −160° C. for 3 to 5 minutes. The pins are then extracted from the OCT mold leaving an array of 56 wells no more than 20 mm deep in an array recipient block, with about 13 well/cm². The array recipient block is stored at −70° C. until the wells are loaded with various cell lines.

The 56 wells of the array recipient block are loaded with one or more biological samples to create a frozen biological array as described in Examples 7 or 9-11. The frozen biological array is cut for slides into sections in a range of 6 μm to 12 μm thickness using a cryostat or other slicing instrument. Two sections from the frozen array are laid onto a pre-cleaned microscope slides measuring 75 mm×25 mm, 0.96 to 1.09 mm thick (Baxter Diagnostic Inc.). Each slide contains 112 spots of sample corresponding to the 56 wells in the array, with each spot measuring approximately 1.1 mm in diameter. The cell array slides were stored at −70° C. until used for analysis.

### Deposit of Materials

The following hybridoma cell line has been deposited with the American Type Culture Collection, 10801 University Boulevard, Manassus, Va. 20110-2209, USA (ATCC):

| Antibody Designation | ATCC No. | Deposit Date |
|---|---|---|
| 4D5 | ATCC CRL 10463 | May 24, 1990 |

The deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and is subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

We claim:

1. A method for preparing an array recipient block comprising:
engaging an arrayer having a plurality of pins with an embedding mold and a fluid temperature-sensitive matrix such that the matrix and the pins are contained within the embedding mold, wherein the embedding mold has a bottom surface;
freezing the matrix within the embedding mold to solidify the matrix; and
removing the arrayer pins from the matrix and embedding mold to form a plurality of wells disposed within the solid temperature-sensitive matrix.

2. The method of claim 1, wherein the arrayer further comprises a body, the plurality of pins protrude from the body, and each of the plurality of pins has a first end affixed to the body and a free end opposite the first end.

3. The method of claim 2, wherein the engaging step further comprises fully inserting the arrayer pins into the embedding mold such that the free end of each of the pins touches the bottom surface of the mold.

4. The method of claim 3, wherein the free end of each of the plurality of pins is tapered to form a point.

5. The method of claim 3, wherein the free end of each of the plurality of pins comprises a needle.

6. The method of claim 2, wherein the engaging step further comprises partially inserting the arrayer pins into the embedding mold such that the free end of each of the pins does not touch the bottom surface of the mold.

7. The method of claim 1, wherein the temperature-sensitive matrix comprises resin-polyvinyl alcohol and polyethylene glycol.

8. The method of claim 1, wherein the engaging step further comprises coating the arrayer pins with a lubricating material.

9. The method of claim 8, wherein the lubricating material is selected from a group consisting of glycerol, fatty acids, oil, grease, fat, or soap.

10. The method of claim 1, wherein the freezing step comprises contacting the embedding mold, the fluid temperature-sensitive matrix, and the arrayer pins with an environment, wherein the temperature of the environment is below the freezing temperature of the temperature-sensitive matrix.

11. The method of claim 10 wherein the environment is a temperature is at least 3° C., at least 5° C., or at least 10° C. below the freezing temperature of the temperature-sensitive matix.

12. The method of claim 11, wherein the environment is liquid isopentane.

13. The method of claim 12, wherein the isopentane has a temperature of about −160° C.

14. The method of claim 1, wherein the freezing temperature of the temperature-sensitive matrix is in a range of about −10° C. to about −50° C., about −20° C. to about −50° C., about −20° C. to about −35° C., about −35° C. to about −50° C., about −10° C. to about −35° C., or about −10° C. to about −20° C.

15. The method of claim 1, wherein the temperature-sensitive matrix is Optimal Cutting Temperature material (OCT).

16. A biological array comprising:
a frozen matrix formed of a temperature-sensitive material having a plurality of wells disposed therein; and
one or more biological samples disposed within the plurality of wells and retained by the frozen matrix surrounding the wells, wherein the freezing temperature of the temperature-sensitive material is lower than the freezing temperature of the biological samples.

17. The biological array of claim 16, wherein the temperature-sensitive material comprises resin-polyvinyl alcohol and polyethylene glycol.

18. The biological array of claim 16, wherein the temperature-sensitive matrix material is OCT.

19. The biological array of claim 16, further comprising more than 5 wells/cm².

20. The biological array of claim 19, wherein the cross sectional diameter of one or more of the wells is in a range of about 0.4 mm to about 1.2 mm, about 0.4 mm to about 0.7 mm, or about 0.8 mm to about 1.2 mm.

21. The biological array of claim 16, wherein one or more of the biological samples comprise cells.

22. The biological array of claim 21, wherein the cells are selected from the group consisting of normal cells, diseased cells, and treated cells.

23. The biological array of claim 21, wherein one or more of the biological samples comprise a cell suspension or comprise a tissue.

24. The biological array of claim 23, wherein the tissue is selected from the group consisting of blood, muscle, nerve, brain, breast, prostate, heart, lung, liver, pancreas, spleen, thymus, esophagus, stomach, intestine, kidney, testis, ovary, uterus, hair follicle, skin, bone, bladder, and spinal cord.

25. The biological array of claim 23, wherein the tissue is selected from the group consisting of normal tissue, diseased tissue, and tissue comprising cancerous cells.

26. The biological array of claim 25, wherein the tissue is from an organism selected from the group consisting of an adult organism and an organism at a pre-adult stage of development.

27. The biological array of claim 16, further comprising one or more internal standard preparations disposed within the plurality of wells, wherein the internal standard preparation comprises a standard molecule admixed with an embedding material and the embedding material differs from the matrix material in at least one physical or chemical property.

28. The array of claim 27, wherein the standard molecule is selected from the group consisting of a polynucleotide, an RNA molecule, a DNA molecule, and a polypeptide.

29. The array of claim 27, wherein the internal standard preparation further comprises two or more different standard molecules.

30. The array of claim 29, wherein one of the standard molecules is a polynucleotide and one of the standard molecules is a polypeptide.

31. The array of claim 29, wherein the internal standard preparation comprises two or more different polynucleotides.

32. The array of claim 29, wherein the internal standard preparation comprises two or more different polypeptides.

33. The array of claim 27, wherein the embedding material comprises agarose.

34. The array of claim 33, wherein the embedding material comprises agarose at a concentration of about 1% to about 3% agarose, about 1.5% to about 2.5% agarose, or about 1.8% to about 2.2% agarose, or about 2% agarose.

35. The array of claim 33, wherein the embedding material further comprises about 0.5% to about 10% bovine serum albumin (BSA), about 1% to about 7% BSA, about 1% to about 6% BSA, or about 1% to about 5% BSA.

36. The array of claim 27, wherein the internal standard preparation further comprises about 0.5% to about 20% bovine serum albumin (BSA), about 1% to about 15% BSA, about 1% to about 10% BSA, or about 1% to about 5% BSA.

37. The array of claim 27, further comprising two or more internal standard preparations, wherein at least two of the internal standard preparations comprise different concentrations of a standard molecule admixed in the embedding material.

38. The array of claim 27, further comprising an array orientation marker within one or more of the plurality of wells.

39. The array of claim 27, wherein the internal standard preparation comprises a known quantity of the standard molecule.

40. The array of claim 16, wherein each of the plurality of wells are lined with a lubricating material.

41. The array of claim 40, wherein the lubricating material is selected from a group consisting of glycerol, fatty acids, oil, grease, fat, and soap.

42. An apparatus for preparing an array for biological samples comprising:
an arrayer having a body and a plurality of pins protruding from the body, wherein each pin has a first end affixed to the body and a free end opposite the first end;
an embedding mold having a bottom surface; and
a temperature-sensitive matrix contained within the embedding mold, wherein the temperature-sensitive matrix has a freezing temperature below a freezing temperature of the biological samples.

43. The apparatus of claim 42 wherein the freezing temperature of the temperature-sensitive matrix is at least 3° C., at least 5° C., or at least 10° C. below the freezing temperature of the biological samples.

44. The apparatus of claim 42, wherein the temperature-sensitive matrix comprises resin-polyvinyl alcohol and polyethylene glycol.

45. The apparatus of claim 44, wherein the temperature-sensitive matrix is Optimal Cutting Temperature material (OCT).

46. The apparatus of claim 42, wherein the arrayer body is formed from a rigid material selected from a group consisting of Plexiglas, plastic, ceramic, glass, metal, and wood.

47. The apparatus of claim 42, wherein the arrayer comprises more than 5 pins/cm², more than 7 pins/cm², or more than 13 pins/cm².

48. The apparatus of claim 42, wherein the free end of one or more of the plurality of pins is tapered to form a point.

49. The apparatus of claim 42, wherein the free end of one or more of the plurality of pins has a diameter less than the diameter of the pin.

50. The apparatus of claim 42, wherein one or more of the plurality of pins comprises a glass blunt.

51. The apparatus of claim 50, wherein the free end of the glass blunt is closed with a sealer.

52. The apparatus of claim 51, wherein a needle protrudes from the sealer within the free end of the glass blunt.

53. The apparatus of claim 42, wherein one or more of the plurality of pins comprises a solid lumen.

54. The apparatus of claim 42, wherein one or more of the plurality of pins comprises a hollow lumen and is sealed at the free end.

55. The apparatus of claim 42, wherein one or more of the plurality of pins have a circular cross-sectional shape.

56. The apparatus of claim 55, wherein one or more of the plurality of pins has a cross sectional diameter in a range of about 0.4 mm to about 1.2 mm, about 0.4 mm to about 0.7 mm, or about 0.8 mm to about 1.2 mm.

57. A biological array comprising:
a matrix having a plurality of wells disposed therein;
one or more biological samples contained in one or more of the plurality of wells; and
one or more internal standard preparations contained in one or more of the plurality of wells, the internal standard preparation comprising a standard molecule admixed in an embedding material, wherein the embedding material differs from the matrix in at least one physical or chemical property.

58. The biological array of claim 57, wherein the standard molecule is selected from the group consisting of a polynucleotide, an RNA molecule, a DNA molecule, and a polypeptide.

59. The biological array of claim 58, wherein the standard molecule is a polynucleotide comprising at least 20 contiguous nucleotides of the Her2 gene or VEGF gene or their complementary sequences.

60. The biological array of claim 58, wherein the standard molecule is a polypeptide selected from the group consisting of a receptor, a soluble receptor, a receptor extracellular domain (ECD), a ligand-binding fragment of a receptor, a receptor ligand, an antibody, an antigen-binding fragment of an antibody, an antigen, HER2, VEGF, and a fragment HER2 or VEGF comprising at least 10 contiguous amino acids of HER2 polypeptide or VEGF polypeptide.

61. The biological array of claim 57, wherein the internal standard preparation further comprises two or more different standard molecules.

62. The biological array of claim 61, wherein one of the standard molecules is a polynucleotide and one of the standard molecules is a polypeptide.

63. The biological array of claim 61, wherein the internal standard preparation comprises two or more different polynucleotides.

64. The biological array of claim 61, wherein the internal standard preparation comprises two or more different polypeptides.

65. The biological array of claim 57, wherein the embedding material comprises agarose.

66. The biological array of claim 65, wherein the embedding material comprises agarose at a concentration of about 1% to about 3% agarose, about 1.5% to about 2.5% agarose, or about 1.8% to about 2.2% agarose, or about 2% agarose.

67. The biological array of claim 65, wherein the embedding material further comprises about 0.5% to about 10% bovine serum albumin (BSA), about 1% to about 7% BSA, about 1% to about 6% BSA, or about 1% to about 5% BSA.

68. The biological array of claim 57, wherein the internal standard preparation further comprises about 0.5% to about 20% bovine serum albumin (BSA), about 1% to about 15% BSA, about 1% to about 10% BSA, or about 1% to about 5% BSA

69. The biological array of claim 57, wherein the sample is a tissue.

70. The biological array of claim 69, wherein the tissue is selected from the group consisting of blood, muscle, nerve, brain, breast, prostate, heart, lung, liver, pancreas, spleen, thymus, esophagus, stomach, intestine, kidney, testis, ovary, uterus, hair follicle, skin, bone, bladder, and spinal cord.

71. The biological array of claim 69, wherein the tissue is selected from the group consisting of normal tissue, diseased tissue, tissue from an adult organism, and tissue from an organism at a pre-adult stage of development.

72. The biological array of claim 57, wherein the sample is a cell suspension.

73. The biological array of claim 57, wherein the matrix comprises a temperature-sensitive material selected from the group consisting of paraffin, gelatin, and Optimal Cutting Temperature material (OCT).

74. The biological array of claim 57, further comprising two or more internal standard preparations, wherein at least two of the internal standard preparations comprise different concentrations of the standard molecule admixed in the embedding material.

75. The biological array of claim 57, further comprising an array orientation marker within one or more of the plurality of wells.

76. A method of making a biological array comprising:
preparing a matrix having a plurality of wells disposed therein;
mixing a standard molecule with an embedding material to form an internal standard preparation, wherein the embedding material differs from the matrix in at least one physical or chemical property;
inserting the internal standard preparation into one or more of the plurality of wells in the matrix; and
inserting a sample into one or more of the plurality of wells in the matrix.

77. The method of claim 76, wherein the matrix comprises a temperature-sensitive material selected from the group consisting of paraffin, gelatin, a material comprising resin-polyvinyl alcohol and polyethylene glycol, and Optimal Cutting Temperature material (OCT).

78. The method of claim 77, wherein the preparing step further comprises forming wells in the matrix.

79. The method of claim 76, wherein the preparing step further comprises:
engaging a plurality of pins with an embedding mold and a fluid temperature-sensitive matrix such that the matrix and the pins are contained within the embedding mold;
freezing the matrix within the embedding mold to solidify the matrix; and
removing the pins from the matrix and embedding mold to form a plurality of wells disposed within the solid temperature-sensitive matrix.

80. The method of claim 79, wherein the preparing step further comprises lubricating the plurality of pins prior to engaging the plurality of pins with the embedding mold and the fluid temperature-sensitive matrix.

81. The method of claim 76, wherein the standard molecule is selected from the group consisting of a polynucleotide, an RNA molecule, an in vitro transcribed RNA molecule, a DNA molecule, a polynucleotide comprising at least 20 contiguous nucleotides of the Her2 gene or VEGF gene or their complementary sequences, a polypeptide, and a polypeptide comprising at least 10 contiguous amino acids of the HER2 polypeptide or the VEGF polypeptide.

82. The method of claim 76, wherein the mixing step further comprises mixing a plurality of standard molecules in the embedding material to form the internal standard preparation.

83. The method of claim 82, wherein the mixing step further comprises mixing one or more polynucleotides and one or more polypeptides with the embedding material to form the internal standard preparation.

84. The method of claim 82, wherein the mixing step further comprises mixing two or more different polynucleotides with the embedding material to form the internal standard preparation.

85. The method of claim 82, wherein the mixing step further comprises mixing two or more different polypeptides with the embedding material to form the internal standard preparation.

86. The method of claim 76, wherein the mixing step comprises mixing the standard molecule with agarose to form the internal standard preparation.

87. The method of claim 86, wherein the agarose concentration in the internal standard is about 1% to about 3% agarose, about 1.5% to about 2.5% agarose, or about 1.8% to about 2.2% agarose, or about 2% agarose.

88. The method of claim 76, wherein the mixing step comprises mixing the standard molecule with agarose and bovine serum albumin (BSA) to form the internal standard preparation.

89. The method of claim 76, wherein the BSA concentration in the internal standard preparation is about 0.5% to about 20% bovine serum albumin (BSA), about 1% to about 15% BSA, about 1% to about 10% BSA, or about 1% to about 5% BSA.

90. The method of claim 76, wherein the mixing step further comprises pouring the internal standard preparation into a mold and allowing the internal standard preparation to solidify and form an internal standard donor block.

91. The method of claim 90, wherein the inserting the internal standard preparation step comprises punching a core from the internal standard donor block and inserting the core into one or more of the plurality of wells in the matrix.

92. The method of claim 76, wherein the step of inserting the internal standard comprises pouring the internal standard preparation into one or more of the plurality of wells in the matrix.

93. A method for detecting a biological molecule in an array, the method comprising:
mixing a known quantity of the biological molecule with an embedding material so as to provide an internal standard preparation;
inserting the internal standard preparation into one or more of a plurality of the wells in an array recipient block, the array recipient block comprising a matrix that differs from the embedding material by one or more physical or chemical properties;
inserting one or more samples into one or more of the plurality of wells in the array recipient block to form an array;
performing an analytical procedure on the array; and
correlating a result of the analytical procedure on the internal standard preparation to a result of the analytical procedure on the sample to determine detection of the biological molecule in the sample.

94. The method of claim 93, wherein the biological molecule is a polynucleotide selected from the group consisting of an RNA molecule, a DNA molecule, and a polynucleotide comprising at least 20 contiguous nucleotides of the Her2 gene or at least 20 contiguous nucleotides of the VEGF gene or their complementary sequences.

95. The method of claim 93, wherein the biological molecule is a polypeptide.

96. The method of claim 93, wherein the biological molecule is selected from the group consisting of a receptor, a soluble receptor, a receptor extracellular domain (ECD), a ligand-binding fragment of a receptor, a receptor ligand, an antibody, an antigen-binding fragment of an antibody, an antigen, and a polypeptide comprising at least 10 contiguous amino acids of HER2 polypeptide or at least 10 contiguous amino acids of VEGF.

97. The method of claim 93, wherein the internal standard preparation further comprises two or more different biological molecules.

98. The method of claim 97, wherein one of the biological molecules is a polynucleotide and one of the biological molecules is a polypeptide.

99. The method of claim 97, wherein the internal standard preparation comprises two or more different polynucleotides.

100. The method of claim 97, wherein the internal standard preparation comprises two or more different polypeptides.

101. The method of claim 93, wherein the embedding material comprises agarose.

102. The method of claim 101, wherein the agarose concentration in the internal standard is about 1% to about 3% agarose, about 1.5% to about 2.5% agarose, or about 1.8% to about 2.2% agarose, or about 2% agarose.

103. The method of claim 93, wherein the internal standard preparation further comprises bovine serum albumin (BSA), and wherein the BSA concentration in the internal standard preparation is about 0.5% to about 20% bovine serum albumin (BSA), about 1% to about 15% BSA, about 1% to about 10% BSA, or about 1% to about 5% BSA.

104. The method of claim 93, wherein the sample comprises a tissue.

105. The method of claim 104, wherein the tissue is selected from the group consisting of blood, muscle, nerve, brain, breast, prostate, heart, lung, liver, pancreas, spleen, thymus, esophagus, stomach, intestine, kidney, testis, ovary, uterus, hair follicle, skin, bone, bladder, and spinal cord.

106. The method of claim 104, wherein the tissue is selected from the group consisting of normal tissue, diseased tissue, tissue from an adult organism, and tissue from an organism in a pre-adult stage of development.

107. The method of claim 93, wherein the sample comprises a cell suspension.

108. The method of claim 93, wherein the matrix comprises a temperature-sensitive material selected from the group consisting of paraffin, gelatin, a material comprising resin-polyvinyl alcohol and polyethylene glycol, and Optimal Cutting Temperature material (OCT).

109. The method of claim 93, wherein the analytical procedure comprises in-situ hybridization.

110. The method of claim 93, wherein the analytical procedure comprises immunohistochemistry.

111. The method of claim 93, wherein the analytical procedure comprises immunofluorescence.

112. The method of claim 93, wherein the biological molecule is a receptor and the analytical procedure comprises contacting a ligand with the receptor and detecting binding of the ligand and the receptor.

113. The method of claim 112, wherein the ligand is detectably labeled.

114. The method of claim 93, wherein the biological molecule is a ligand and the analytical procedure comprises contacting a ligand-binding polypeptide with the ligand and detecting binding of the ligand and the ligand-binding polypeptide.

115. The method of claim 114, wherein the ligand-binding polypeptide is selected from the group consisting of a receptor, a ligand-binding fragment of a receptor, an receptor ECD, a ligand-specific antibody, a ligand-specifc binding fragment of an antibody.

116. The method of claim 115, wherein the antibody is anti-HER2 or anti-VEGF.

117. The method of claim 115, wherein the ligand-binding polypeptide is detectably labeled.

118. The method of claim 93, wherein the analytical procedure comprises contacting a detectably labeled compound with the biological molecule.

119. The method of claim 118, wherein the detectably labeled compound is selected from a group consisting of a labeled polynucleotide probe or a labeled polypeptide.

120. The method of claim 119, wherein the labeled polypeptide is selected from the group consisting of an antibody, a monoclonal antibody, a ligand-binding fragment of an antibody, a receptor, a receptor ECD, a ligand-binding fragment of a receptor, an anti-HER2 antibody, an anti-VEGF antibody, a ligand-binding antibody fragment of an anti-HER2 antibody, a ligand-binding fragment of an anti-VEGF antibody, a HER2 receptor, a VEGF receptor, a ligand-binding fragment of a HER2 receptor, and a ligand-binding fragment of a VEGF receptor.

121. The method of claim 119, wherein the correlating step comprises determining the amount of detectably labeled compound bound to an internal standard preparation relative to the amount of the detectably labeled compound bound to a sample.

122. The method of claim 118, wherein the detectably labeled compound comprises a label selected from the group consisting of a radioisotope, a chemiluminescent label, a luminescent label, a fluorophore, a chromophore, a specific binding protein, an antibody, a ligand-binding fragment of an antibody, an antigen, a receptor, a receptor ECD, a ligand-binding fragment of a receptor, a receptor ligand, biotin, and streptravidin..

123. A cellular microarray made by a method comprising:
engaging an arrayer having a plurality of pins with an embedding mold and a fluid temperature-sensitive matrix such that the matrix and the pins are contained within the embedding mold, wherein the embedding mold has a bottom surface;
freezing the matrix within the embedding mold to solidify the matrix;
removing the arrayer pins from the matrix and the embedding mold to form a plurality of wells disposed within the solid temperature-sensitive matrix;
inserting two or more biological samples into the plurality of wells to form an array of biological samples;
slicing the array to form one or more array slices, wherein each array slice has an array of transverse sections of biological sample corresponding to the array of biological samples;
mounting one or more of the array slices on a planar substrate surface; and
removing the temperature-sensitive matrix material from platform to form a microarray of transverse sections of biological sample.

124. The microarray of claim 123, wherein the planar substrate is a glass plate.

125. The microarray of claim 123, wherein the density of transverse biological sample sections in an array is at least 5 transverse sections/cm², at least 7 transverse sections/cm², at least 11 transverse sections/cm², at least 13 transverse sections/cm².

126. The microarray of claim 123, wherein one or more of the biological samples are tissue.

127. The microarray of claim 126, wherein the tissue is selected from the group consisting of normal tissue, diseased tissue, treated tissue, tissue from an adult organism, and tissue from an organism is a pre-adult stage of development.

128. The microarray of claim 123, wherein the engaging step further comprises coating the arrayer pins with a lubricating material

129. The microarray of claim 128, wherein the lubricating material is selected from a group consisting of glycerol, fatty acids, oil, grease, fat, or soap.

130. A cellular microarray made by a method comprising:
preparing a matrix having a plurality of wells disposed therein;
mixing a standard molecule with an embedding material to form an internal standard preparation, wherein the embedding material differs from the matrix in at least one physical or chemical property;
inserting the internal standard preparation into one or more of the plurality of wells in the matrix;
inserting a biological sample into one or more of the plurality of wells in the matrix;
slicing the array to form one or more array slices;
mounting one or more of the array slices on a planar substrate; and
removing the matrix from the substrate.

131. The method of claim 130, wherein the biological sample is not contained within a tube within the matrix.

132. The method of claim 130, wherein the standard molelcule is a polynucleotide selected from the group consisting of an RNA molecule and a DNA molecule.

133. The method of claim 130, wherein the standard molecule is a polypeptide.

134. The method of claim 133, wherein the polypeptide is selected from the group consisting of a receptor, a ligand-binding receptor fragment, a receptor ECD, a receptor ligand, an antibody, an antigen-binding antibody fragment, an antibody antigen, and an enzyme.

135. The method of claim 130, wherein the biological sample is selected from the group consisting of a cell suspension, a cell pellet, a cell lysate, a tissue, and a frozen tissue.

136. The method of claim 130, wherein the matrix is selected from the group consisting of a temperature-sensitive matrix, a mixture of resin-polyvinyl alcohol and polyethylene glycol, Optimal Cutting Temperature (OCT) matrix, paraffin, and gelatin.

137. The method of claim 130, wherein the embedding material comprises agarose.

138. A cellular microarray comprising:
a substrate comprising a planar surface;
one or more cellular biological samples on the surface, wherein the microarray lacks array matrix material.

139. The cellular microarray of claim 138, wherein the biological sample is selected from the group consisting of a cell suspension, a cell pellet, a cell lysate, a tissue, and a frozen tissue.

140. The cellular microarray of claim 138, wherein the array comprises transverse sections of the biological samples at a density of at least 5 samples/cm², at least 7 samples /cm², at least 11 samples/cm², and at least 13 samples/cm².

141. A cellular microarray comprising:
a substrate comprising a planar surface;
one or more cellular biological samples on the surface; and
one or more internal standard preparations on the surface, the internal standard preparation comprising a standard molecule admixed in an embedding material.

142. The cellular microarray of claim 141, wherein the biological sample is selected from the group consisting of a cell suspension, a cell pellet, a cell lysate, a tissue, and a frozen tissue.

143. The cellular microarray of claim 141, wherein the array comprises transverse sections of the biological samples at a density of at least 5 samples/cm², at least 7 samples/cm², at least 11 samples/cm², and at least 13 samples/cm².

144. The cellular microarray of claim 141, wherein the microarray lacks array matrix material.

145. The cellular microarray of claim 141 further comprising an orientation marker sample at at least one known location in relation to the one or more biological samples on the surface.

146. The cellular microarray of claim 141, wherein the orientation marker sample comprises a compound selected from the group consisting of a visible dye, a compound that non-specifically binds the standard molecule, cellulose, microgranular cellulose, and bentonite.

147. The method of claim 93, the method further comprising diagnosing colorectal cancer in a patient by determining at least 2-fold overexpression of p53 and at least 1.5-fold underexpression of hMLH1 in a biological sample from the patient.

148. The method of claim 93, the method further comprising diagnosing cancer in a patient by determining at least 2-fold overexpression, relative to normal control tissue, of VEGF in a biological sample, wherein the biological sample is a tissue selected from the group consisting of blood, muscle, nerve, brain, breast, prostate, heart, lung, liver, pancreas, spleen, thymus, esophagus, stomach, intestine, kidney, testis, ovary, uterus, hair follicle, skin, bone, bladder, and spinal cord.

149. A method of claim 93, the method further comprising diagnosing breast cancer in a patient by determining overexpression of Her2 gene or HER2 polypeptide in a breast tissue sample of the patient.

150. The method of claim 93, the method further comprising identifying a patient disposed to respond favorably to an ErbB antagonist for treating cancer, which method comprises detecting erbB gene amplification in tumor cells in a tissue sample from the patient.

151. The method of claim 150, wherein ErbB is HER2, the ErbB antagonist is an anti-HER2 antibody or HER2-binding fragment thereof, and erbB is Her2 gene.

152. The method of claim 151, wherein the anti-HER2 antibody is rhuMAb 4D5 (Herceptin®).

153. The method of claim 150, wherein detecting is by contacting a detectably labeled polynucleotide, comprising at least 20 contiguous nucleotides of the Her2 gene or its complementary sequence, with the sample.

154. The method of claim 93, the method further comprising diagnosing cancer in a patient by determining at least 1.5-fold overexpression of VEGF gene or VEGF polypeptide in a biological sample from the patient relative to expression in a control sample.

155. The method of claim 154, wherein the determining step comprises detecting the overexpression of VEGF gene by contacting the nucleic acid in the sample with a detectably labeled polynucleotide comprising at least 20 contiguous nucleotides of the VEGF gene or its complementary sequence.

156. The method of claim 155, wherein the determining step comprises detecting the overexpression of the VEGF polypeptide by contacting the VEGF polypeptide in the sample with a detectably labeled anti-VEGF antibody or binding fragment of the antibody.

157. The method of claim 93, the method further comprising diagnosing cancer in a patient by determining at least 1.5-fold overexpression of VEGF gene and HIF-1α in a biological sample from the patient relative to expression in control tissue samples.

158. The method of claim 157, wherein the determining step comprises detecting the overexpression of VEGF gene by contacting the nucleic acid in the sample with a detectably labeled polynucleotide comprising at least 20 contiguous nucleotides of the VEGF gene or its complementary sequence and detecting the overexpression of HIF-1α gene by contacting the nucleic acid in the sample with a detectably labeled polynucleotide comprising at least 20 contiguous nucleotides of the HIF-1α gene or its complementary sequence.

159. The method of claim 158, wherein the biological sample is a tissue selected from the group consisting of blood, muscle, nerve, brain, breast, prostate, heart, lung, liver, pancreas, spleen, thymus, esophagus, stomach, intestine, kidney, testis, ovary, uterus, hair follicle, skin, bone, bladder, and spinal cord.

160. The method of claim 159, wherein the biological sample is kidney tissue suspected of comprising renal cell carcinoma.
